(19) United States
(12) Patent Application Publication
(10) Pub. No.: US 2026/0090982 A1
(43) Pub. Date: Apr. 2, 2026

(54) TRANSDERMAL DELIVERY FORMULATIONS

(71) Applicant: LifeActive, Inc., Sheridan, WY (US)

(72) Inventors: Calvin Dexter MORRIS, Medford, OR (US); Burgess A. THOMASSON, JR., Fairhope, AL (US); Scott A. JENKINS, San Francisco, CA (US)

(73) Assignee: LifeActive, Inc., Sheridan, WY (US)

(21) Appl. No.: 19/411,130

(22) Filed: Dec. 5, 2025

Related U.S. Application Data
(63) Continuation-in-part of application No. PCT/US2024/032685, filed on Jun. 6, 2024.
(60) Provisional application No. 63/471,487, filed on Jun. 6, 2023.
(60) Provisional application No. 63/617,608, filed on Jan. 4, 2024.
(60) Provisional application No. 63/905,360, filed on Oct. 24, 2025.

(51) Int. Cl. A61K 9/00; A61K 9/107; A61K 31/047; A61K 31/05; A61K 31/07; A61K 31/121; A61K 31/155; A61K 31/192; A61K 31/198; A61K 31/205; A61K 31/355; A61K 31/375; A61K 31/401; A61K 31/4045; A61K 31/405; A61K 31/4172; A61K 31/4188; A61K 31/455; A61K 31/51; A61K 31/525; A61K 31/593; A61K 31/714; A61K 35/60; A61K 36/21; A61K 36/9066; A61K 38/06; A61K 45/06; A61K 47/12; A61K 47/24; A61K 47/44; A61P 25/28

(52) U.S. Cl. CPC A61K 9/0014; A61K 9/1075; A61K 31/047; A61K 31/05; A61K 31/07; A61K 31/121; A61K 31/155; A61K 31/192; A61K 31/198; A61K 31/205; A61K 31/355; A61K 31/375; A61K 31/401; A61K 31/4045; A61K 31/405; A61K 31/4172; A61K 31/4188; A61K 31/455; A61K 31/51; A61K 31/525; A61K 31/593; A61K 31/714; A61K 35/60; A61K 36/21; A61K 36/9066; A61K 38/063; A61K 45/06; A61K 47/12; A61K 47/24; A61K 47/44; A61P 25/28

(57) ABSTRACT

Provided are transdermal delivery formulations, and methods for the manufacture thereof, for the epicutaneous administration of therapeutic agents, drugs, and nutrients to human subjects; veterinary animals; domesticated or undomesticated animals, plants or insects; or agricultural animals, plants, or insects. Transdermal delivery formulations disclosed herein comprise a homogenous mixture of (a) a transdermal accelerant comprising a weak organic acid having a pKa greater than 2.0 and (b) a microemulsion comprising a nonionic emulsifier, water, and a cis-unsaturated long-chain fatty acid. Fatty acid microemulsions are combined with the acidified transdermal accelerants having a pH greater than 1.0 (typically from 1.5 to 2.5) to yield a homogenous transdermal delivery formulation comprising fatty acid micelles and/or liposomes that encapsulate one or more compound, such as a nutrient or a drug, and incorporate one or more cis-unsaturated fatty acid having a 12 to 26 carbon chain that includes one or more double bond in a cis configuration. Transdermal delivery formulations exhibit ideal solubility and absorption properties in high humidity conditions, such as in a warm to hot shower or sauna.

### CROSS REFERENCE TO RELATED APPLICATIONS

This patent application claims priority to U.S. Provisional Application Ser. No. 63/905,360, filed on Oct. 24, 2025, and is a continuation-in-part of PCT Application Serial No. PCT/US2024/032685, filed on Jun. 6, 2024. PCT Application Serial No. PCT/US2024/032685 claims priority to U.S. Provisional Application Ser. No. 63/617,608, filed on Jan. 4, 2024, and U.S. Provisional Application Ser. No. 63/471,487, filed on Jun. 6, 2023. Each of the above-mentioned patent applications is incorporated herein by reference in its entirety.

### TECHNICAL FIELD

The present disclosure relates generally to emulsion formulations for delivery of agent(s), and methods of preparing and administering the same.

### BACKGROUND

Systemic delivery of therapeutic agents, drugs, or nutrients are presented with challenges to stability and acceptability. Enteric delivery formulations are exposed to extreme environments, requiring formulations that resist strong acids. Injected formulations are difficult to self-administer and result in biohazardous waste products in the form of used needles. Such challenges affect compliance to therapeutic regimens and increase cost of treatment. Such challenges affect the outcome of treatment. Topical delivery provides for access to the vast absorption area of the skin, is simple to administer, and does not generate hazardous waste. Current topical formulations provide for delivery to the stratum corneum and do not penetrate the stratum corneum to deliver components to the circulatory system. There is a need for systemic delivery formulations that provide for delivery through the stratum corneum to the circulation.

### SUMMARY

The present disclosure fulfills unmet needs in the art for efficient and systemic epicutaneous administration of drugs, nutrients, and other compounds to a human subject; veterinary animal; domesticated or undomesticated animal, plant or insect; or agricultural animal, plant, or insect. The transdermal delivery formulations disclosed herein provide unexpected and surprising advantages over existing technologies for the transdermal delivery of therapeutic agents, drugs, and nutrients, to achieve the efficient and systemic epicutaneous administration of individual compounds and mixtures compounds, including therapeutic agents, drugs, and nutrients, to a human subjects; veterinary animals; domesticated or undomesticated animals, plants or insects; or agricultural animals, plants, or insects in need thereof.

Provided herein are transdermal delivery formulations for epicutaneous administration, comprising: (a) a transdermal accelerant comprising: about 600-2000 ml total of one or more weak organic acids comprising lactic acid, acetic acid, formic acid, citric acid, oxalic acid, gallic acid, malic acid, maleic acid, malonic acid, succinic acid, tartaric acid, fumaric acid, or any combination thereof; and a carrier comprising: about 400-1000 ml total of one or more emulsifiers; about 400-1000 ml distilled water; and about 400-1000 ml total of one or more oils comprising one or more unsaturated long-chain fatty acids comprising adrenic acid, arachidonic acid, arachidic acid, behenic acid, brassidic acid, cervonic acid, cis-vaccenic acid, dihomo-γ-linolenic acid, docosadienoic acid, eicosadienoic acid, eicosapentaenoic acid, eicosatetraenoic acid, eicosenoic acid, elaidic acid, erucic acid, gadoleic acid, gondoic acid, herring acid, lauric acid, lignoceric acid, linoleic acid, linolelaidic acid, margaric acid, margoleic acid, mead acid, myristoleic acid, nervonic acid, oleic acid, ozubondo acid, palmitic acid, palmitoleic acid, trans-palmitoleic acid, paullinic acid, petroselinic acid, pinolenic acid, sapienic acid, sardine acid, stearic acid, stearidonic acid, tetracosapentaenoic acid, α-linolenic acid, γ-linolenic acid, or any combination thereof, wherein the transdermal accelerant and the carrier are combined to form a transdermal delivery formulation, wherein epicutaneous application of the transdermal formulation and one or more active agents provides for delivery of the one or more active agents through the stratum corneum.

Provided herein are transdermal delivery formulations for epicutaneous administration, comprising: (a) a transdermal accelerant comprising at least one weak organic acid having a pKa from about 2.0 to about 6.0; and (b) a carrier comprising one or more emulsifiers, water, and one or more oils comprising an unsaturated long-chain fatty acid, wherein the transdermal delivery formulation comprises: at least about 20% by weight of the transdermal accelerant; and at least about 50% by weight of the carrier, wherein epicutaneous application of the transdermal delivery formulation and one or more active agents provides for delivery of the one or more active agents through the stratum corneum. Further provided herein are transdermal delivery formulations, wherein the transdermal accelerant and the carrier are present at a ratio of about 8:1, 7:1, 6:1, 5:1, 4:1, 3:1, 2:1, 1:1, 1:2, 1:3, 1:4, 1:5, 1:6, 1:7, or 1:8 by weight. Further provided herein are transdermal delivery formulations, wherein the transdermal delivery formulation comprises about 20%, about 25%, about 30%, about 35%, about 40%, about 45%, about 50%, about 55%, about 60%, about 65%, about 70%, about 75%, or about 80% (w/w) of the transdermal accelerant. Further provided herein are transdermal delivery formulations, wherein the transdermal delivery formulation comprises about 60%, about 65%, about 70%, about 75%, or about 80% w/w of the carrier. Further provided herein are transdermal delivery formulations, wherein a transdermal delivery formulation of a weight of about 3100 g comprises: a transdermal accelerant of a weight of from about 200 g to about 2480 g; and a carrier of a weight from about 620 g to about 2900 g. Further provided herein are transdermal delivery formulations, wherein the transdermal accelerant comprises a weight of about 1050 g and the carrier comprises a weight of about 2050 g. Further provided herein are transdermal delivery formulations, wherein the transdermal accelerant comprises 1, 2, 3, 4, 5, 6, 7, 8, 9 or 10 weak organic acids. Further provided herein are transdermal delivery formulations, wherein the transdermal accelerant comprises 2 weak organic acids. Further provided herein are transdermal delivery formulations, wherein the transdermal accelerant comprises 2 weak organic acids at a ratio by weight of about 1:0, 1:1, 1:2, 1:3, 1:4, 1:5, 1:6, 1:7, 1:8, 1:9, 1:10, 1:100 or 1:1000 relative to each other. Further provided herein are transdermal delivery formulations, wherein the transdermal accelerant comprises about 80%, about 85%, about 90%, about 95%, about 97%, about 99% (w/w) of one or more weak organic acids. Further provided herein are transdermal delivery formulations, wherein the at least one weak organic acid has a median pKa of from 3.0 to 5.5. Further provided herein are transdermal delivery formulations, wherein the at least one weak organic acid has a median pKa of from 4.0 to 5.0. Further provided herein are transdermal delivery formulations, wherein the at least one weak organic acid has a median pKa of about 4.6. Further provided herein are transdermal delivery formulations, wherein the at least one weak organic acid comprises a mono, di or tri carbonic acid of chain length (R) between 1-16. Further provided herein are transdermal delivery formulations, wherein the at least one weak organic acid comprises a mono or poly hydroxy moiety of 0-14. Further provided herein are transdermal delivery formulations, wherein the at least one weak organic acid comprises a linear, branched, or cyclic structure. Further provided herein are transdermal delivery formulations, wherein the structure is saturated or unsaturated. Further provided herein are transdermal delivery formulations, wherein the at least one weak organic acid comprises lactic acid, acetic acid, formic acid, citric acid, oxalic acid, gallic acid, malic acid, maleic acid, malonic acid, succinic acid, tartaric acid, fumaric acid, or any combination thereof. Further provided herein are transdermal delivery formulations, wherein said weak organic acid is citric acid or acetic acid. Further provided herein are transdermal delivery formulations, wherein said transdermal accelerant comprises citric acid and acetic acid. Further provided herein are transdermal delivery formulations, wherein the acetic acid is apple cider vinegar. Further provided herein are transdermal delivery formulations, wherein the transdermal accelerant comprises from about 100 g to about 700 g citric acid and from about 300 g to about 1300 g apple cider vinegar. Further provided herein are transdermal delivery formulations, wherein the transdermal accelerant comprises about 100 g, about 150 g, about 200 g, about 250 g, about 300 g, about 350 g, about 400 g, about 450 g, about 500 g, about 550 g, about 600 g, about 650 g, about 700 g citric acid. Further provided herein are transdermal delivery formulations, wherein the transdermal accelerant comprises about 300 g, about 350 g, about 400 g, about 450 g, about 500 g, about 550 g, about 600 g, about 650 g, about 700 g, about 750 g, about 800 g, about 850 g, about 900 g, about 950 g, about 1000 g, about 1100 g, about 1200 g, about 1300 g apple cider vinegar. Further provided herein are transdermal delivery formulations, wherein the carrier comprises at least one emulsifier, at least one oil comprising a long-chain fatty acid, and water at a ratio by weight of about 1:1:1, 1:1:2, 1:1:3, 1:1:4, 1:1:5, 1:1:6, 1:2:2, 1:2:3, 1:2:4, 1:2:5, 1:2:6, 1:3:3, 1:3:4, 1:3:5, 1:3:6, 1:4:4, 1:4:5, 1:4:6, 1:5:5, 1:5:6, 1:6:6, 2:2:3, 2:2:4, 2:2:5, 2:2:6, 2:3:3, 2:3:4, 2:3:5, 2:3:6, 2:4:4, 2:4:5, 2:4:6, 2:5:5, 2:5:6, 2:6:6, 3:3:4, 3:3:5, 3:3:6, 3:4:4, 3:4:5, 3:4:6, 3:5:5, 3:5:6, 3:6:6, 4:4:5, 4:4:6, 4:5:5, 4:5:6, 4:6:6, 5:5:6, or 5:6:6. Further provided herein are transdermal delivery formulations, wherein the carrier comprises about 15%, about 20%, about 25%, about 30%, about 35%, about 40%, about 45%, about 50%, about 55%, or about 60% w/w of one or more emulsifiers. Further provided herein are transdermal delivery formulations, wherein the carrier comprises from about 300 to about 1500 g, from about 500 g to about 1000 g, from about 700 g to about 800 g one or more emulsifiers. Further provided herein are transdermal delivery formulations, wherein the emulsifier comprises a non-ionic emulsifier. Further provided herein are transdermal delivery formulations, wherein said nonionic emulsifier is selected from the group consisting of lecithin, carboxylmethylcellulose, a sorbitan ester, and a polysorbate. Further provided herein are transdermal delivery formulations, wherein said nonionic emulsifier is a sorbitan ester selected from the group consisting of sorbitan monolaurate, sorbitan monostearate, sorbitan tristearate, and sorbitan monooleate. Further provided herein are transdermal delivery formulations, wherein said nonionic emulsifier is a polysorbate selected from a class of emulsifiers including but not limited to of polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and polyoxyethylene (20) sorbitan monooleate (polysorbate 80). Further provided herein are transdermal delivery formulations, wherein said polysorbate comprises polysorbate 20, polysorbate 40, polysorbate 60, or polysorbate 80. In some embodiments, the polysorbate is polysorbate 80. Further provided herein are transdermal delivery formulations, wherein the carrier comprises about 15%, about 20%, about 25%, about 30%, about 35%, about 40%, about 45%, about 50%, about 55%, about 60%, about 65%, about 70% w/w water. Further provided herein are transdermal delivery formulations, wherein the carrier comprises from about 300 to about 1500 g, about 500 g to about 1000 g, from about 700 to about 800 g water. Further provided herein are transdermal delivery formulations, wherein said water is distilled water or deionized water. In some embodiments, the carrier herein comprises the at least one emulsifier, and the at least one oil comprising a long-chain fatty acid. In illustrative embodiments, the carrier herein does not comprise water, distilled water, or deionized water. The carrier in certain embodiments can have less than 10%, 9%, 8%, 7%, 6%, 5%, 4%, 3%, 2%, 1%, or 0.5% water, and in illustrative embodiments has no water. The carrier in certain embodiments can have less than 10%, 9%, 8%, 7%, 6%, 5%, 4%, 3%, 2%, 1%, or 0.5% added water (e.g., purified, distilled, or tap) and in illustrative embodiments has no added water (e.g., purified, distilled, or tap). Further provided herein are transdermal delivery formulations, wherein the carrier comprises about 5% (w/w), about 10%, about 15%, about 20%, about 25%, about 30%, about 35%, about 40%, about 45%, about 50%, about 55%, about 60%, about 65%, about 70% of at least one oil comprising an unsaturated long-chain fatty acid. Further provided herein are transdermal delivery formulations, wherein the carrier comprises two oils comprising an unsaturated long-chain fatty acid, wherein the two oils are in a ratio by weight of about 1:2, 1:3, 1:4, 1:5, 1:6, 1:7, 1:8, 1:9, 1:10, 1:100, 1:1000 relative to each other. Further provided herein are transdermal delivery formulations, wherein the carrier comprises about 300 g, about 400 g, about 500 g, about 600 g, about 700 g, about 800 g, about 900 g, about 1000 g, about 1100 g, about 1200 g, about 1300 g, about 1400 g, about 1500 g one or more oils comprising an unsaturated long-chain fatty acid. Further provided herein are transdermal delivery formulations, wherein said unsaturated long-chain fatty acid comprises a chain of from 12 to 26 carbons. Further provided herein are transdermal delivery formulations, wherein said at least one unsaturated long-chain fatty acid comprises one or more double bonds in a cis configuration. Further provided herein are transdermal delivery formulations, wherein said at least one unsaturated long-chain fatty acid comprises adrenic acid, arachidonic acid, arachidic acid, behenic acid, brassidic acid, cervonic acid, cis-vaccenic acid, dihomo-γ-linolenic acid, docosadienoic acid, eicosadienoic acid, eicosapentaenoic acid, eicosatetraenoic acid, eicosenoic acid, elaidic acid, erucic acid, gadoleic acid, gondoic acid, herring acid, lauric acid, lignoceric acid, linoleic acid, linolelaidic acid, margaric acid, margoleic acid, mead acid, myristoleic acid, nervonic acid, oleic acid, ozubondo acid, palmitic acid, palmitoleic acid, trans-palmitoleic acid, paullinic acid, petroselinic acid, pinolenic acid, sapienic acid, sardine acid, stearic acid, stearidonic acid, tetracosapentaenoic acid, α-linolenic acid, γ-linolenic acid, or any combination thereof. Further provided herein are transdermal delivery formulations, wherein said at least one unsaturated long-chain fatty acid is oleic acid or linoleic acid. Further provided herein are transdermal delivery formulations, wherein said at least one long-chain fatty acid comprises oleic acid and linoleic acid. Further provided herein are transdermal delivery formulations, wherein the one or more oils comprising an unsaturated long-chain fatty acid is one or more plant oils. Further provided herein are transdermal delivery formulations, wherein said one or more plant oils is selected from the group consisting of vegetable oil, nut oil and seed oil. Further provided herein are transdermal delivery formulations, wherein said one or more plant or animal oils comprises macadamia oil, maracuja (passion fruit) oil, safflower oil, sunflower oil, olive oil, avocado oil, canola oil, coconut oil, corn oil, cottonseed oil, flaxseed/linseed oil, grape seed oil, hemp seed oil, palm oil, peanut oil, rice bran oil, sesame oil, soybean oil, Brazil nut oil, almond oil, walnut oil, pecan oil, jojoba oil, chia seed oil, wallflower seed, mustard oil, borage oil, black currant oil, evening primrose oil, chicken fat, cartilage oil, cod liver oil, herring oil, mackerel oil, salmon oil, menhaden oil, sardine oil, or any combination thereof. Further provided herein are transdermal delivery formulations, wherein said one or more plant oils is macadamia oil or maracuja (passion fruit) oil, or a combination thereof. Further provided herein are transdermal delivery formulations, wherein said one or more plant oils comprises macadamia oil and maracuja (passion fruit) oil. Further provided herein are transdermal delivery formulations, wherein the carrier comprises from about 100 to about 600 g, from about 200 g to about 500 g, from about 300 g to about 400 g macadamia oil and from about 100 to about 600 g, from about 200 g to about 500 g, from about 300 g to about 400 g maracuja oil. Further provided herein are transdermal delivery formulations, wherein the transdermal delivery formulation comprises a lotion, a gel, a cream, an ointment, a liniment, a paste, a film, an encapsulation, or a liquid. Further provided herein are transdermal delivery formulations, wherein said transdermal accelerant further comprises a nitrate source. Further provided herein are transdermal delivery formulations, wherein said nitrate source is a plant-based nitrate source. Further provided herein are transdermal delivery formulations, wherein said plant-based nitrate source comprises from the group consisting of arugula, spinach, leafy green vegetables, beetroot, or any combination thereof. Further provided herein are transdermal delivery formulations, wherein said plant-based nitrate source is beetroot, and wherein the beetroot is a dried beetroot powder. Further provided herein are transdermal delivery formulations, wherein the transdermal accelerant comprises about 2.0%, about 2.5%, about 3.0%, about 3.5%, about 4.0%, about 4.5%, about 5.0%, about 5.5%, about 6.0%, about 6.5%, about 7.0%, about 7.5%, or about 8.0% w/w dried beetroot powder. Further provided herein are transdermal delivery formulations, wherein the transdermal accelerant comprises about 10 g, about 15 g, about 20 g, about 25 g, about 30 g, about 35 g, about 40 g, about 45 g, about 50 g, about 55 g, about 60 g, about 65 g, about 70 g, about 75 g, about 80 g, about 85 g, about 90 g, about 95 g, or about 100 g dried beetroot powder. Further provided herein are transdermal delivery formulations, further comprising a viscosity enhancer, a nutrient, a plant powder or extract, an amino acid, a vitamin, or any combination thereof. Further provided herein are transdermal delivery formulations, wherein said formulation comprises a viscosity enhancer selected from the group consisting of lecithin, aloe vera, glycerin, a plant oil, an animal oil, and collagen. Further provided herein are transdermal delivery formulations, wherein said formulation comprises a nutrient selected from the group consisting of acetyl-L-carnitine, alpha lipoic acid, arginine, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide. Further provided herein are transdermal delivery formulations, wherein said formulation comprises a plant powder or extract selected from the group consisting of bacopa powder, bamboo extract powder, beet powder, blueberry extract, Ginkgo biloba, ginger, grape seed extract, green tea, jojoba, nutmeg, olive leaf, pomegranate, and turmeric. Further provided herein are transdermal delivery formulations, wherein said formulation comprises an amino acid selected from the group consisting of alanine, arginine, leucine, isoleucine, valine, glutamine, glycine, histidine, leucine, lysine, methionine, proline, serine, threonine, and valine. Further provided herein are transdermal delivery formulations, wherein said formulation comprises a vitamin selected from the group consisting of vitamin A, vitamin B-1, vitamin B-2, vitamin B-3, vitamin B-7 & 8 inositol, vitamin B-9 (folic acid), vitamin B-12, vitamin C, vitamin D-3, and vitamin E. Further provided herein are transdermal delivery formulations, wherein said transdermal formulation has a pH of from 2.0 to 6.0. Further provided herein are transdermal delivery formulations, wherein said transdermal formulation has a pH of from 3.0 to 5.0. Further provided herein are transdermal delivery formulations, wherein said transdermal formulation has a pH of from 3.5 to 4.5. Further provided herein are transdermal delivery formulations, wherein said transdermal formulation has a viscosity at 20° C. of from 500 to 10,000 centipoise (cP) or from 1,000 to 5,000 cP, or from 1,500 to 4,000 cP, or from 2,000 to 3,000 cP. Further provided herein are transdermal delivery formulations, wherein said transdermal formulation has a viscosity of about 2,500 cP. Further provided herein are transdermal delivery formulations, wherein the transdermal delivery formulation forms an emulsion comprising a population of droplets from about 10 nm to about 300 nm in diameter.

Provided herein are transdermal delivery formulations as provided herein, further comprising one or more components for enhancing nutrition and optimizing health, immune support, restful sleep, exercise performance and nitric oxide metabolism, cognitive function, or any combination thereof. Further provided herein are transdermal delivery formulations, further comprising one or more of a hydration electrolyte, cinnamic acid, phenylalanine, resveratrol, and carnitine. Further provided herein are transdermal delivery formulations, further comprising one or more of gamma-aminobutyric acid (GABA), melatonin, and tryptophan. Further provided herein are transdermal delivery formulations, further comprising one or more of vitamin D, vitamin C, citrulline, ribose ATP blood carrier, beetroot, curcumin (turmeric), fish oil, and threonine. Further provided herein are transdermal delivery formulations, further comprising one or more of silicon, leucine, lysine, isoleucine, valine, threonine, phenylalanine, methionine, histidine, tryptophan, vitamin B-5 (pantothenic acid), vitamin B-7 (biotin), vitamin K, acetyl L carnitine, palmitoyl-ethanolamide (PEA), taurine, periwinkle, artichoke, bacopa, Ginkgo biloba, nutmeg, alanine, and tyrosine. Further provided herein are transdermal delivery formulations, wherein the formulation comprises: about 12.0% (w/w) citric acid powder; about 20.5% (w/w) apple cider vinegar; about 1.4% (w/w) beetroot powder; about 23.8% (w/w) polysorbate 80; about 22.5% (w/w) distilled water; about 9.9% (w/w) macadamia oil; and about 9.8% (w/w) maracuja oil. Further provided herein are transdermal delivery formulations, wherein the formulation comprises: about 374 g citric acid powder; about 640 g apple cider vinegar; about 44.4 g beetroot powder; about 742 g polysorbate 80; about 700 g distilled water; about 308 g macadamia oil; and about 308 g maracuja oil. Further provided herein are transdermal delivery formulations, further comprising one or more additional ingredients, wherein the additional ingredients comprise: acetyl-l-carnitine (alc), alanine, alpha lipoic acid, arginine, artichoke extract, ashwagandha, bacopa powder, bamboo extract powder, basil powder, beet powder, calcium carbonate, blueberry extract, aloe vera, choline bitartrate, collagen protein peptides, collagen powder, citrulline malate, curcumin powder, creatine, CoQ10, kale powder, folic acid, gingko biloba, glutamine, glycerin, ginger, glycine, grape seed extract, green tea, inositol (vitamins B7/B8), histidine, jojoba, pomegranate powder, magnesium glycinate, leucine, lysine, lecithin (sun flower), niacinamide (vitamin B3), nutmeg powder, niacin, methionine, NALT (acetyl tyrosine), NAC, lutein, olive leaf, pea powder, periwinkle, phenylalanine, potassium, proline, ribose, DAA (d-aspartic acid), serine, hyaluronic acid, taurine, threonine, tryptophan, turmeric, theanine, valine (amino acid), valerian root powder, zinc oxide, vitamin A, vitamin B complex, vitamin B-1, vitamin B-2, vitamin B-5, vitamin B-6 (pyridoxine), vitamin B-7&8, vitamin B-9 (folic acid), vitamin B-12, vitamin C (ascorbic acid), vitamin D-3 (cholecalciferol), vitamin E, vitamin K-2, ginseng, isoleucine, almond oil, broccoli seed oil, collagen liquid, avocado oil, chamomile liquid, vitamin E oil, glycerin (conditioning), grapefruit seed, grape seed oil, gotu kola oil, kava liquid, virgin algae oil (fish oil), jojoba extract oil (organic), lavender oil, macadamia nut oil, meadowfoam seed oil, peppermint oil, maracuja oil (passionfruit), primrose oil, pomegranate oil, BCAA, GABA, collagen oil, virgin algae oil, ashwagandha powder, collagen peptides, guarana powder, and huperzine. Further provided herein are transdermal delivery formulations, wherein the transdermal delivery formulation comprises more than one, more than 2, more than 3, more than 4, more than 5, more than 6, more than 7, more than 8, more than 9, more than 10, more than 11, more than 12, more than 13, more than 14, more than 15, more than 16, more than 17, more than 18, more than 19, more than 20 of the additional ingredients. Further provided herein are transdermal delivery formulations, wherein the one or more active agents comprises a therapeutic agent, a nutraceutical, or a combination thereof. Further provided herein are transdermal delivery formulations, wherein the therapeutic agent comprises a biologic, a protein, a peptide, a small molecule, a macromolecule, a nucleic acid, another pharmaceutically or physiologically active ingredient, or any combination thereof.

Provided herein are methods of transdermal delivery, comprising epicutaneous application of the transdermal delivery formulation as described herein. Further provided herein are methods of transdermal delivery, further comprising overlaying an ionized water on to the epicutaneously-applied transdermal delivery formulation.

Provided herein are methods of manufacture comprising: generating a transdermal accelerant comprising: about 600-2000 ml total of one or more weak organic acids having a pKa from about 2.0 to about 6.0; and generating a carrier comprising: about 400-1000 ml non-ionic surfactant; about 400-1000 ml distilled water; and about 400-1000 ml total of one or more oils comprising one or more of adrenic acid, arachidonic acid, arachidic acid, behenic acid, brassidic acid, cervonic acid, cis-vaccenic acid, dihomo-y-linolenic acid, docosadienoic acid, eicosadienoic acid, eicosapentaenoic acid, eicosatetraenoic acid, eicosenoic acid, elaidic acid, erucic acid, gadoleic acid, gondoic acid, herring acid, lauric acid, lignoceric acid, linoleic acid, linolelaidic acid, margaric acid, margoleic acid, mead acid, myristoleic acid, nervonic acid, oleic acid, ozubondo acid, palmitic acid, palmitoleic acid, trans-palmitoleic acid, paullinic acid, petroselinic acid, pinolenic acid, sapienic acid, sardine acid, stearic acid, stearidonic acid, tetracosapentaenoic acid, α-linolenic acid, γ-linolenic acid, or any combination thereof, combining the transdermal accelerant and the carrier to generate a transdermal delivery formulation; applying an energy to the transdermal delivery formulation to generate an emulsion, wherein the emulsion comprises a population of emulsion droplets from about 10 to about 300 nm in diameter. Further provided herein are methods of manufacture, wherein the one or more weak organic acids comprise mono, di or tri carbonic acids of chain lengths (R) between 1-16. Further provided herein are methods of manufacture, wherein the one or more weak organic acids comprise mono or poly hydroxy moieties of 0-14. Further provided herein are methods of manufacture, wherein the one or more weak organic acids comprise a linear, branched, or cyclic structure. Further provided herein are methods of manufacture, wherein the structure is saturated or unsaturated. Further provided herein are methods of manufacture, wherein the one or more weak organic acids comprises lactic acid, acetic acid, formic acid, citric acid, oxalic acid, gallic acid, malic acid, maleic acid, malonic acid, succinic acid, tartaric acid, fumaric acid, or any combination thereof.

Further provided herein are methods of manufacture, further comprising adding one or more active agents to the transdermal delivery formulation. Further provided herein are methods of manufacture, wherein the one or more active agents comprises a drug, therapeutic agent, a nutraceutical, or a combination thereof. Further provided herein are methods of manufacture, wherein the therapeutic agent comprises a biologic, a protein, a peptide, a small molecule, a macromolecule, a nucleic acid, another pharmaceutically or physiologically active ingredient, or any combination thereof. Further provided herein are methods of manufacture, wherein the energy is in the form of a shear force, a cavitation, an impact, or any combination thereof. Further provided herein are methods of manufacture, wherein the energy is applied by vortexing, shearing, stirring, shaking, vortexing, shearing, sonicating, homogenizing, blending, tumbling, extruding, milling, grinding, lyophilization, electrolyzing, heating, cooling, atomizing, or any combination thereof.

Provided herein are methods of treatment of a condition comprising epicutaneous application of a transdermal delivery formulation described herein and one or more active agents. Further provided herein are methods of treatment, wherein the one or more active agents comprises a drug, a therapeutic agent, a nutraceutical, or a combination thereof. Further provided herein are methods of treatment, wherein the therapeutic agent comprises a biologic, a protein, a peptide, a small molecule, a macromolecule, a nucleic acid, another pharmaceutically or physiologically active ingredient, or any combination thereof.

Provided herein are methods of enhancing a feature or condition comprising epicutaneous application of a transdermal delivery formulation described herein and one or more active agents. Further provided herein are methods of enhancing a feature or condition, wherein the feature is a cosmetic feature. Further provided herein are methods of enhancing a feature or condition, wherein the condition is nutrition, general health, quality of sleep, exercise performance, nitric oxide, physiology, metabolism, cognitive function, or any combination thereof.

Provided herein are methods of systemic delivery of an active agent, comprising epicutaneous application of a transdermal delivery formulation described herein. Further provided herein are methods of systemic delivery, further comprising overlaying an ionized water on to the epicutaneously-applied transdermal delivery formulation. Further provided herein are methods of systemic delivery, wherein the one or more active agents comprises a therapeutic agent, a nutraceutical, or a combination thereof. Further provided herein are methods of systemic delivery, wherein the therapeutic agent comprises a biologic, a protein, a peptide, a small molecule, a macromolecule, a nucleic acid, another pharmaceutically or physiologically active ingredient, or any combination thereof.

These and other related aspects of the present disclosure will be better understood in view of the following drawings and detailed description, which exemplify certain aspects of the various embodiments.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a drawing that depicts the major elements of human skin, including, in sequence from outer layer to inner layer, the epidermis, the dermis, and the hypodermis. As shown, the epidermis comprises an outer surface layer of stratum corneum, which covers the stratum lucidum, the stratum granulosum, stratum spinosum, and basal layer at the inner surface of the epidermis. Vascularization originates at the interface between the dermis and hypodermis, and capillaries extend into the dermis.

FIG. 2 is a drawing that depicts liposomes and micelles comprised within cis-unsaturated fatty acid microemulsions and transdermal delivery formulations disclosed herein.

FIG. 3 is a drawing that depicts a Franz diffusion cell for use in in vitro models for testing transdermal delivery formulations as disclosed in Example 3 by, for example, utilizing non-viable skin to measure penetration and permeation only or utilizing fresh, metabolically active skin to simultaneously measure permeation and skin metabolism.

FIGS. 4A-4F provides bar graphs depicting the data presented in Example 5, Table 9. FIG. 4A is a bar chart showing subject scores at baseline and after tests 1, 2, and 3 in the Trails A test for Attention. FIG. 4B is a bar chart showing subject scores at baseline and after tests 1, 2, and 3 in the Trails B test for Mental Flexibility. FIG. 4C is a bar chart showing subject scores at baseline and after tests 1, 2, and 3 in the Digital Symbol Substitution test for Executive Function. FIG. 4D is a bar chart showing subject scores at baseline and after tests 1, 2, and 3 in the Stroop test for Executive Function. FIG. 4E is a bar chart showing subject scores at baseline and after tests 1, 2, and 3 in the Immediate Recognition test for Memory. FIG. 4F is a bar chart showing subject scores at baseline and after tests 1, 2, and 3 in the Delayed Recognition test for Memory.

FIGS. 5A-5N illustrate relative amounts of sub-2 micron diameter particles in emulsion formulations as measured by percent intensity: FIG. 5A and FIG. 5B show size distribution of Batch 1, sample A, diluted to 1:200 and 1:400, respectively. FIG. 5C and FIG. 5D show size distribution of Batch 1, sample B, diluted to 1:200 and 1:400, respectively. FIG. 5E and FIG. 5F show size distribution of Batch 2, sample A, diluted to 1:200 and 1:400, respectively. FIG. 5G and FIG. 5H show size distribution of Batch 2, sample B, diluted to 1:200 and 1:400, respectively. FIG. 5I and FIG. 5J show size distribution of Batch 3, sample A, diluted to 1:200 and 1:400, respectively. FIG. 5K and FIG. 5L show size distribution of Batch 3, sample B, diluted to 1:200 and 1:400, respectively. FIG. 5M and FIG. 5N show size distribution of Batch 1, sample A, diluted in tap or HPLC water to 1:200 and 1:400, respectively.

FIG. 6 is a line graph showing blood pressure, heart rate, blood oxygen, and nitric oxide levels measured at time points to 360 minutes after topical application of an emulsion comprising nitric oxide precursors and caffeine.

FIG. 7 is a table showing nitric oxide test strips used at timepoints after application of an emulsion as described herein, indicating increased nitric oxide levels 12-14 hours after topical application of an emulsion comprising nitric oxide precursors.

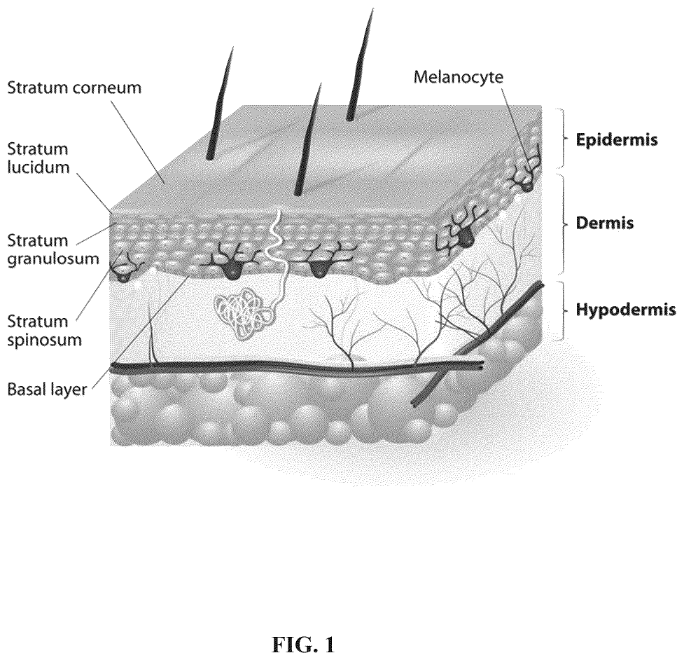

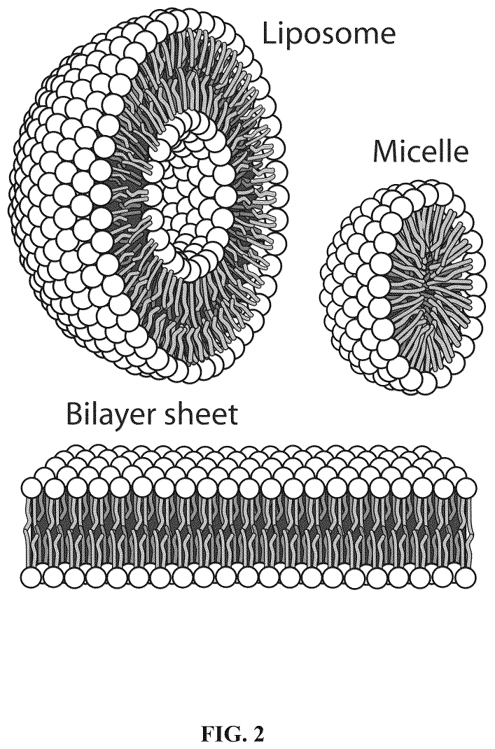

### DETAILED DESCRIPTION

Provided herein are transdermal delivery formulations that exhibit unexpected and surprising advantages over technologies that are currently available in the art for the efficient systemic epicutaneous administration of therapeutic agents, drugs, and nutrients, to the bloodstream of a human subject; veterinary animal; domesticated or undomesticated animal, plant or insect; or agricultural animal, plant, or insect. Transdermal delivery formulations disclosed herein comprise a homogenous mixture of (a) a transdermal accelerant comprising a weak organic acid having a pKa greater than 2.0 and (b) a microemulsion comprising a nonionic emulsifier, distilled water, and a cis-unsaturated long-chain fatty acid.

Transdermal delivery formulations disclosed herein comprise acidified micelles and/or liposomes that encapsulate a compound or cluster of compounds, such as a therapeutic agent, drug, or nutrient. These transdermal delivery formulations are made by preparing separately (a) a transdermal accelerant comprising a weak organic acid solution having a pKa greater than 2.0 and (b) a microemulsion comprising a nonionic emulsifier, water, and a cis-unsaturated long-chain fatty acid or acids. Those fatty acids microemulsions are combined with the acidified transdermal accelerants having a pH greater than 1.0 (typically from 1.5 to 2.5) to yield a homogenous transdermal delivery formulation comprising fatty acid micelles and/or liposomes that encapsulate one or more compounds, such as a therapeutic agent, a drug or nutrient. Those micelles and/or liposomes incorporate one or more cis-unsaturated fatty acid having a 12 to 26 carbon chain that includes one or more double bond in a cis configuration. The resulting micelles and/or liposomes yield transdermal delivery formulations that exhibit ideal solubility and absorption properties.

While oral delivery is the most common route for the enteral administration of therapeutic agents, drugs, and nutrients, it is unsuitable for delivery of therapeutic agents, drugs, and nutrients that are unstable in the acidic environment of the stomach, which can destroy their biological activity or otherwise block bioavailability. Many therapeutic agents, drugs, and nutrients are poorly absorbed in the intestines and/or are subject to first-pass metabolism wherein the concentration of an active drug or nutrient enters the hepatic portal system and is absorbed and/or metabolized in the liver before reaching the site of action or systemic circulation. Therapeutic agents, drugs, or nutrients administered sublingually (i.e. placed under the tongue) diffuse into the capillary network, are rapidly absorbed, and enter systemic circulation directly, thereby avoiding the gastrointestinal tract and are less susceptible to first-pass metabolism. Moreover, oral delivery is impractical for delivery to unconscious patients or when acute onset is required.

This disclosure will be better understood in view of the following definitions, which are provided for clarification and are not intended to limit the scope of the subject matter that is disclosed herein.

### Definitions

As used herein, the terms “transdermal delivery/administration” and “epicutaneous delivery/administration” refer interchangeably to the non-enteral, parenteral delivery/administration of drugs, nutrients, and other compounds to a subject through the skin to, thereby, avoid the adverse, degradative environment of the stomach and inefficient delivery of biologically active molecules through the small and large intestines in favor of the direct, non-invasive, and efficient delivery of molecules through the skin.

As used herein, the terms “transdermal delivery” and “transdermal penetration” refer synonymously to the passive diffusion of drugs, nutrients, and other compounds from the outer surface of the skin, through the stratum corneum and epidermis, and into the blood vasculature or via a shunt pathway, such as through hair follicles and associated sebaceous glands and the sweat ducts.

As used herein, the term “skin” refers primarily to “human skin,” which comprises three distinct but mutually dependent tissues, namely: 1. The stratified, a vascular, cellular epidermis; 2. Underlying dermis of connective tissues; and; 3. Hypodermis.

As used herein, the term “stratum corneum” refers to the outermost layer of skin. The stratum corneum is approximately 10 mm thick when dry but swells to several times this thickness when fully hydrated. It contains 10 to 25 layers parallel to the skin surface, which include dying or dead, keratinized cells, called corneocytes. Stratum corneum is flexible but relatively impermeable. The stratum corneum is the principal barrier for penetration. The barrier nature of the stratum corneum depends critically on its constituents: 75 to 80% proteins, 5 to 15% lipids, and 5 to 10% ondansetron material on a dry weight basis. Protein fractions predominantly contain alpha-keratin (70%) with some beta-keratin (10%) and cell envelope (5%). Lipid constituents vary with body site (neutral lipids, sphingolipids, polar lipids, cholesterol). Phospholipids are largely absent, a unique feature of mammalian membrane.

As used herein, the term “epidermis” refers to the multilayered envelope of the epidermis varies in thickness, depending on cell size and number of cell layers, ranging from 0.8 mm on palms and soles down to 0.06 mm on the eyelids. Stratum corneum and the remainder of the epidermis, also called viable epidermis, cover a major area of skin.

As used herein, the term “viable epidermis” refers to the cell layer that is situated beneath the stratum corneum, which varies in thickness from 0.06 mm on the eyelids to 0.8 mm on the palms. Going inwards, it includes various layers as stratum lucidum, stratum granulosum, stratum spinosum, and the stratum basale. In the basale layer, mitosis of the cells constantly renews the epidermis and this proliferation compensates the loss of dead horny cells from the skin surface. As the cells produced by the basale layer move outward, they alter morphologically and histochemically, undergoing keratinization to form the outermost layer of stratum corneum.

As used herein, the term “dermis” refers to the 3 to 5 mm thick layer and is composed of a matrix of connective tissue which contains blood vessels, lymph vessels, and nerves. The continuous blood supply has essential function in regulation of body temperature. It also provides nutrients and oxygen to the skin while removing toxins and waste products. Capillaries reach to within 0.2 mm of skin surface and provide sink conditions for most molecules penetrating the skin barrier. The blood supply thus keeps the dermal concentration of permeate very low, and the resulting concentration difference across the epidermis provides the essential driving force for transdermal permeation.

As used herein, the term “hypodermis” refers to the subcutaneous fat tissue that supports the dermis and epidermis. The hypodermis serves as a fat storage area, which helps to regulate temperature and provides nutritional support and mechanical protection. “Hypodermis” carries principal blood vessels and nerves to skin and may contain sensory pressure organs. For transdermal drug delivery, the drug has to penetrate through all these three layers and reach into systemic circulation while in case of topical drug delivery, only penetration through stratum corneum is essential and then retention of drug in skin layers is desired.

As used herein, the terms “transcorneal delivery” and “transcorneal penetration” refer synonymously to both the “intracellular” and “intercellular” penetration of a compound past the stratum corneum.

“Intracellular transcorneal delivery” and “intracellular transcorneal penetration” refer to the passing of a compound, typically a “hydrophilic compound” through the cells of the stratum corneum. As stratum corneum hydrates, water accumulates near the outer surface of the protein filaments. Polar molecules appear to pass through this immobilized water.

“Intercellular transcorneal delivery” and “intercellular transcorneal penetration” refer to the passing of a compound, typically a “hydrophobic compound” through the cells of the stratum corneum by dissolve in and diffuse through the non-aqueous lipid matrix imbibed between the protein filaments.

As used herein, the terms “transappendageal delivery” and “transappendageal penetration” refer synonymously to the shunt pathway whereby a compound traverses through the hair follicles, the sebaceous pathway of the pilosebaceous apparatus, and/or the aqueous pathway of the salty sweat glands. The transappendageal pathway is considered to be of minor importance because of its relatively smaller area (less than 0.10% of total surface). This route is of substantial relevance for the “transappendageal delivery” or “transappendageal penetration” polar, hydrophobic, and/or lipophilic compounds.

As used herein, the term “unsaturated fatty acid” refers to a fatty acid comprising one or more C═C double bonds. C═C double bonds can adopt either a “cis” or a “trans” configuration and thereby yield either a “cis unsaturated fatty acid” or a “trans unsaturated fatty acid.”

As used herein, the term “cis unsaturated fatty acid” refers to a fatty acid comprising one or more C═C double bonds in a “cis” configuration, wherein two hydrogen atoms adjacent to the double bond stick out on the same side of the C═C chain. The rigidity of a double bond freezes its conformation and, in the case of the cis isomer, causes the chain to bend and restricts the conformational freedom of the fatty acid. The more double bonds the chain has in the cis configuration, the less flexibility it has. When a chain has many cis bonds, it becomes quite curved in its most accessible conformations.

For example, oleic acid, with one double bond, has a “kink” in it, whereas linoleic acid, with two double bonds, has a more pronounced “bend.” a-Linolenic acid, with three double bonds, favors a “hooked” shape. The effect of this is that, in restricted environments, such as when fatty acids are part of a phospholipid in a lipid bilayer or triglycerides in lipid droplets, “cis” bonds limit the ability of fatty acids to be closely packed, and therefore can reduce the melting temperature of the membrane or of the fat and, thereby, “cis unsaturated fatty acids” increase cellular membrane fluidity as compared to the “trans unsaturated fatty acid” comprising the same atomic constituents/primary molecular structure.

As used herein, the term “trans unsaturated fatty acid” refers to a fatty acid comprising one or more C═C double bonds in a “trans” configuration, wherein two hydrogen atoms adjacent to the double bond lie on opposite sides of the chain. As a result, they do not cause the chain to bend much, and their shape is similar to straight saturated fatty acids. In most naturally occurring unsaturated fatty acids, each double bond has three (n-3), six (n-6), or nine (n-9) carbon atoms after it, and all double bonds have a cis configuration.

Words and phrases using the singular or plural number also include the plural and singular number, respectively. For example, terms such as “a” or “an” and phrases such as “at least one” and “one or more” include both the singular and the plural. Terms that are intended to be “open” (including, for example, the words “comprise,” “comprising,” “include,” “including,” “have,” and “having,” and the like) are to be construed in an inclusive sense as opposed to an exclusive or exhaustive sense. That is, the term “including” should be interpreted as “including but not limited to,” the term “includes” should be interpreted as “includes but is not limited to,” the term “having” should be interpreted as “having at least.”

The use of the term “or” in the claims is used to mean “and/or” unless explicitly indicated to refer to alternatives only or the alternatives are mutually exclusive, although the disclosure supports a definition that refers to only alternatives and “and/or.”

Additionally, the terms “herein,” “above,” and “below,” and words of similar import, when used in this application, shall refer to this application as a whole and not to any particular portion of the application.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

Unless specifically stated, as used herein, the term “about” in reference to a number or range of numbers is understood to mean the stated number and numbers +/−10% thereof, or 10% below the lower listed limit and 10% above the higher listed limit for the values listed for a range.

### Transdermal Delivery Formulations

In some embodiments, a transdermal delivery formulation comprises an emulsion of two immiscible liquids. An emulsion comprises a dispersed phase in a continuous phase. In some embodiments, an emulsion is stabilized by comprising one or more surfactants. In some embodiments, the liquids comprise an oil and water. In some embodiments, the oil is dispersed in the oil. In some embodiments, the oil is dispersed in the water. In some embodiments, the emulsion further comprises one or more surfactants. A surfactant is a molecule that typically comprises a hydrophilic head and a hydrophobic tail. The surfactant decreases surface tension between two liquids, stabilizing the emulsion.

In some embodiments, transdermal delivery formulations disclosed herein comprise acidified micelles and/or liposomes that encapsulate a compound, such as a nutrient or a drug. These transdermal delivery formulations are made by preparing separately (a) a transdermal accelerant comprising a weak organic acid solution having a pKa greater than 2.0 and (b) a microemulsion comprising a nonionic emulsifier, water, and a cis-unsaturated long-chain fatty acid. Those fatty acid microemulsions are combined with the acidified transdermal accelerants having a pH greater than 1.0 (typically from 1.5 to 2.5) to yield a homogenous transdermal delivery formulation comprising fatty acid micelles and/or liposomes that encapsulate one or more compound, such as a nutrient or a drug. Those micelles and/or liposomes incorporate one or more cis-unsaturated fatty acid having a 12 to 26 carbon chain that includes one or more double bond in a cis configuration. The resulting micelles and/or liposomes yield transdermal delivery formulations that exhibit ideal solubility and absorption properties in high humidity conditions, such as in a warm to hot shower or sauna.

Transdermal delivery formulations according to the present disclosure exhibit unexpected and surprising advantages over technologies that are currently available in the art for the administration of drugs, nutrients, and other compounds to a human subject or domestic, veterinary, or agricultural animal—in particular, over existing technologies of the epicutaneous administration of drugs, nutrients, and other compounds. Within certain aspects, these transdermal delivery formulations comprise a homogenous mixture of (a) a transdermal accelerant comprising a weak organic acid having a pKa greater than 2.0 and (b) a microemulsion comprising a nonionic emulsifier, water, and one or more cis-unsaturated long-chain fatty acid.

The transdermal delivery formulations disclosed herein achieve the efficient and systemic transdermal delivery/permeation of a compound through the stratum corneum and epidermis and into the bloodstream of a human subject or domestic, veterinary, or agricultural animal and facilitate the rapid diffusion of compounds comprised within the transdermal delivery formulation when applied to the outer skin surface through the stratum corneum, viable epidermis, papillary dermis, and into the microcirculation.

The viable tissue layer and the capillaries are relatively permeable, and the peripheral circulation is sufficiently rapid. Hence, diffusion through the stratum corneum is traditionally the rate-limiting step, Skin has pH of 4.2 to 5.6, solutions which have this pH range are used to avoid damage to the skin. However for a number of drugs, there may also be significant transdermal absorption at pH values at which the un-ionized form of the drug is predominant.

Once applied to the outer surface of the skin, transdermal delivery formulations traverse the skin. Skin permeability is affected by temperature, irradiation, or sun exposure. Absorption occurs through, without limitation, five mechanisms: (1) active transport, (2) passive diffusion, (3) facilitated diffusion, (4) co-transport (or secondary active transport), and (5) endocytosis. Thus, a compound within the transdermal delivery formulation moves across the skin barrier according to a number of factors. In some embodiments, formulations traverse the skin barrier via passive diffusion. Passive diffusion is calculated following Fick's first law of diffusion, which relates the diffusive flux to the gradient of the concentration. The flux goes from regions of high concentration to regions of low concentration at a rate and magnitude that is proportional to the concentration gradient

### Fick's First Law of Diffusion:

$$J = - D \frac{d φ}{dx}$$

where J is the diffusion flux, of which the dimension is the amount of substance per unit area per unit time. J measures the amount of substance that will flow through a unit area during a unit time interval. D is the diffusion coefficient or diffusivity. φ (for ideal mixtures) is the concentration, of which the dimension is the amount of substance per unit volume. x is position, the dimension of which is length. D is proportional to the squared velocity of the diffusing particles, which depends on the temperature, viscosity of the fluid, and the size of the particles according to the Stokes-Einstein relation. The driving force for the one-dimensional diffusion is the quantity −∂φ∂x, which for ideal mixtures is the concentration gradient.

In some embodiments, transdermal delivery formulations for epicutaneous administration of drugs, nutrients, or other compounds to a human subject or domestic, veterinary, or agricultural animal comprise a homogenous mixture of (a) a transdermal accelerant comprising a weak organic acid having a pKa greater than 2.0 and (b) a microemulsion comprising a nonionic emulsifier, water, and a cis-unsaturated long-chain fatty acid.

### 1. Transdermal Accelerants

The acidity of a molecule is described by the pKa. The pKa value is the negative base −10 logarithm of the acid dissociation constant (Ka) of a solution refers to acid dissociation constant (Ka) of a solution. The pH of a solution can be predicted when the analytical concentration and pKa values of all acids and bases in the solution are known; conversely, it is possible to calculate the equilibrium concentration of the acids and bases in a solution when the pH is known. These calculations find application in many different areas of chemistry, biology, medicine, and geology. For example, many compounds used for medication are weak acids or bases. The quantitative behavior of acids and bases in solution can be understood only if their pKa values are known. It measures the strength of an acid by how tightly a proton is held by a Bronsted acid. The lower the value of pKa, the stronger the acid and the greater its ability to donate its protons. Description of the acidity of a particular molecule Ka denotes the acid dissociation constant. It measures how completely an acid dissociates in an aqueous solution. The larger the value of Ka, the stronger the acid as acid largely dissociates into its ions and has lower pKa value.

The relationship between pKa and Ka is described by the following equation:

pKa=−log[Ka]

Acid dissociation constants, or pKa values, are essential for understanding many fundamental reactions in chemistry. These values reveal the deprotonation state of a molecule in a particular solvent. There is great interest in using theoretical methods to calculate the pKa values for many different types of molecules.

As used herein, the term “weak organic acid” refers to a compound that partially dissociates when dissolved in a solvent, in particular, a fatty acid microemulsion. The strength of a weak acid can be quantified in terms of a dissociation constant, defined as “pH,” which refers to the negative logarithm of the H ion concentration.

As used herein, the term “strong acids” refers to those acids that exhibit a negative pKa are that are unsuitable for use in the transdermal delivery formulations disclosed herein. Exemplary “strong acids” are presented in Table 1.

TABLE 1
Acidic Dissociation Constants (pKa) of Strong Acids

| Strong Acids | Formula | pKa (in H₂O at ~15-20° C.) | |
|---|---|---|---|
| Hydrochloric Acid | HCl | pKa | −5.9 |
| Hydrobromic Acid | HBr | pKa | −8.8 |
| Hydroiodic Acid | HI | pKa | −9.5 |
| Triflic Acid | H[CF₃SO₃] | pKa | −14 |
| Perchloric Acid | H[ClO₄] | pKa | −15 |
| Nitric Acid | HNO₃ | pKa | −1.6 |
| Sulfuric Acid | H₂SO₄ | pKa1 | −3 |

The epidermis naturally has a slight negative charge, while interior tissues carry a slight positive charge. This potential difference induces movement of ions through the layers in a unidirectional manner. Under normal conditions, positive ions, for example, sodium (Na+) are transported toward the outer layers of skin, and negative ions, such as chloride (Cl−) are transported towards the inner layers. In some embodiments described herein, application of ionic solutions as with a weak acid leverage this charge difference to effect transport across the skin. Transdermal accelerants according to the present disclosure are based upon the observation that weak organic acids, particularly, those having a pKa at or below the pH of the skin (presented in Table 2), when used in combination with a fatty acid microemulsion, as presented herein, greatly enhance skin permeability and delivery of drugs, nutrients, and other compounds through the skin and into the bloodstream.

TABLE 2
Median Acidic Dissociation Constants (pKa) of Weak Organic Acids

| | | | | Median pKa |
|---|---|---|---|---|
| | | pKa (in H₂O at | | (in H₂O at |
| Weak Organic Acid | Structure | ~15-20° C.) | | ~15-20° C.) |
| Succinic Acid | 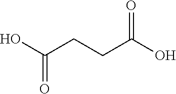 | pKa1 pKa2 | 4.2 5.6 | 4.90 |
| Acetic Acid | 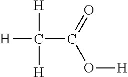 | pKa1 | 4.756 | 4.756 |
| Gallic Acid | 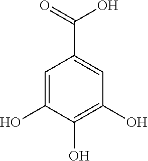 [SMILES: O=C(O)c1cc(O)c(O)c(O)c1] | pKa1 pKa2 pKa3 | 3.13 8.84 12.40 | 4.40 |
| Citric Acid | 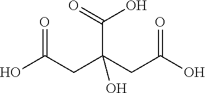 [SMILES: O=C(O)CC(O)(CC(=O)O)C(=O)O] | pKa1 pKa2 pKa3 | 3.128 4.76 6.40 | 4.76 |
| Malonic Acid | 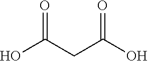 | pKa1 pKa2 pKa2 | 2.83 5.69 5.6 | 4.26 |
| Malic Acid | 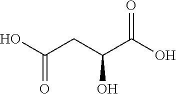 | pKa1 pKa2 | 3.40 5.20 | 3.99 |
| Maleic Acid | 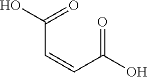 | pKa1 pKa2 | 1.90 6.07 | 3.99 |
| Lactic Acid | 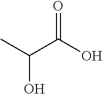 | pKa1 | 3.86 | 3.86 |
| Formic Acid | 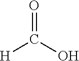 | pKa1 | 3.751 | 3.751 |
| Fumaric Acid | 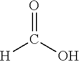 | pKa1 | 3.03 | 3.74 |
| | | pKa2 | 4.44 | |
| Tartaric Acid | 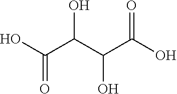 | pKa1 pKa2 | 2.89 4.40 | 3.65 |
| Oxalic Acid | 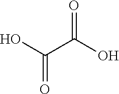 | pKa1 pKa2 pKa2 pKa2 | 1.25 4.14 5.20 6.07 | 2.7 |

Representative transdermal delivery formulations disclosed herein comprise a transdermal accelerant employing a weak organic acid solution that comprises one or more weak organic acids. In some embodiments, the one or more weak organic acids comprises a mono, di or tri carbonic acid of chain lengths (R) between 1-16. In some embodiments, the weak organic acid comprises mono or poly hydroxy moieties of 0-14. In some embodiments, the organic acid derives from a linear, branched, or cyclic structure. In some embodiments, the structure is saturated or unsaturated. In some embodiments, the one or more weak organic acids comprises lactic acid, acetic acid, formic acid, citric acid, oxalic acid, gallic acid, malic acid, maleic acid, malonic acid, succinic acid, tartaric acid, fumaric acid, or any combination thereof. In some embodiments, apple cider vinegar is provided as a source of acetic acid. Apple cider vinegar is generally about 5% acetic acid, about 94% water, and about 1% carbohydrates.

Transdermal accelerants suitable for use in the manufacture of these transdermal delivery formulations comprise one or more weak organic acids. In some embodiments, a weak organic acid has a median pKa of from 2.0 to 6.0, from 3.0 to 5.5, from 4.0 to 5.0, or about 4.7. In some embodiments, the one or more weak organic acids comprises a mono, di or tri carbonic acid of chain length (R) between 1-16. In some embodiments, the weak organic acid comprises mono or poly hydroxy moieties of 0-14. In some embodiments, the organic acid derives from a linear, branched, or cyclic structure. In some embodiments, the structure is saturated or unsaturated. In some embodiments, the one or more weak organic acids comprises lactic acid, acetic acid, formic acid, citric acid, oxalic acid, gallic acid, malic acid, maleic acid, malonic acid, succinic acid, tartaric acid, fumaric acid, or any combination thereof. Exemplified herein are transdermal accelerants wherein said weak organic acid is citric acid or acetic acid or a combination of both citric acid and acetic acid.

### 2. Fatty Acid Microemulsions

Transdermal delivery formulations for epicutaneous administration of a drug, nutrient, or other compound to a human subject or domestic, veterinary, or agricultural animal comprise: a homogenous mixture of (a) a transdermal accelerant comprising a weak organic acid (as described herein above) and (b) a microemulsion comprising a nonionic emulsifier, water, and an unsaturated long-chain fatty acid.

The fatty acid microemulsions according to the present disclosure are based upon the observation that certain unsaturated fatty acids, particularly, those having a C12-C26 carbon chain or a C14-C26 carbon chain or a C16-C26 carbon chain when used in combination with a nonionic emulsifier, particularly, those selected from the group consisting of lecithin, carboxylmethylcellulose, a sorbitan ester, and a polysorbate greatly enhance skin permeability and delivery of drugs, nutrients, and other compounds through the skin and into the bloodstream.

As used herein, the terms “non-ionic surfactant,” “non-ionic emulsifier,” and non-ionic detergent” refer collectively to compounds that stabilize an emulsion by reducing the oil-water interface tension. Non-ionic surfactants, emulsifiers, and detergents are typically amphiphilic compounds having both a polar, hydrophilic, and water-soluble portion and a non-polar, hydrophobic, and lipophilic portion. Non-ionic surfactants, emulsifiers, and detergents employed in the presently disclosed transdermal delivery formulations include lecithin, carboxylmethylcellulose, sorbitan esters, and polysorbates, including polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and polyoxyethylene (20) sorbitan monooleate (polysorbate 80).

Polysorbate 80, otherwise known as Tween, is a yellow/golden-colored viscous liquid used as an emulsifier or surfactant in foods, medicines, skincare products, and vaccines. Primarily used to solubilize proteins and is widely used in injectable medications, vaccines.

Polysorbate 80 NF (polyoxyethylene sorbitan monooleate) is derived from RSPO palm oil. It is a non-toxic, nonionic surfactant/emulsifier and a water-soluble yellowish liquid used as a dispersing agent which allows oil and water to mix without the use of alcohol. Polysorbate 80 is a complex mixture consisting of a series of esters and etherates synthesized separately by oleic acid and ethylene oxide with a two-core matrix of sorbitan (Fatty acids in olive oil that are combined with sorbitol. It is plant-derived). Polysorbate 80 helps solubilize ingredients and is considered safe by the FDA for use vitamin and vitamin-mineral preparations which can contain up to 475 milligrams per daily serving of polysorbate 80 (FDA Food Additive Status List).

Polysorbate 80 has been shown to inhibit reflex pumps involved in the blood-brain barrier (BBB) (e.g., Polysorbate-80 modified neurotoxin microparticles can transport across the BBB). Polysorbate 80 can promote trimethylolpropane phosphate (TMPP) distribution in the brain by increasing drug systemic absorption and then enhanced passive transport of TMPP through the BBB, with the nose-to-brain direct transport percentage decreased to some extent.

The geometric differences between the various types of unsaturated fatty acids, as well as between saturated and unsaturated fatty acids, play an important role in biological processes, and in the construction of biological structures (such as cell membranes). Suitable fatty acids for use in the transdermal delivery formulations disclosed herein include unsaturated long-chain fatty acids comprising a chain of from 12 to 26 carbons and one or more double bonds in a cis configuration. As depicted in Table 3, unsaturated long-chain fatty acids comprising a single double bond in a cis configuration (e.g., oleic acid) adopt a “kink” conformation while unsaturated long-chain fatty acids comprising two double bonds in a cis configuration (e.g., linoleic acid) adopt a “bend” conformation, and unsaturated long-chain fatty acids comprising three double bonds in a cis configuration (e.g., Linolenic acid) adopt a “hook” conformation. The effect of this is that, in restricted environments, such as when fatty acids are part of a phospholipid in a lipid bilayer or triglycerides in lipid droplets, cis unsaturated fatty acids increase cellular membrane fluidity by limiting the ability of fatty acids to be closely packed, and thereby reduce the melting temperature of the membrane or of the fat.

In contrast, because the adjacent two hydrogen atoms flanking trans double bond lie on opposite sides of the chain, trans unsaturated long-chain fatty acids comprising a chain of from 12 to 26 carbons and one or more double bonds in a trans configuration exhibit little structural alteration as compared to the corresponding fully-saturated long-chain fatty acid. The effect of this is that, in restricted environments, such as when fatty acids are part of a phospholipid in a lipid bilayer or triglycerides in lipid droplets, trans unsaturated fatty acids decrease cellular membrane fluidity by enhancing the ability of fatty acids to be closely packed, and thereby increase the melting temperature of the membrane or of the fat. See, e.g., Table 3, stearic acid (C18:0 saturated), elaidic acid (C18:1 in trans), and linolelaidic acid (C18:2 in trans).

Thus, disclosed herein are transdermal delivery formulations comprising fatty acid microemulsions comprising one or more 12 to 26 carbon unsaturated long-chain fatty acids. In some embodiments, the one or more 12 to 26 carbon unsaturated long-chain fatty acids comprise adrenic acid, arachidonic acid, arachidic acid, behenic acid, brassidic acid, cervonic acid, cis-vaccenic acid, dihomo-y-linolenic acid, docosadienoic acid, eicosadienoic acid, eicosapentaenoic acid, eicosatetraenoic acid, eicosenoic acid, elaidic acid, erucic acid, gadoleic acid, gondoic acid, herring acid, lauric acid, lignoceric acid, linoleic acid, linolelaidic acid, margaric acid, margoleic acid, mead acid, myristoleic acid, nervonic acid, oleic acid, ozubondo acid, palmitic acid, palmitoleic acid, trans-palmitoleic acid, paullinic acid, petroselinic acid, pinolenic acid, sapienic acid, sardine acid, stearic acid, stearidonic acid, tetracosapentaenoic acid, α-linolenic acid, γ-linolenic acid, or any combination thereof. Exemplified herein are transdermal delivery formulations wherein the 12 to 26 carbon unsaturated long-chain fatty acid is oleic acid or linoleic acid as well as transdermal delivery formulations wherein the 12 to 26 carbon unsaturated long-chain fatty acid comprises a combination oleic acid and linoleic acid.

TABLE 3
Structural and Physical Properties of Unsaturated Long-chain Fatty Acids

| | | | Double | |
|---|---|---|---|---|
| | Naturally | Carbons: | Bonds | |
| | Occurring | Double | (cis or | |
| Fatty Acid | Source | Bonds | trans) | Secondary Structure |
| Adrenic acid | naturally occurring in humans | C22:4 | cis | 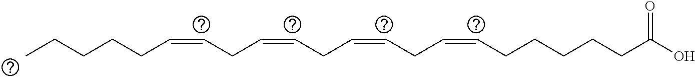 [SMILES: CCCCC/C=C\C/C=C\C/C=C\C/C=C\CCCCCC(=O)O] |
| Arachidonic acid | Various Plant | C20:4 | cis | 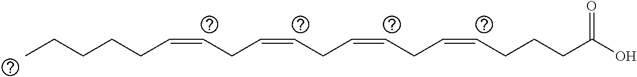 [SMILES: CCCCC/C=C\C/C=C\C/C=C\C/C=C\CCCC(=O)O] |
| Arachidic acid | Various Plant | C20:0 | saturated | 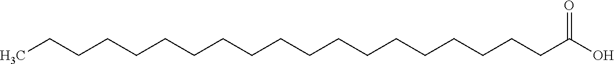 [SMILES: CCCCCCCCCCCCCCCCCCCC(=O)O] |
| Behenic acid | drumstick tree (Moringa oleifera) | C22:0 | saturated | 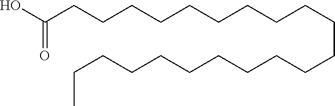 [SMILES: CCCCCCCCCCCCCCCCCCCCCC(=O)O] |
| Brassidic acid | rapeseed plant oil extracts and canola oil. | C22:1 | trans | 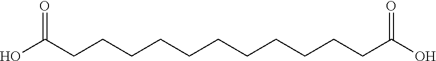 [SMILES: O=C(O)CCCCCCCCCCCC(=O)O] |
| Cervonic acid | maternal milk, fish oil | C22:6 | cis | 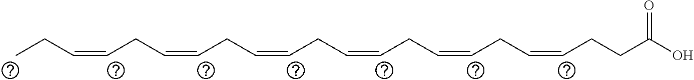 [SMILES: CC/C=C\C/C=C\C/C=C\C/C=C\C/C=C\C/C=C\CCC(=O)O] |
| Cis- Vaccenic acid | milk, butter, and yogurt. | C18:1 | cis | 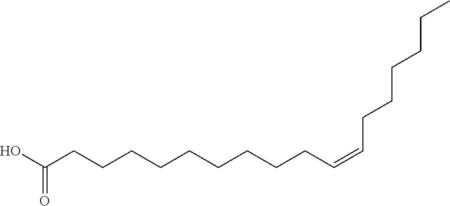 [SMILES: CCCCCC/C=C\CCCCCCCCCC(=O)O] |
| Dihomo-γ- Linolenic acid | only in trace amounts in animal products | C20:3 | cis | 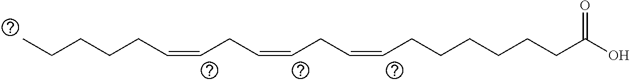 [SMILES: CCCCC/C=C\C/C=C\C/C=C\CCCCCCC(=O)O] |
| Docosadienoic acid | mushroom | C22:2 | cis | 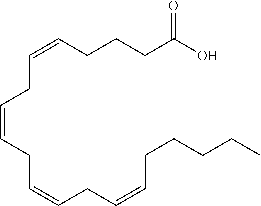 [SMILES: CCCCC/C=C\C/C=C\C/C=C\C/C=C\CCCC(=O)O] |
| Eicosadienoic acid | Salvia hispanica, Arbacia punctulata | C20:2 | cis | 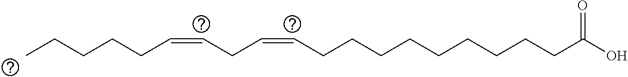 [SMILES: CCCCC/C=C\C/C=C\CCCCCCCCCC(=O)O] |
| Eicosapentaenoic acid | cod liver, herring, mackerel, salmon, menhaden and sardine | C20:5 | cis | 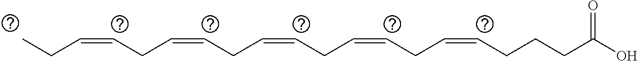 [SMILES: CC/C=C\C/C=C\C/C=C\C/C=C\C/C=C\CCCC(=O)O] |
| Eicosatetraenoic acid | Mortierella alpina 1S-4 is a fungus | C20:4 | cis | 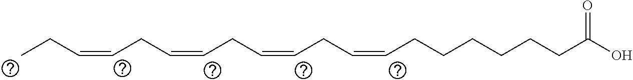 [SMILES: CC/C=C\C/C=C\C/C=C\C/C=C\CCCCCCC(=O)O] |
| Eicosenoic acid | krill drying oil | C18:3 | cis | 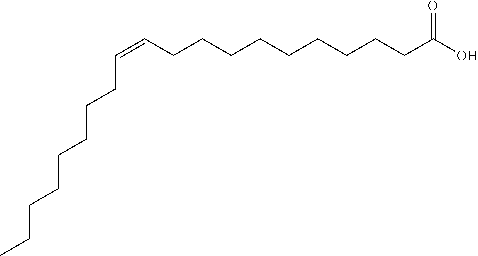 [SMILES: CCCCCCCC/C=C\CCCCCCCCCC(=O)O] |
| Elaidic acid | hydrogenated vegetable oil | C18:1 | trans | 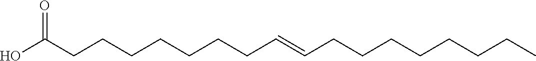 [SMILES: CCCCCCCC/C=C/CCCCCCCC(=O)O] |
| Erucic acid | wallflower seed, mustard oil | C22:1 | cis | 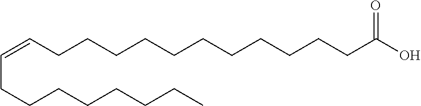 [SMILES: CCCCCCCC/C=C\CCCCCCCCCCCC(=O)O] |
| Gadoleic acid | Cod Liver Oil, marine animal oils | C20:1 | cis | 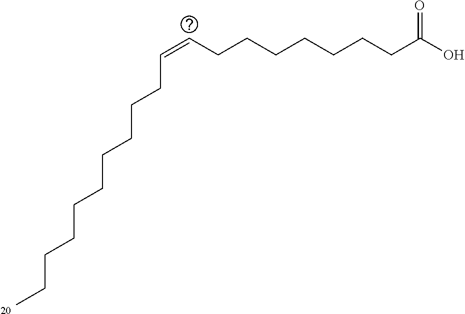 [SMILES: CCCCCCCCCC/C=C\CCCCCCCC(=O)O] |
| Gondoic acid | jojoba oil (edible but non- caloric and non- digestible) | C20:1 | cis | 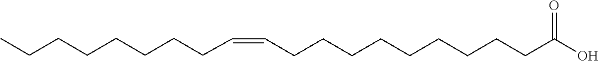 [SMILES: CCCCCCCC/C=C\CCCCCCCCCC(=O)O] |
| Herring acid | herring oil | C24:6 | cis | 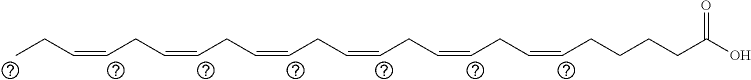 [SMILES: CC/C=C\C/C=C\C/C=C\C/C=C\C/C=C\C/C=C\CCCCC(=O)O] |
| Lauric acid | coconut oil | C12:0 | saturated | 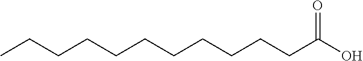 |
| Lignoceric acid | peanut oil | C24:0 | saturated | 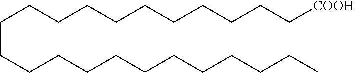 [SMILES: CCCCCCCCCCCCCCCCCCCCCCCC(=O)O] |
| Linoleic acid | peanut oil, chicken fat, olive oil | C18:2 | cis | 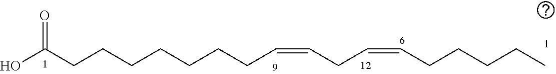 [SMILES: CCCCC/C=C\C/C=C\CCCCCCCC(=O)O] |
| Linolelaidic acid | partially hydrogenated vegetable oils | C18:2 | trans | 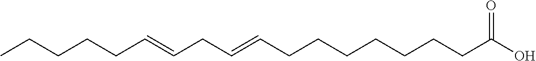 [SMILES: CCCCC/C=C/C/C=C/CCCCCCCC(=O)O] |
| Margaric acid | fat, milkfat of ruminants | C17:0 | saturated | 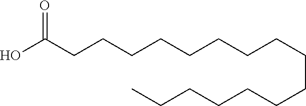 [SMILES: CCCCCCCCCCCCCCCCC(=O)O] |
| Margoleic acid | olive oil | C17:1 | cis | 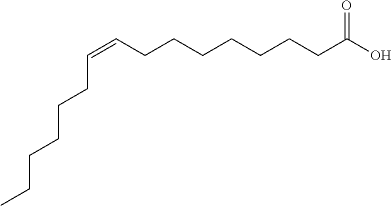 [SMILES: CCCCCC/C=C\CCCCCCCC(=O)O] |
| Mead acid | cartilage | C20:3 | cis | 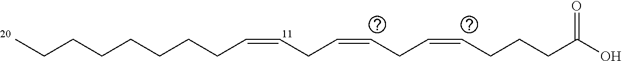 [SMILES: CCCCCCCC/C=C\C/C=C\C/C=C\CCCC(=O)O] |
| Myristoleic acid | Saw palmetto | C14:1 | cis | 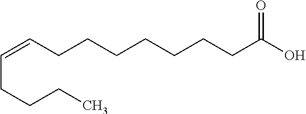 [SMILES: CCCC/C=C\CCCCCCCC(=O)O] |
| Nervonic acid | King salmon, flaxseed, sockeye salmon, sesame seed, macadamia nuts | C24:1 | cis | 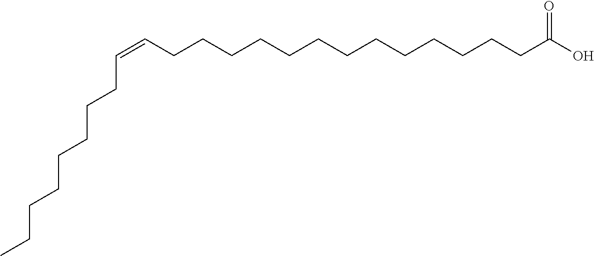 [SMILES: CCCCCCCC/C=C\CCCCCCCCCCCCCC(=O)O] |
| Oleic acid | olive oil, pecan oil, canola oil | C18:1 | cis | 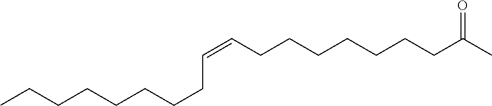 [SMILES: CCCCCCCC/C=C\CCCCCCCC(C)=O] |
| Ozubondo acid | plants and mice | C22:5 | cis | 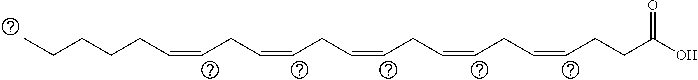 [SMILES: CCCCC/C=C\C/C=C\C/C=C\C/C=C\C/C=C\CCC(=O)O] |
| Palmitic acid | olive oil | C16:0 | saturated | 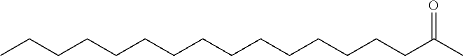 [SMILES: CCCCCCCCCCCCCCCC(C)=O] |
| Palmitoleic acid | macadamia nuts | C16:1 | cis | 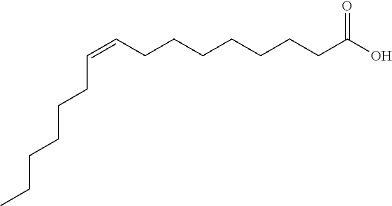 [SMILES: CCCCCC/C=C\CCCCCCCC(=O)O] |
| trans- Palmitoleic acid | milk, cheese, yogurt, and butter. | C16:1 | trans | 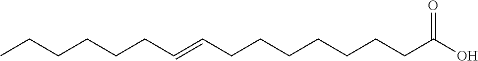 [SMILES: CCCCCC/C=C/CCCCCCCC(=O)O] |
| tran- Vaccenic acid | Agaricus blazei, Allamanda cathartica, | C18:1 | trans | 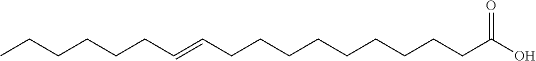 [SMILES: CCCCCC/C=C/CCCCCCCCCC(=O)O] |
| Paullinic acid | guarana | C20:1 | cis | 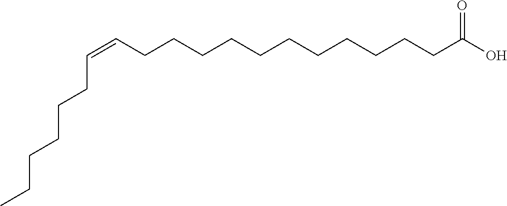 [SMILES: CCCCCC/C=C\CCCCCCCCCCCC(=O)O] |
| Petroselinic acid | vegetable oil of Coriandrum sativum fruits | C18:1 | cis | 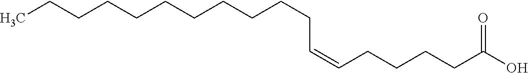 [SMILES: CCCCCCCCCCC/C=C\CCCCC(=O)O] |
| Pinolenic acid | pine nut oil | C18:3 | cis | 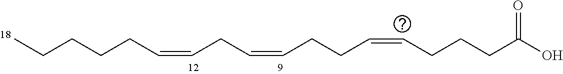 [SMILES: CCCCC/C=C\C/C=C\CC/C=C\CCCC(=O)O] |
| Sapienic acid | human skin | C16:1 | cis | 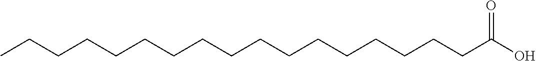 [SMILES: CCCCCCCCCCCCCCCCCC(=O)O] |
| Sardine acid | sardines | C22:5 | cis | 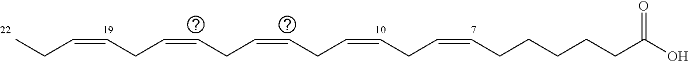 [SMILES: CC/C=C\C/C=C\C/C=C\C/C=C\C/C=C\CCCCCC(=O)O] |
| Stearic acid | olive oil | C18:0 | saturated | 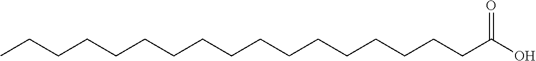 [SMILES: CCCCCCCCCCCCCCCCCC(=O)O] |
| Stearidonic acid | seed oils of hemp, blackcurrant, corn gromwell | C18:4 | cis | 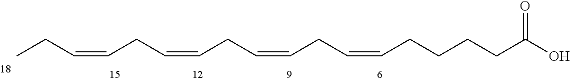 [SMILES: CC/C=C\C/C=C\C/C=C\C/C=C\CCCCC(=O)O] |
| Tetracosa- pentaenoic acid | salmon | C24:5 | cis | 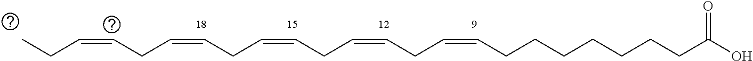 [SMILES: CC/C=C\C/C=C\C/C=C\C/C=C\C/C=C\CCCCCCCC(=O)O] |
| α-Linolenic acid | flaxseeds, chia seeds, walnuts, lin seed | C18:3 | cis | 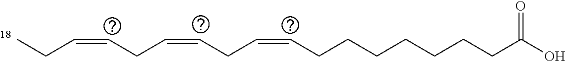 [SMILES: CC/C=C\C/C=C\C/C=C\CCCCCCCC(=O)O] |
| γ-Linolenic acid | borage oil, black currant oil, evening primrose oil and safflower oil | C18:3 | cis | 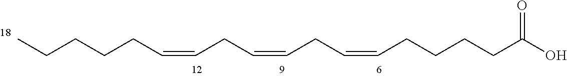 [SMILES: CCCCC/C=C\C/C=C\C/C=C\CCCCC(=O)O] |

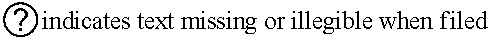

In related embodiments, the presently disclosed transdermal delivery formulations for epicutaneous administration of a therapeutic agent, drug, nutrient, or other compound to a human subject; veterinary animal; domesticated or undomesticated animal, plant or insect; or agricultural animal, plant, or insect comprise: a homogenous mixture of (a) a transdermal accelerant comprising a weak organic acid (as described herein above) and (b) a microemulsion comprising a nonionic emulsifier, water, and plant or animal oil.

Suitable plant or animal oils for use in the microemulsions disclosed herein include vegetable oils, nut oils, seed oils or animal oils comprising from 50%-100%, or from 60%-90%, or from 70%-90%, or from 80%-90% of the total unsaturated fatty acid content as long-chain unsaturated fatty acids comprising one or more cis double bonds. Representative suitable plant or animal oils are selected from the group consisting of macadamia oil, maracuja (passion fruit) oil, safflower oil, sunflower oil, olive oil, avocado oil, canola oil, coconut oil, corn oil, cottonseed oil, flaxseed/linseed oil, grape seed oil, hemp seed oil, palm oil, peanut oil, rice bran oil, sesame oil, soybean oil, Brazil nut oil, almond oil, walnut oil, pecan oil, jojoba oil, chia seed oil, wallflower seed, mustard oil, borage oil, black currant oil, evening primrose oil, chicken fat, cartilage oil, cod liver oil, herring oil, mackerel oil, salmon oil, menhaden oil, sardine oil, or any combination thereof.

Exemplified herein are transdermal delivery formulations comprising one or more plant oils as presented in Table 4 “Plant and Animal Oil Unsaturated Fatty Acid Profiles.” In some embodiments, plant or animal oils comprise macadamia oil, maracuja (passion fruit) oil, safflower oil, sunflower oil, olive oil, and almond oil, fish oil, chicken fat, or any combination thereof. In certain aspects, the transdermal delivery formulation comprises macadamia oil or maracuja (passion fruit) oil. In other aspects, the plant or animal oil comprises a combination of macadamia oil and maracuja (passion fruit) oil.

TABLE 4
Plant and Animal Oil Unsaturated Fatty Acid Profiles

| | | | |
|---|---|---|---|
| | Average | Primary | |
| | Fatty | Structure | |
| | Acid | (Carbons:Dou- | Double Bonds |
| Fatty Acid | Content | ble Bonds) | (cis or trans) |

Macadamia Oil

| | | | |
|---|---|---|---|
| Palmitoleic Acid | 19.5% | C16:1 | cis Omega-7 Fatty Acid |
| Oleic Acid | 61.0% | C18:1 | cis Omega-9 Fatty Acid |
| Linoleic Acid | 2.2% | C18:2 | cis Essential omega-6 |
| | | | Fatty Acid |
| Eicosenoic | 2.8% | C20:1 | cis Omega-3 and -6 |
| | | | Fatty Acids |
| Total % Unsaturated | 85.5% | | |
| Fatty Acids | | | |

Salmon Oil

| | | | |
|---|---|---|---|
| Palmitoleic Acid | 8.7% | C16:1 | cis Omega-7 Fatty Acid |
| Oleic Acid | 18.6% | C18:1 | cis Omega-9 Fatty Acid |
| Linoleic Acid | 1.2% | C18:2 | cis Essential omega-6 |
| | | | Fatty Acid |
| Eicosenoic | 8.4% | C20:1 | cis Omega-3 and -6 |
| | | | Fatty Acids |
| Erucic | 5.5% | C22:1 | cis Omega-3 and -6 |
| | | | Fatty Acid |
| Eicosapentaenoic | 5.0% | C20:5 | cis Essential omega-3 |
| | | | Fatty Acid |
| Docosahexaenoic | 13.8% | C22:6 | cis Essential omega-3 |
| | | | Fatty Acid |
| Stearidonic | 2.1% | C18:4 | cis Essential omega-3 |
| | | | Fatty Acid |
| Docosapentaenoic | 2.9% | C22:5 | cis Essential omega-3 |
| | | | Fatty Acid |
| Linolenic | 0.6% | C18:3 | cis Essential omega-3 |
| | | | Fatty Acid |
| Arachidonic | 0.9% | C20:4 | cis Essential omega-3 |
| | | | Fatty Acid |
| Total % Unsaturated | 74.7% | | |
| Fatty Acids | | | |

Sardine Oil

| | | | |
|---|---|---|---|
| Palmitoleic Acid | 9.2% | C16:1 | cis Omega-7 Fatty Acid |
| Oleic Acid | 11.4% | C18:1 | cis Omega-9 Fatty Acid |
| Linoleic Acid | 0.9% | C18:2 | cis Essential omega-6 |
| | | | Fatty Acid |
| Eicosenoic | 3.2% | C20:1 | cis Omega-3 and -6 |
| | | | Fatty Acids |
| Erucic | 3.8% | C22:1 | cis Omega-3 and -6 |
| | | | Fatty Acid |
| Eicosapentaenoic | 16.9% | C20:5 | cis Essential omega-3 |
| | | | Fatty Acid |
| Docosahexaenoic | 21.9% | C22:6 | cis Essential omega-3 |
| | | | Fatty Acid |
| Stearidonic | 2.0% | C18:4 | cis Essential omega-3 |
| | | | Fatty Acid |
| Docosapentaenoic | 2.5% | C22:5 | cis Essential omega-3 |
| | | | Fatty Acid |
| Linolenic | 1.3% | C18:3 | cis Essential omega-3 |
| | | | Fatty Acid |
| Arachidonic | 1.6% | C20:4 | cis Essential omega-3 |
| | | | Fatty Acid |
| Total % Unsaturated | 74.7% | | |
| Fatty Acids | | | |

Tallow

| | | | |
|---|---|---|---|
| Palmitoleic Acid | 3.0% | C16:1 | cis Omega-7 Fatty Acid |
| Oleic Acid | 47.0% | C18:1 | cis Omega-9 Fatty Acid |
| Linoleic Acid | 3.0% | C18:2 | cis Essential omega-6 |
| | | | Fatty Acid |
| Linolenic | 1.0% | C18:3 | cis Omega-9 Fatty Acids |
| Total % Unsaturated | 54.0% | | |
| Fatty Acids | | | |

Herring Oil

| | | | |
|---|---|---|---|
| Palmitoleic Acid | 7.5% | C16:1 | cis Omega-7 Fatty Acid |
| Oleic Acid | 12.9% | C18:1 | cis Omega-9 Fatty Acid |
| Linoleic Acid | 1.1% | C18:2 | cis Essential omega-6 |
| | | | Fatty Acid |
| Eicosenoic | 15.1% | C20:1 | cis Omega-3 and -6 |
| | | | Fatty Acids |
| Erucic | 22.0% | C22:1 | cis Omega-3 and -6 |
| | | | Fatty Acid |
| Eicosapentaenoic | 6.8% | C20:5 | cis Essential omega-3 |
| | | | Fatty Acid |
| Docosahexaenoic | 5.8% | C22:6 | cis Essential omega-3 |
| | | | Fatty Acid |
| Stearidonic | 1.4% | C18:4 | cis Essential omega-3 |
| | | | Fatty Acid |
| Docosapentaenoic | 0.8% | C22:5 | cis Essential omega-3 |
| | | | Fatty Acid |
| Linolenic | 0.7% | C18:3 | cis Essential omega-3 |
| | | | Fatty Acid |
| Arachidonic | 0.3% | C20:4 | cis Essential omega-3 |
| | | | Fatty Acid |
| Total % Unsaturated | 74.4% | | |
| Fatty Acids | | | |

### 3. Composition of Exemplary Transdermal Delivery Formulation

In some embodiments, a transdermal delivery formulation as provided herein comprises a transdermal accelerant and a carrier. In some embodiments, the transdermal accelerant and the carrier are present in the transdermal delivery formulation at a ratio of about 8:1, 7:1, 6:1, 5:1, 4:1, 3:1, 2:1, 1:1, 1:2, 1:3, 1:4, 1:5, 1:6, 1:7, or 1:8 by weight. In some embodiments, the transdermal delivery formulation comprises about 2%, about 3%, about 4%, about 4.5%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, or about 80% weight/weight (w/w) of the transdermal accelerant. In some embodiments, a transdermal delivery formulation of about 3100 g comprises about 200 g, about 300 g, about 400 g, about 500 g, about 600 g, about 700 g, about 800 g, about 900 g, about 1000 g, about 1100 g, about 1200 g, about 1300 g, about 1400 g, about 1500 g, about 1600 g, about 1700 g, about 1800 g, about 1900 g, or about 2050 g of the transdermal accelerant.

In some embodiments, the transdermal delivery formulation comprises about 10%, about 15%, about 20%, about 25%, about 30%, about 35%, about 40%, about 45%, about 50%, about 55%, about 60%, about 65%, about 70%, about 75%, or about 80% weight/weight (w/w) of the carrier. In some embodiments, a transdermal delivery formulation of about 3100 g comprises about 1000 g, about 1100 g, about 1200 g, about 1300 g, about 1400 g, about 1500 g, about 1600 g, about 1700 g, about 1800 g, about 1900 g, about 2000 g, about 2100 g, about 2200 g, about 2300 g, or about 2400 g of the carrier.

In some embodiments, the transdermal accelerant comprises one or more weak organic acids. In some embodiments, the transdermal accelerant comprises 2, 3, 4, 5, 6, 7, 9, 9 or 10 weak organic acids. In some embodiments, the transdermal accelerant comprises two or more weak organic acids. In some embodiments, the transdermal accelerant comprises 2 weak organic acids at a ratio by weight of about 1:1, 1:2, 1:3, 1:4, 1:5, 1:6, 1:7, 1:9, or 1:10 relative to each other. In some embodiments, the transdermal accelerant comprises 3 weak organic acids in equal amounts by weight. In some embodiments, the transdermal accelerant comprises 3 weak organic acids at a ratio by weight of X:Y:Z, wherein X=1-5, Y=1-6, and Z=1-6. In some embodiments, the ratio is about 1:1:1, 1:1:2, 1:1:3, 1:1:4, 1:1:5, 1:1:6, 1:2:2, 1:2:3, 1:2:4, 1:2:5, 1:2:6, 1:3:3, 1:3:4, 1:3:5, 1:3:6, 1:4:4, 1:4:5, 1:4:6, 1:5:5, 1:5:6, 1:6:6, 2:2:3, 2:2:4, 2:2:5, 2:2:6, 2:3:3, 2:3:4, 2:3:5, 2:3:6, 2:4:4, 2:4:5, 2:4:6, 2:5:5, 2:5:6, 2:6:6, 3:3:4, 3:3:5, 3:3:6, 3:4:4, 3:4:5, 3:4:6, 3:5:5, 3:5:6, 3:6:6, 4:4:5, 4:4:6, 4:5:5, 4:5:6, 4:6:6, 5:5:6, or 5:6:6. In some embodiments, the transdermal accelerant comprises about 60%, about 65%, about 70%, about 75%, about 80%, about 85%, about 90%, about 95%, about 97%, about 99% (w/w) of one or more weak organic acids. In some embodiments, the transdermal delivery formulation comprises about 5% (w/w), about 10%, about 15%, about 20%, about 25%, about 30%, about 35%, about 40%, about 45%, about 50%, about 55%, about 60%, about 65%, about 70% of one or more weak organic acids. In some embodiments, the weak organic acids comprise citric acid and apple cider vinegar.

In some embodiments, a transdermal accelerant of about 1050 g comprises one or more weak organic acids by weight in a range of about 400 g to about 1050 g. In some embodiments, a transdermal accelerant of about 1050 g comprises from about 400 g to about 1500 g, in some embodiments, a transdermal accelerant of about 1050 g comprises about 400 g, about 500 g, about 600 g, about 700 g, about 800 g, about 900 g, about 1000 g of one or more weak organic acids.

In some embodiments, the weak organic acids comprise citric acid and apple cider vinegar. In some embodiments, a transdermal accelerant of about 1050 g comprises from about 100 g to about 700 g, from about 200 g to about 500 g, from about 300 g to about 400 g citric acid. In some embodiments, a transdermal accelerant of about 1050 g comprises about 100 g, about 150 g, about 200 g, about 250 g, about 300 g, about 350 g, about 400 g, about 450 g, about 500 g, about 550 g, about 600 g, about 650 g, about 700 g citric acid. In some embodiments, a transdermal accelerant of about 1050 g comprises from about 300 g to about 1300 g, from about 400 g to about 900 g, from about 600 g to about 700 g apple cider vinegar. In some embodiments, a transdermal accelerant of about 1050 g comprises about 300 g, about 350 g, about 400 g, about 450 g, about 500 g, about 550 g, about 600 g, about 650 g, about 700 g, about 750 g, about 800 g, about 850 g, about 900 g, about 950 g, about 1000 g, about 1100 g, about 1200 g, about 1300 g apple cider vinegar. In some embodiments, a transdermal accelerant of about 1050 g comprises about 374 g citric acid and about 640 g apple cider vinegar.

In some embodiments, the transdermal accelerant further comprises a nitrate source. In some embodiments the nitrate source is a plant-based nitrate source. In some embodiments, the plant-based nitrate source comprises arugula, spinach, leafy green vegetables, beetroot, or any combination thereof. In some embodiments, the plant-based nitrate source is a dried powder. In some embodiments, the transdermal accelerant comprises about 2.0%, about 2.5%, about 3.0%, about 3.5%, about 4.0%, about 4.5%, about 5.0%, about 5.5%, about 6.0%, about 6.5%, about 7.0%, about 7.5%, or about 8.0% w/w of a nitrate source. In some embodiments, a transdermal accelerant of about 1050 g comprises from about 10 g to about 100 g, from about 30 g to about 70 g, from about 40 g to about 50 g of a nitrate source. In some embodiments, a transdermal accelerant of about 1050 g comprises about 10 g, about 15 g, about 20 g, about 25 g, about 30 g, about 35 g, about 40 g, about 45 g, about 50 g, about 55 g, about 60 g, about 65 g, about 70 g, about 75 g, about 80 g, about 85 g, about 90 g, about 95 g, or about 100 g of a nitrate source. In some embodiments, the transdermal delivery formulation comprises about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0% w/w of a nitrate source. In some embodiments, the plant-based nitrate source is beetroot powder. In some embodiments, a transdermal accelerant of about 1050 g comprises 44.4 g beetroot powder.

In some embodiments, the carrier as provided herein comprises at least one emulsifier, at least one oil comprising a long-chain fatty acid, and water. In some embodiments, the carrier comprises equal parts by weight of the emulsifier, oil, and water. In some embodiments, the carrier comprises an emulsifier, an oil, and water at a ratio by weight of X:Y:Z, wherein X=1-5, Y=1-6, and Z=1-6. In some embodiments, about 1:1:1, 1:1:2, 1:1:3, 1:1:4, 1:1:5, 1:1:6, 1:2:2, 1:2:3, 1:2:4, 1:2:5, 1:2:6, 1:3:3, 1:3:4, 1:3:5, 1:3:6, 1:4:4, 1:4:5, 1:4:6, 1:5:5, 1:5:6, 1:6:6, 2:2:3, 2:2:4, 2:2:5, 2:2:6, 2:3:3, 2:3:4, 2:3:5, 2:3:6, 2:4:4, 2:4:5, 2:4:6, 2:5:5, 2:5:6, 2:6:6, 3:3:4, 3:3:5, 3:3:6, 3:4:4, 3:4:5, 3:4:6, 3:5:5, 3:5:6, 3:6:6, 4:4:5, 4:4:6, 4:5:5, 4:5:6, 4:6:6, 5:5:6, or 5:6:6.

In some embodiments, the carrier comprises about 5% (w/w), about 10%, about 15%, about 20%, about 25%, about 30%, about 35%, about 40%, about 45%, about 50%, about 55%, about 60%, about 65%, about 70% of at least one oil comprising a long-chain fatty acid. In some embodiments, the carrier comprises two oils comprising a long-chain fatty acid. In some embodiments, the carrier comprises two oils comprising a long-chain fatty acid in a ratio by weight of about 1:2, 1:3, 1:4, 1:5, 1:6, 1:7, 1:9, 1:10, 1:100 or 1:1000 relative to each other.

In some embodiments, a carrier of about 2050 g comprises about 300-1500 g one or more oils comprising a long-chain fatty acid. In some embodiments, a carrier of about 2050 g comprises about 300 g, about 400 g, about 500 g, about 600 g, about 700 g, about 800 g, about 900 g, about 1000 g, about 1100 g, about 1200 g, about 1300 g, about 1400 g, about 1500 g one or more oils comprising a long-chain fatty acid.

In some embodiments, the one or more oils comprise macadamia oil and maracuja oil. In some embodiments, a carrier of about 2050 g comprises from about 100 to about 600 g, from about 200 g to about 500 g, from about 300 g to about 400 g macadamia oil. In some embodiments, a carrier of about 2050 g comprises about 100 g, about 150 g, about 200 g, about 250 g, about 300 g, about 350 g, about 400 g, about 450 g, about 500 g, about 550 g, or about 600 g macadamia oil. In some embodiments, a carrier of about 2050 g comprises from about 100 to about 600 g, from about 200 g to about 500 g, from about 300 g to about 400 g maracuja oil. In some embodiments, a carrier of about 2050 g comprises about 100 g, about 150 g, about 200 g, about 250 g, about 300 g, about 350 g, about 400 g, about 450 g, about 500 g, about 550 g, or about 600 g maracuja oil. In some embodiments, a carrier of about 2050 g comprises about 308 g macadamia oil and about 308 g maracuja oil.

In some embodiments, the carrier comprises about 15%, about 20%, about 25%, about 30%, about 35%, about 40%, about 45%, about 50%, about 55%, or about 60% w/w of one or more emulsifiers. In some embodiments, the carrier comprises two emulsifiers. In some embodiments, the carrier comprises two emulsifiers in a ratio by weight of about 1:2, 1:3, 1:4, 1:5, 1:6, 1:7, 1:9, or 1:10 relative to each other.

In some embodiments, a carrier of about 2050 g comprises from about 300 to about 1500 g, from about 500 g to about 1000 g, from about 700 g to about 800 g one or more emulsifiers. In some embodiments, a carrier of about 2050 g comprises about 300 g, about 400 g, about 500 g, about 600 g, about 700 g, about 800 g, about 900 g, about 1000 g, about 1100 g, about 1200 g, about 1300 g, about 1400 g, about 1500 g one or more emulsifiers. In some embodiments, the emulsifier comprises a nonionic emulsifier. In some embodiments, the emulsifier comprises polysorbate 80. In some embodiments, a carrier of about 2050 g comprises about 742 g polysorbate 80.

In some embodiments, the carrier comprises about 15%, about 20%, about 25%, about 30%, about 35%, about 40%, about 45%, about 50%, about 55%, about 60%, about 65%, about 70% w/w water. In some embodiments, a carrier of about 2050 g comprises from about 300 to about 1500 g, about 500 g to about 1000 g, from about 700 to about 800 g water. In some embodiments, a carrier of about 2050 g comprises about 300 g, about 400 g, about 500 g, about 600 g, about 700 g, about 800 g, about 900 g, about 1000 g, about 1100 g, about 1200 g, about 1300 g, about 1400 g, or about 1500 g water. In some embodiments, the water is distilled water. In some embodiments, the water is deionized water.

In some embodiments, a transdermal delivery formulation described herein comprises a formulation as described Table 5.

TABLE 5

| Transdermal | Volume | Density | Calculated | Percentage |
|---|---|---|---|---|
| Accelerant | added (ml) | (g/ml) | weight (g) | (wt/wt) |
| Citric Acid | 340 | 1.1 | 374 | 12.0% |
| Powder | | | | |
| Apple Cider | 800 | 0.8 | 640 | 20.5% |
| Vinegar | | | | |
| Beetroot Powder | 60 | 0.74 | 44.4 | 1.4% |
| Carrier | | | | |
| Polysorbate 80 | 700 | 1.06 | 742 | 23.8% |
| Distilled Water | 700 | 1 | 700 | 22.5% |
| Macadamia Oil | 350 | 0.88 | 308 | 9.9% |
| Maracuja Oil | 350 | 0.88 | 308 | 9.9% |
| Total | | | 3116.4 | |

### Emulsions

Emulsions are a dispersion of droplets of a first liquid in an immiscible second liquid. In some embodiments, the emulsion provided herein is an oil in water emulsion. In some embodiments, the emulsion provided herein is a water in oil emulsion. In some embodiments, a transdermal accelerant described herein is combined with a carrier described herein.

In some embodiments, emulsions are generated by applying an energy to disperse a first phase of the emulsion in a second phase. In some embodiments, the energy is applied by vortexing, shearing, stirring, shaking, vortexing, shearing, sonicating, homogenizing, blending, tumbling, extruding, milling, grinding, lyophilization, electrolyzing, heating, cooling, atomizing, or any combination thereof. In some embodiments, the emulsion is generated using a paddle mixer, a rotor-stator mixer, a blender, a homogenizer, a sonicator, a vortexer, a high pressure homogenizer, or a microfluidizer.

In some embodiments, emulsions described herein comprise particles from about 10 nm to about 300 nm, from about 20 nm to about 500 nm, from about 500 nm to about 100 μM. In some embodiments, an emulsion described herein comprises a mean particle size of about 10 nm, about 50 nm, about 100 nm, about 200 nm, about 300 nm, about 400 nm, about 500 nm, about 600 nm, about 700 nm, about 800 nm, about 900 nm, about 1000 nm, about 5 μM, about 10 μM, about 15 μM, about 20 in, about 25 μM, about in, about 35 μM, about 40 μM, about 45 in, about 50 μM, about 60 μm, about 70 μm, about 80 μm, about 90 μm, or about 100 μm. A “micro-emulsion,” as provided herein, comprises an emulsion comprising a median particle size from about 10 nm to about 300 nm. A “nano-emulsion” comprises an emulsion comprising a median particle size from about 20 nm to about 500 nm. A “macro-emulsion” comprises an emulsion comprising a median particle size from about 500 nm to about 100 μm.

Emulsion stability is measured by the growth in median particle size of the emulsion over a period of time. In some embodiments, the median particle size is measured over one hour, one day, one week, two weeks, one month, two months, three months, six months, nine months, or one year. In some embodiments, the median particle size increases less than about 10%, about 20%, about 30%, about 40%, about 50%, or about 60% over the period of time.

### Application

In some embodiments, a transdermal delivery formulation described herein is applied directly to a skin surface. In some embodiments, the transdermal delivery formulation is applied to a mucosal surface. In some embodiments, the transdermal delivery formulation is applied as a lotion, a gel, a cream, an ointment, a liniment, a paste, a film, an encapsulation, or a liquid.

Ionization of the skin surface, in some embodiments, increases the conductivity of the skin. In some embodiments, increasing conductivity of the skin improves penetration of a transdermal delivery formulation as described herein. In some embodiments, an ionic solution is applied to the skin after application of the transdermal delivery formulation. In some embodiments, a further overlay of an ionized water is applied following application of the transdermal delivery formulation. In some embodiments, the ionized water is in a liquid form. In some embodiments, the ionized water is in a gaseous form. In some embodiments, the ionized water is applied as a wash, a spray, a steam, or any other form to provide contact with the skin.

### Additional Additives

Transdermal delivery formulations may further comprise one or more active agents. In some embodiments, an active agent comprises a therapeutic agent, a drug, a nutraceutical, a nutrient, and/or other compound. In some embodiments, the therapeutic agent comprises a biologic, a protein, a peptide, a small molecule, a macromolecule, a nucleic acid, another pharmaceutically or physiologically active ingredient, or any combination thereof. In some embodiments, the nutraceutical comprises a compound or food product with an additional pharmaceutical effect.

Transdermal delivery formulations may alternatively or additionally comprise one or more viscosity enhancer, nutrient, plant powder or extract, amino acid, a vitamin, or any combination thereof. Representative viscosity enhancers may be selected from the group consisting of lecithin, aloe vera, glycerin, plant oil, animal oil, and collagen.

Representative nutrients may be selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

Representative plant powders or extracts may be selected from the group consisting of bacopa powder, bamboo extract powder, beet powder, blueberry extract, ginkgo biloba, ginger, grape seed extract, green tea, jojoba, nutmeg, olive leaf, pomegranate, and turmeric.

Representative amino acids may be selected from the group consisting of alanine, arginine, leucine, isoleucine, valine, glutamine, glycine, histidine, leucine, lysine, methionine, proline, serine, threonine, and valine.

Within other embodiments, the presently disclosed transdermal delivery formulations comprise a transdermal accelerant wherein the nitrate source is a plant-based nitrate source, such as an arugula, spinach, leafy green vegetables, or beetroot nitrate source.

Within related aspects, the presently disclosed transdermal delivery formulations comprise a carrier serum that comprises a nitrate source, such as a plant-based nitrate source, such as an arugula, spinach, leafy green vegetables, or beetroot nitrate source.

As used herein, the terms “nitrate” and “NO3-” refer interchangeably to an inorganic precursor of “nitric oxide.” Laue, Ullmann's Encyclopedia of Industrial Chemistry (Ed. Weinheim, Wiley-VCH 2006). Plant sources that are especially high in inorganic nitrate include leafy green vegetables, such as spinach and arugula, and beetroot. Dietary nitrate supplementation has been shown to increase endurance exercise performance.

Within yet other aspects, the transdermal delivery formulation further comprises viscosity enhancer, nutrient, plant powder or extract, amino acid, a vitamin, or any combination thereof. As used herein, the term “viscosity” and “thickness” refers interchangeably to the resistance of a fluid, compound, serum, composition, or formulation to deformation at a given rate. “Viscosity enhancer” refers to compounds that are added to increase the “viscosity” and “thickness” of a composition or formulation. “Viscosity” and “thickness” of a fluid, compound, serum, composition, or formulation typically decreases with increasing temperature. “Viscosity” can be measured with a viscometer, a rheometer, a Zahn cup, or a Ford viscosity cup. For example, oil “viscosity” can be determined using a Cannon-Fenske capillary viscometer, after calibration with 60% sucrose solution in a constant temperature bath regulated to +/−0.05° C. The SI unit of viscosity is the newton-second per square meter (N·s/m2), pascal·second (Pa·s), kilogram per meter per second (kg·m−1·s−1), and Poiseuille (PI). The CGS unit is the poise (P, or g·cm−1s−1=0.1 Pa·s).

Certain core transdermal delivery formulations comprise a viscosity enhancer that is selected from the group consisting of lecithin, aloe vera, glycerin, plant oil, animal oil, and collagen. Other transdermal delivery formulations comprise a nutrient that is selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

Further core transdermal delivery formulations comprise a plant powder, oil, or extract that is selected from the group consisting of bacopa powder, bamboo extract powder, beet powder, blueberry extract, Ginkgo biloba, ginger, grape seed extract, green tea, jojoba, nutmeg, olive leaf, pomegranate, and turmeric. Other core transdermal delivery formulations comprise an amino acid that is selected from the group consisting of alanine, arginine, leucine, isoleucine, valine, glutamine, glycine, histidine, leucine, lysine, methionine, proline, serine, threonine, and valine. Yet other core transdermal delivery formulations comprise a vitamin that is selected from the group consisting of vitamin A, vitamin B-1, vitamin B-2, vitamin B-3, vitamin B-7 & 8 inositol, vitamin B-9 (folic acid), vitamin B-12, vitamin C, vitamin D-3, and vitamin E.

Also provided herein are transdermal delivery formulations that are enriched with one or more components for enhancing nutrition and optimizing health (“Core”), immune support, restful sleep (“Sleep”), exercise performance (“Exercise”) and nitric oxide metabolism (“iNOS”), cognitive function (“Cognition”), or any combination thereof. Those transdermal delivery formulations are exemplified by the representative transdermal delivery formulations presented in Table 6.

Transdermal delivery formulations for enhancing exercise performance may comprise one or more of a hydration electrolyte, cinnamic acid, phenylalanine, resveratrol, and carnitine.

Electrolytes comprise positively and negatively charged ions that conduct electrical activity to perform various functions within the body. Electrolyte concentrations in vivo are regulated to maintain fluid balance, muscle contraction, and neural activity, which are all essential to high levels of exercise performance and basic daily functions. Electrolytes that are lost through sweating include sodium (Na+), and chloride (Cl−), potassium (K+), Magnesium (Mg2+), and Calcium (Ca2+). Peak exercise performance, therefore, requires replenishment of hydration electrolytes. Athletes exercising in extreme conditions (for three or more hours continuously) must consume electrolytes to prevent dehydration and/or hyponatremia.

Arginine a basic amino acid which is a constituent of most proteins. It is an essential nutrient in the diet of vertebrates. Chemical formula: HN═C(NH2)NH(CH2)3CH(NH2)COOH.

Cinnamic acid plays key roles in the formation of complex phenolic compounds, which occur primarily as esters of quinic acid or esterified to malic or tartaric acids, or sugars. Chlorogenic acid (5-caffeoylquinic acid) is an important cinnamic acid that is observed in fruits and contributes 25% of the dry weight of the bilberry (Vaccinium) fruit. Chlorogenic acid can be isolated from green coffee beans, and forms a black compound with iron, believed to be responsible for the blackening of cut or cooked potatoes. Anthocyanin and flavonoid glycosides are also acylated by cinnamic acids through sugar hydroxyl groups, with p-coumaric acid the most common acylating agent. In addition to forming esters, hydroxylated cinnamic acids also form glycosides with sugars.

Phenylalanine stimulates plasma cholecystokinin (CCK) and pyloric pressures, both of which are important in the regulation of energy intake and gastric emptying. Gastric emptying is a key determinant of postprandial blood glucose.

Resveratrol is a plant polyphenol micronutrient found in peanuts, blueberries, red grapes, and wine. Resveratrol has antioxidant, anti-inflammatory, immunomodulatory, glucose and lipid regulatory, neuroprotective, and cardiovascular protective effects, therefore, can protect against diverse chronic diseases, such as cardiovascular diseases (CVDs), cancer, liver diseases, obesity, diabetes, Alzheimer's disease.

Coenzyme Q10 (“CoQ10”) is a ubiquinone, which is an essential component of the energy-generating machinery within the mitochondria. The safety and efficacy of CoQ10 in prolonging exercise performance has been reported

CoQ10 participates in redox reactions within the electron transport chain at the mitochondrial level facilitating the production of adenosine triphosphate (ATP) to a mobile redox agent shuttling electrons and protons in the electron transport chain. CoQ10 also has a lipophilic antioxidant effect that protects the DNA, the phospholipids and the mitochondrial membrane proteins against the lipid peroxidation. CoQ10 acts as an antioxidant in both the mitochondria and lipid membranes by scavenging reactive oxygen species (ROS), either directly or in conjunction with a-tocopherol (vitamin E).

L-carnitine (“carnitine”) is used by highly competitive and trained athletes to affect physical performance. The primary function of carnitine is the transport of long-chain fatty acids into the mitochondrial matrix for their conversion in energy, via β-oxidation process. Carnitine reacts with acetyl-CoA and maintains the acetyl-CoA/CoA ratio in the cell to, thereby, regulate pyruvate dehydrogenase activity. Carnitine also plays an important role in the regulation of metabolic pathways involved in skeletal muscle protein balance: proteolysis and protein synthesis.

Transdermal delivery formulations for enhancing restful sleep may comprise one or more of gamma-aminobutyric acid (GABA), melatonin, and tryptophan.

GABA is the main inhibitory neurotransmitter of the CNS. It is well established that activation of GABAA receptors favors sleep. GABA is a non-proteinogenic amino acid and is the main inhibitory neurotransmitter in the mammalian brain. GABA's stress-reducing and sleep enhancing effects are well established.

Melatonin is a hormone (an “indoleamine”) that the brain produces in response to darkness.

Melatonin affects synchronizing of circadian rhythms, timing of sleep-wake cycling, and regulation of blood pressure. Many of its effects are through activation of the melatonin receptors, while others are due to its role as an antioxidant. Mitochondria are the main cell organelles that produce the antioxidant melatonin, which indicates that melatonin is an ancient molecule that primarily provided the earliest cells protection from the destructive actions of oxygen. Melatonin is commonly used as a dietary supplement and medication in the treatment of sleep disorders such as insomnia and circadian rhythm sleep disorders.

Tryptophan is an amino acid that produces an increase in rated subjective sleepiness and a decrease in sleep latency (time to sleep). Tryptophan may also have additional effects such as decrease in total wakefulness and/or increase in sleep time. Tryptophan is more effective in subjects having mild insomnia or in normal subjects reporting a longer-than-average sleep latency.

Transdermal delivery formulations for enhancing nitric oxide metabolism may comprise one or more of vitamin D, vitamin C, citrulline, ribose ATP blood carrier, beetroot, curcumin (turmeric), fish oil, and threonine.

Nitric oxide is a small molecule gas that is produced in vivo by epithelial cells (e.g., the inner lining of blood vessels, the blood-brain barrier, and barriers in the gut and around reproductive structures). Nitric oxide is a universal messenger, which is made ubiquitously and is involved in detoxification and the urea cycle, tissue regeneration, blood flow, prevention of atherosclerosis, and regulation of inflammation and oxidation. Maintaining a large reservoir of nitric oxide through its in vivo intake or via the uptake of nitric oxide precursors (e.g., arginine, citrulline, and carnitine) is important to health and wellness.

Beetroot possesses high nutritional value and is a primary dietary source of nitrate. The effects of beetroot intake on cardiovascular health with respect to nitric oxide production and blood pressure are well established.

Citrulline is an α-amino acid that is a key intermediate in the urea cycle, the pathway by which mammals excrete ammonia by converting it into urea. Citrulline is also produced as a byproduct of the enzymatic production of nitric oxide from the amino acid arginine, catalyzed by nitric oxide synthase.

Curcumin (turmeric) is a naturally occurring, dietary polyphenolic phytochemical having anti-inflammatory properties. Curcumin (as well as demethoxycurcumin, bisdemethoxycurcumin, and diacetylcurcumin) inhibits activation of free radical-activated transcription factors, such as nuclear factor κB and AP-1, and reduces production of pro-inflammatory cytokines such as tumor necrosis factor-α, interleukin-1β, and interleukin-8. Inducible nitric oxide synthase (iNOS) is an inflammation-induced enzyme that catalyzes the production of nitric oxide.

Vitamin D is a group of lipid-soluble secosteroids that are responsible for increasing intestinal absorption of Ca2+, Mg2+, and PO₄³⁻. Vitamin D encompasses cholecalciferol (vitamin D₃), which is synthesized in the lower layers of skin epidermis through a photo-chemical reaction of UVB light from sun exposure and is obtained dietarily in fish oil. The active vitamin D metabolite calcitriol mediates its biological effects by binding to the vitamin D receptor (VDR) located in the nuclei of target cells. The binding of calcitriol to the VDR allows the VDR to act as a transcription factor that modulates the gene expression of transport proteins (such as Transient Receptor Potential Cation Channel Subfamily V Member 6 (TRPV6) and calbindin), which are involved in calcium absorption in the intestine. VDR regulates cell proliferation and differentiation and affects the synthesis of neurotrophic factors, nitric oxide synthase, and glutathione.

Vitamin C (ascorbic acid) is a water-soluble vitamin found in citrus and other fruits and vegetables. Vitamin C improves endothelium-dependent vasodilation by restoring nitric oxide activity in essential hypertension. Vitamin C also restores the nitric oxide synthase inhibitory effects of N-methylarginine.

Transdermal delivery formulations for enhancing cognitive performance may comprise one or more of silicon, leucine, lysine, isoleucine, valine, threonine, phenylalanine, methionine, histidine, tryptophan, vitamin B-5 (pantothenic acid), vitamin B-7 (biotin), vitamin K, acetyl L carnitine, palmitoyl-ethanolamide (PEA), taurine, periwinkle, artichoke, bacopa, ginkgo biloba, nutmeg, alanine, and tyrosine.

The B vitamins comprise a group of eight water soluble vitamins that perform essential, closely inter-related roles in cellular functioning. In particular, the collective effects are of B vitamins are critical to numerous aspects of brain function, including energy production, DNA and RNA synthesis and repair, genomic and non-genomic methylation, and the synthesis of numerous neurochemicals and signaling molecules.

Pantothenic acid (vitamin B5) is a substrate for the synthesis of the ubiquitous coenzyme A (CoA). Beyond its role in oxidative metabolism, CoA contributes to the structure and function of brain cells via its involvement in the synthesis of cholesterol, amino acids, phospholipids, and fatty acids. Pantothenic acid is also involved in the synthesis of multiple neurotransmitters and steroid hormones via pathways involving CoA.

Vitamin B-7 (biotin), vitamin B-12, and thiamine, play unique, intersecting, essential roles in mitochondrial metabolism of glucose, fatty acids, and amino acids, respectively, thereby contributing substrates to the citric acid cycle.

Vitamin K is a fat-soluble nutrient mainly found in green leafy vegetables as phylloquinone (Vitamin K1). This vitamin is widely known for its procoagulant effect. It acts as a cofactor for the enzyme that allows the activation of vitamin K-dependent factors (II, VII, IX, X, protein C, and protein S). Vitamin K is involved in the metabolism of the central nervous system (CNS), suggesting the possibility that a vitamin K deficiency might be related to the onset of cognitive impairment.

Moreover, vitamin K is involved in the metabolism of sphingolipids, which participate in the proliferation, differentiation, and survival of brain cells. An altered expression in sphingolipid profile is associated with neuroinflammation and neurodegeneration. Evidence of a direct correlation between vitamin K levels and cognitive performance (including visual memory, verbal fluency, and brain volume) have been reported.

Palmitoyl-ethanolamide (PEA) has been associated with neuroprotective and anti-inflammatory properties. Studies conducted in animal models of neurodegeneration demonstrate that PEA improves neurobehavioral functions, including memory and learning, by reducing oxidative stress and pro-inflammatory and astrocyte marker expression as well as rebalancing glutamatergic transmission. PEA promotes neurogenesis, especially in the hippocampus, neuronal viability and survival, and microtubule associated protein and brain derived neurotrophic factor expression, while inhibiting mast cell infiltration/degranulation and astrocyte activation.

Taurine is a key functional amino acid with many functions in the nervous system. The effects of taurine on cognitive function have aroused increasing attention. First, the fluctuations of taurine and its transporters are associated with cognitive impairments in physiology and pathology. Taurine supplements in cognitive impairment of different physiologies, pathologies, and toxicologies have been demonstrated to significantly improve and restore cognition.

TABLE 6
Representative Components within Distinct Transdermal Delivery Formulations

| | | TRANSDERMAL DELIVERY FORMULATION | | | | |
|---|---|---|---|---|---|---|
| CATEGORY | COMPONENT | CORE | EXERCISE | SLEEP | iNOS | COGNITION |
| Energy | Hydration | −− | ++++ | −− | −− | −− |
| Enhancement | Electrolytes | −− | ++++ | −− | −− | −− |
| & | Olive | −− | ++++ | −− | −− | −− |
| Exercise | Cinnamic Acid | −− | ++++ | −− | −− | −− |
| Performance | (Cinnamon) | −− | ++++ | −− | −− | −− |
| | Phenylalanine | −− | ++++ | −− | −− | −− |
| | CoQ10 | −− | ++++ | −− | −− | −− |
| | Resveratrol | ++ | ++++ | ++ | ++ | ++ |
| | Carnitine | ++ | ++++ | ++ | ++ | ++ |
| | Bamboo | ++ | ++++ | ++ | ++ | ++ |
| Sleep | Gamma- | −− | −− | ++++ | −− | −− |
| Enhancement | Aminobutyric | | | | | |
| | Acid (GABA) | | | | | |
| | Melatonin | −− | −− | ++++ | −− | −− |
| | Tryptophan | ++ | ++ | ++++ | ++ | ++ |
| iNOS | Vitamin D | −− | −− | −− | ++++ | −− |
| Enhancement | Citrulline | ++ | ++ | ++ | ++++ | ++ |
| | Ribose ATP | ++ | ++ | ++ | ++++ | ++ |
| | Blood Carrier | | | | | |
| | Beetroot | ++ | ++ | ++ | ++++ | ++ |
| | Curcumin | ++ | ++ | ++ | ++++ | ++ |
| | (Turmeric) | | | | | |
| | Fish Oil | ++ | ++ | ++ | ++++ | ++ |
| | Threonine | ++ | ++ | ++ | ++++ | ++ |
| | Vitamin C | ++ | ++ | ++ | ++++ | ++ |
| | (Ascorbic Acid) | | | | | |
| Cognitive | silicon | −− | −− | −− | −− | ++++ |
| Enhancement | Amino acids | −− | −− | −− | −− | ++++ |
| | Vitamin B-5 | −− | −− | −− | −− | ++++ |
| | (Pantothenic | | | | | |
| | Acid) | | | | | |
| | Vitamin B-7 | −− | −− | −− | −− | ++++ |
| | (Biotin) | | | | | |
| | Vitamin K | −− | −− | −− | −− | ++++ |
| | Acetyl L | ++ | ++ | ++ | ++ | ++++ |
| | Carnitine | | | | | |
| | Palmitoyl- | ++ | ++ | ++ | ++ | ++++ |
| | ethanolamide | | | | | |
| | (PEA) | | | | | |
| | Taurine | ++ | ++ | ++ | ++ | ++++ |
| | Periwinkle | ++ | ++ | ++ | ++ | ++++ |
| | Artichoke | ++ | ++ | ++ | ++ | ++++ |
| | Bacopa | ++ | ++ | ++ | ++ | ++++ |
| | Ginkgo Biloba | ++ | ++ | ++ | ++ | ++++ |
| | Nutmeg | ++ | ++ | ++ | ++ | ++++ |
| | Alanine | ++ | ++ | ++ | ++ | + |
| | Tyrosine | ++ | ++ | ++ | ++ | ++++ |
| | Vitamin C Serum | ++ | ++ | ++ | ++ | ++++ |

−− absent from a Transdermal Delivery Formulation
++ present in a Transdermal Delivery Formulation
++++ unique to (or at an elevated concentration relative to “core”) in a Transdermal Delivery Formulation

In some embodiments, a transdermal delivery formulation described herein further comprises one or more additional ingredients comprising acetyl-l-carnitine (alc), alanine, alpha lipoic acid, arginine, artichoke extract, ashwagandha, bacopa powder, bamboo extract powder, basil powder, beet powder, calcium carbonate, blueberry extract, aloe vera, choline bitartrate, collagen protein peptides, collagen powder, citrulline malate, curcumin powder, creatine, CoQ10, kale powder, folic acid, gingko biloba, glutamine, glycerin, ginger, glycine, grape seed extract, green tea, inositol (vitamin B8), histidine, jojoba, pomegranate powder, magnesium glycinate, leucine, lysine, lecithin (sun flower), niacinamide (vitamin B3), nutmeg powder, niacin, methionine, NALT (acetyl tyrosine), NAC, lutein, olive leaf, pea powder, periwinkle, phenylalanine, potassium, proline, ribose, DAA (d-aspartic acid), serine, hyaluronic acid, taurine, threonine, tryptophan, turmeric, theanine, valine (amino acid), valerian root powder, zinc oxide, vitamin A, vitamin B complex, vitamin B-1, vitamin B-2, vitamin B-5, vitamin B-6 (pyridoxine), vitamin B-7&8, vitamin B-9 (folic acid), vitamin B-12, vitamin C (ascorbic acid), vitamin D-3 (cholecalciferol), vitamin E, vitamin K-2, ginseng, isoleucine, almond oil, broccoli seed oil, collagen liquid, avocado oil, chamomile liquid, vitamin E oil, glycerin (conditioning), grapefruit seed, grape seed oil, gotu kola oil, kava liquid, virgin algae oil (fish oil), jojoba extract oil (organic), lavender oil, macadamia nut oil, meadowfoam seed oil, peppermint oil, maracuja oil (passionfruit), primrose oil, pomegranate oil, BCAA, GABA, collagen oil, virgin algae oil, ashwagandha powder, collagen peptides, guarana powder, and huperzine. In some embodiments, the transdermal delivery formulation comprises more than one, more than 2, more than 3, more than 4, more than 5, more than 6, more than 7, more than 8, more than 9, more than 10, more than 11, more than 12, more than 13, more than 14, more than 15, more than 16, more than 17, more than 18, more than 19, more than 20 supplemental ingredients.

Other embodiments on the present disclosure provides methods for the manufacture of transdermal delivery formulations as disclosed herein for the epicutaneous administrations of drugs, nutrients, and/or other compounds to a human subject or domestic, veterinary, or agricultural animal.

### EXEMPLARY EMBODIMENTS

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

In one aspect, provided herein is a transdermal delivery formulation, comprising at least one weak organic acid, at least one active agent, water, one or more oils comprising at least one unsaturated long-chain fatty acid selected from oleic acid and linoleic acid, and polysorbate 80. In some embodiments, the at least one weak organic acid is present in total in an amount from 2.0% to 45.0% w/w of the formulation. In some embodiments, water is present in a weight percentage in the range of 15% to 70% of the formulation. In some embodiments, the one or more oils in total have a weight percentage in the range of 5% to 35% of the formulation. In some embodiments, the polysorbate 80 is present in a weight percentage in the range of 15% to 40% of the formulation. In some embodiments, the transdermal delivery formulation has a pH in the range of 2.0 to 6.0. In some embodiments, the polysorbate 80, the one or more oils in total, and the water comprise a weight ratio in the range of X:Y:Z, wherein X=1-5, Y=1-6, and Z=1-6. In some embodiments, the transdermal delivery formulation is capable of delivering the at least one agent to the bloodstream of a subject when applied to the skin of the subject.

In some embodiments of any of the aspects and embodiments herein that include a transdermal delivery formulation, the at least one active agent comprises arginine, citrulline, citrulline malate, beetroot, creatine, glutamine, leucine, norvaline, ornithine, histidine, beta-alanine, phenylalanine, agmatine, betaine, L-theanine, glutathione, or any nitrate, nitrite, nitro, or nitroso compound, or a salt thereof, or any combination thereof.

In some embodiments of any of the aspects and embodiments herein that include a transdermal delivery formulation, the at least one active agent further comprises ascorbic acid or a derivative thereof.

In some embodiments of any of the aspects and embodiments herein that include a transdermal delivery formulation, the at least one weak organic acid is present in total in an amount from 4.5% to 45.0% w/w.

In some embodiments of any of the aspects and embodiments herein that include a transdermal delivery formulation, the at least one weak organic acid is present in total in an amount from 10.0% to 45.0% w/w.

In some embodiments of any of the aspects and embodiments herein that include a transdermal delivery formulation, the formulation comprises about 20%, 25%, 30%, 35%, 40%, 45%, 50%, 55%, 60%, 65%, 70%, 75%, or 80% (w/w) of the at least one weak organic acid.

In some embodiments of any of the aspects and embodiments herein that include a transdermal delivery formulation, the formulation comprises about 60%, 65%, 70%, 75%, or 80% (w/w) of the total of the polysorbate 80, the one or more oils in total, and water.

In some embodiments of any of the aspects and embodiments herein that include a transdermal delivery formulation, the at least one weak organic acid comprises two weak organic acids at a ratio by weight of about 1:1, 1:2, 1:3, 1:4, 1:5, 1:6, 1:7, 1:8, 1:9, 1:10, 1:100, or 1:1000 relative to each other.

In some embodiments of any of the aspects and embodiments herein that include a transdermal delivery formulation, the at least one weak organic acid comprises about 80%, 85%, 90%, 95%, 97%, or 99% (w/w) of one or more weak organic acids.

In some embodiments of any of the aspects and embodiments herein that include a transdermal delivery formulation, the at least one weak organic acid has a median pKa of from 3.0 to 5.5.

In some embodiments of any of the aspects and embodiments herein that include a transdermal delivery formulation, the at least one weak organic acid has a median pKa of from 4.0 to 5.0.

In some embodiments of any of the aspects and embodiments herein that include a transdermal delivery formulation, the at least one weak organic acid has a median pKa of about 4.6.

In some embodiments of any of the aspects and embodiments herein that include a transdermal delivery formulation, the at least one weak organic acid comprises a mono-, di-, or tri-carboxylic acid of chain length (R) between 1 and 16.

In some embodiments of any of the aspects and embodiments herein that include a transdermal delivery formulation, the at least one weak organic acid comprises a mono- or poly-hydroxy moiety of 0-14.

In some embodiments of any of the aspects and embodiments herein that include a transdermal delivery formulation, the at least one weak organic acid comprises lactic acid, acetic acid, formic acid, citric acid, oxalic acid, gallic acid, malic acid, maleic acid, malonic acid, succinic acid, tartaric acid, fumaric acid, or any combination thereof.

In some embodiments of any of the aspects and embodiments herein that include a transdermal delivery formulation, the weak organic acid is citric acid or acetic acid.

In some embodiments of any of the aspects and embodiments herein that include a transdermal delivery formulation, the at least one weak organic acid comprises citric acid and acetic acid.

In some embodiments of any of the aspects and embodiments herein that include a transdermal delivery formulation, the acetic acid is apple cider vinegar.

In some embodiments of any of the aspects and embodiments herein that include a transdermal delivery formulation, the formulation comprises two oils comprising an unsaturated long-chain fatty acid, wherein the two oils are in a ratio by weight of about 1:1, 1:2, 1:3, 1:4, 1:5, 1:6, 1:7, 1:8, 1:9, 1:10, 1:100, or 1:1000 relative to each other.

In some embodiments of any of the aspects and embodiments herein that include a transdermal delivery formulation, the at least one unsaturated long-chain fatty acid comprises a chain of from 12 to 26 carbons.

In some embodiments of any of the aspects and embodiments herein that include a transdermal delivery formulation, the at least one unsaturated long-chain fatty acid comprises one or more double bonds in a cis configuration.

In some embodiments of any of the aspects and embodiments herein that include a transdermal delivery formulation, the at least one unsaturated long-chain fatty acid comprises adrenic acid, arachidonic acid, arachidic acid, behenic acid, brassidic acid, cervonic acid, cis-vaccenic acid, dihomo-γ-linolenic acid, docosadienoic acid, eicosadienoic acid, eicosapentaenoic acid, eicosatetraenoic acid, eicosenoic acid, elaidic acid, erucic acid, gadoleic acid, gondoic acid, herring acid, lauric acid, lignoceric acid, linoleic acid, linolelaidic acid, margaric acid, margoleic acid, mead acid, myristoleic acid, nervonic acid, oleic acid, ozubondo acid, palmitic acid, palmitoleic acid, trans-palmitoleic acid, paullinic acid, petroselinic acid, pinolenic acid, sapienic acid, sardine acid, stearic acid, stearidonic acid, tetracosapentaenoic acid, α-linolenic acid, γ-linolenic acid, or any combination thereof.

In some embodiments of any of the aspects and embodiments herein that include a transdermal delivery formulation, the at least one unsaturated long-chain fatty acid is oleic acid or linoleic acid.

In some embodiments of any of the aspects and embodiments herein that include a transdermal delivery formulation, the at least one long-chain fatty acid comprises oleic acid and linoleic acid.

In some embodiments of any of the aspects and embodiments herein that include a transdermal delivery formulation, the one or more oils comprising an unsaturated long-chain fatty acid is one or more plant or animal oils.

In some embodiments of any of the aspects and embodiments herein that include a transdermal delivery formulation, the one or more plant oils is selected from the group consisting of vegetable oil, nut oil, and seed oil.

In some embodiments of any of the aspects and embodiments herein that include a transdermal delivery formulation, the one or more plant or animal oils comprises macadamia oil, maracuja (passion fruit) oil, safflower oil, sunflower oil, olive oil, avocado oil, canola oil, coconut oil, corn oil, cottonseed oil, flaxseed or linseed oil, grape seed oil, hemp seed oil, palm oil, peanut oil, rice bran oil, sesame oil, soybean oil, brazil nut oil, almond oil, walnut oil, pecan oil, jojoba oil, chia seed oil, wallflower seed oil, mustard oil, borage oil, black currant oil, evening primrose oil, chicken fat, cartilage oil, cod liver oil, herring oil, mackerel oil, salmon oil, menhaden oil, sardine oil, or any combination thereof.

In some embodiments of any of the aspects and embodiments herein that include a transdermal delivery formulation, the one or more plant or animal oils is macadamia oil or maracuja (passion fruit) oil, or a combination thereof.

In some embodiments of any of the aspects and embodiments herein that include a transdermal delivery formulation, the one or more plant or animal oils comprises macadamia oil and maracuja (passion fruit) oil.

In some embodiments of any of the aspects and embodiments herein that include a transdermal delivery formulation, the formulation comprises a lotion, gel, cream, ointment, liniment, paste, film, encapsulation, or liquid.

In some embodiments of any of the aspects and embodiments herein that include a transdermal delivery formulation, the formulation comprises a nitrate source.

In some embodiments of any of the aspects and embodiments herein that include a transdermal delivery formulation, the nitrate source is a plant-based nitrate source.

In some embodiments of any of the aspects and embodiments herein that include a transdermal delivery formulation, the plant-based nitrate source comprises arugula, spinach, leafy green vegetables, beetroot, or any combination thereof.

In some embodiments of any of the aspects and embodiments herein that include a transdermal delivery formulation, the plant-based nitrate source is beetroot, and the beetroot is a dried beetroot powder.

In some embodiments of any of the aspects and embodiments herein that include a transdermal delivery formulation, the formulation comprises about 2.0%, 2.5%, 3.0%, 3.5%, 4.0%, 4.5%, 5.0%, 5.5%, 6.0%, 6.5%, 7.0%, 7.5%, or 8.0% (w/w) dried beetroot powder.

In some embodiments of any of the aspects and embodiments herein that include a transdermal delivery formulation, the formulation further comprises a viscosity enhancer, a nutrient, a plant powder or extract, an amino acid, a vitamin, or any combination thereof.

In some embodiments of any of the aspects and embodiments herein that include a transdermal delivery formulation, the formulation comprises a viscosity enhancer selected from the group consisting of lecithin, aloe vera, glycerin, a plant oil, an animal oil, and collagen.

In some embodiments of any of the aspects and embodiments herein that include a transdermal delivery formulation, the formulation comprises a nutrient selected from the group consisting of acetyl-L-carnitine, alpha lipoic acid, arginine, potassium, N-acetyl-L-tyrosine (NALT), N-acetylcysteine (NAC), palmitoyl-ethanolamide (PEA), resveratrol, taurine, palmitate, calcium carbonate, choline bitartrate (B-4), creatine, citrulline malate, magnesium glycinate, carnitine, coenzyme Q10 (CoQ10), humic acid, hyaluronic acid, magnesium, selenium, and zinc oxide.

In some embodiments of any of the aspects and embodiments herein that include a transdermal delivery formulation, the formulation comprises a plant powder or extract selected from the group consisting of bacopa powder, bamboo extract powder, beet powder, blueberry extract, ginkgo biloba, ginger, grape seed extract, green tea, jojoba, nutmeg, olive leaf, pomegranate, and turmeric.

In some embodiments of any of the aspects and embodiments herein that include a transdermal delivery formulation, the formulation comprises an amino acid selected from the group consisting of alanine, arginine, leucine, isoleucine, valine, glutamine, glycine, histidine, lysine, methionine, proline, serine, threonine, and valine.

In some embodiments of any of the aspects and embodiments herein that include a transdermal delivery formulation, the formulation comprises a vitamin selected from the group consisting of vitamin A, vitamin B-1, vitamin B-2, vitamin B-3, vitamin B-7, vitamin B-8 (inositol), vitamin B-9 (folic acid), vitamin B-12, vitamin C, vitamin D-3, and vitamin E.

In some embodiments of any of the aspects and embodiments herein that include a transdermal delivery formulation, the transdermal formulation has a pH of from 3.0 to 5.0.

In some embodiments of any of the aspects and embodiments herein that include a transdermal delivery formulation, the transdermal formulation has a pH of from 3.5 to 4.5.

In some embodiments of any of the aspects and embodiments herein that include a transdermal delivery formulation, the transdermal formulation has a viscosity at 20° C. of from 500 to 10,000 centipoise (cP), or from 1,000 to 5,000 cP, or from 1,500 to 4,000 cP, or from 2,000 to 3,000 cP.

In some embodiments of any of the aspects and embodiments herein that include a transdermal delivery formulation, the transdermal delivery formulation forms an emulsion comprising a population of droplets from about 10 nm to about 300 nm in diameter.

In some embodiments of any of the aspects and embodiments herein that include a transdermal delivery formulation, the formulation further comprises one or more components for enhancing nutrition and optimizing health, immune support, restful sleep, exercise performance and nitric oxide metabolism, cognitive function, or any combination thereof.

In some embodiments of any of the aspects and embodiments herein that include a transdermal delivery formulation, the formulation further comprises one or more of a hydration electrolyte, cinnamic acid, phenylalanine, resveratrol, and carnitine.

In some embodiments of any of the aspects and embodiments herein that include a transdermal delivery formulation, the formulation further comprises one or more of gamma-aminobutyric acid (GABA), melatonin, and tryptophan.

In some embodiments of any of the aspects and embodiments herein that include a transdermal delivery formulation, the formulation further comprises one or more of vitamin D, vitamin C, citrulline, ribose ATP blood carrier, beetroot, curcumin (turmeric), fish oil, and threonine.

In some embodiments of any of the aspects and embodiments herein that include a transdermal delivery formulation, the formulation further comprises one or more of silicon, leucine, lysine, isoleucine, valine, threonine, phenylalanine, methionine, histidine, tryptophan, vitamin B-5 (pantothenic acid), vitamin B-7 (biotin), vitamin K, acetyl-L-carnitine, palmitoyl-ethanolamide (PEA), taurine, periwinkle, artichoke, bacopa, ginkgo biloba, nutmeg, alanine, and tyrosine.

In some embodiments of any of the aspects and embodiments herein that include a transdermal delivery formulation, the formulation further comprises one or more additional ingredients, wherein the additional ingredients comprise acetyl-L-carnitine (ALC), alanine, alpha lipoic acid, arginine, artichoke extract, ashwagandha, bacopa powder, bamboo extract powder, basil powder, beet powder, calcium carbonate, blueberry extract, aloe vera, choline bitartrate, collagen protein peptides, collagen powder, citrulline malate, curcumin powder, creatine, coenzyme Q10 (CoQ10), kale powder, folic acid, ginkgo biloba, glutamine, glycerin, ginger, glycine, grape seed extract, green tea, inositol (vitamins B7/B8), histidine, jojoba, pomegranate powder, magnesium glycinate, leucine, lysine, lecithin (sunflower), niacinamide (vitamin B3), nutmeg powder, niacin, methionine, N-acetyl-L-tyrosine (NALT), N-acetylcysteine (NAC), lutein, olive leaf, pea powder, periwinkle, phenylalanine, potassium, proline, ribose, D-aspartic acid (DAA), serine, hyaluronic acid, taurine, threonine, tryptophan, turmeric, theanine, valine, valerian root powder, zinc oxide, vitamin A, vitamin B complex, vitamin B-1, vitamin B-2, vitamin B-5, vitamin B-6 (pyridoxine), vitamin B-7 & B-8, vitamin B-9 (folic acid), vitamin B-12, vitamin C (ascorbic acid), vitamin D-3 (cholecalciferol), vitamin E, vitamin K-2, ginseng, isoleucine, almond oil, broccoli seed oil, collagen liquid, avocado oil, chamomile liquid, vitamin E oil, glycerin (conditioning), grapefruit seed, grape seed oil, gotu kola oil, kava liquid, virgin algae oil (fish oil), jojoba extract oil (organic), lavender oil, macadamia nut oil, meadowfoam seed oil, peppermint oil, maracuja oil (passionfruit), primrose oil, pomegranate oil, branched-chain amino acids (BCAA), gamma-aminobutyric acid (GABA), collagen oil, virgin algae oil, ashwagandha powder, collagen peptides, guarana powder, and huperzine.

In some embodiments of any of the aspects and embodiments herein that include a transdermal delivery formulation, the transdermal delivery formulation comprises more than one, more than 2, more than 3, more than 4, more than 5, more than 6, more than 7, more than 8, more than 9, more than 10, more than 11, more than 12, more than 13, more than 14, more than 15, more than 16, more than 17, more than 18, more than 19, or more than 20 of the additional ingredients.

In some embodiments of any of the aspects and embodiments herein that include a transdermal delivery formulation, the one or more active agents comprise a therapeutic agent, a nutraceutical, or a combination thereof.

In some embodiments of any of the aspects and embodiments herein that include a transdermal delivery formulation, the therapeutic agent comprises a biologic, protein, peptide, small molecule, macromolecule, nucleic acid, or another pharmaceutically or physiologically active ingredient, or any combination thereof.

In another aspect, provided herein is a method of transdermal delivery to a subject, comprising epicutaneous application of the transdermal delivery formulation as described in any of the aspects and embodiments herein to the subject.

In some embodiments, the method further comprises overlaying ionized water onto the epicutaneously applied transdermal delivery formulation.

In another aspect, provided herein is a method of treatment of a disease or condition in a subject, comprising epicutaneous application of the transdermal delivery formulation as described in any of the aspects and embodiments herein and one or more active agents to the subject.

In some embodiments, the one or more active agents comprise a therapeutic agent, a nutraceutical, or a combination thereof.

In some embodiments, the therapeutic agent comprises a biologic, protein, peptide, small molecule, macromolecule, nucleic acid, or another pharmaceutically or physiologically active ingredient, or any combination thereof.

In another aspect, provided herein is a method of enhancing a feature or condition in a subject, comprising epicutaneous application of the transdermal delivery formulation as described in any of the aspects and embodiments herein to the subject.

In some embodiments, the feature is a cosmetic feature.

In some embodiments, the condition is nutrition, general health, quality of sleep, exercise performance, nitric oxide metabolism, cognitive function, or any combination thereof.

In another aspect, provided herein is a method of systemic delivery of an active agent to a subject, comprising epicutaneous application of the transdermal delivery formulation as described in any of the aspects and embodiments herein and one or more active agents to the subject.

In some embodiments, the method further comprises overlaying ionized water onto the epicutaneously applied transdermal delivery formulation.

In some embodiments, the one or more active agents comprise a therapeutic agent, a nutraceutical, or a combination thereof.

In some embodiments, the therapeutic agent comprises a biologic, protein, peptide, small molecule, macromolecule, nucleic acid, or another pharmaceutically or physiologically active ingredient, or any combination thereof.

1. A transdermal delivery formulation comprising: a homogenous mixture of (a) a transdermal accelerant comprising a weak organic acid and (b) a microemulsion comprising a nonionic emulsifier, water, and a cis-unsaturated long-chain fatty acid.

2. The transdermal delivery formulation of embodiment 1 wherein said weak organic acid has a median pKa greater than 2.0.

3. The transdermal delivery formulation of any of embodiments 1-2 wherein said weak organic acid has a median pKa of from 3.0 to 5.5.

4. The transdermal delivery formulation of any of embodiments 1-3 wherein said weak organic acid has a median pKa of from 4.0 to 5.0.

5. The transdermal delivery formulation of any of embodiments 1-4 wherein said weak organic acid has a median pKa of about 4.7.

6. The transdermal delivery formulation of any of embodiments 1-5 wherein said weak organic acid is selected from the group consisting of mono, di or tri carbonic acids of chain lengths (R) between 1-16, optionally comprising a mono or poly hydroxy moieties of 0-14. In some embodiments, the organic acid derives from a linear, branched, or cyclic structure. In some embodiments, the structure is saturated or unsaturated. In some embodiments, the weak organic acid comprises lactic acid, acetic acid, formic acid, citric acid, oxalic acid, g acid, malic acid, maleic acid, tartaric acid, malonic acid, succinic acid, and fumaric acid.

7. The transdermal delivery formulation of any of embodiments 1-6 wherein said weak organic acid is citric acid or acetic acid.

8. The transdermal delivery formulation of any of embodiments 1-7 wherein said transdermal accelerant comprises citric acid and acetic acid.

9. The transdermal delivery formulation of any of embodiments 1-8 wherein said nonionic emulsifier is selected from the group consisting of lecithin, carboxylmethylcellulose, a sorbitan ester, and a polysorbate.

10. The transdermal delivery formulation of any of embodiments 1-9 wherein said nonionic emulsifier is a sorbitan ester selected from the group consisting of sorbitan monolaurate, sorbitan monostearate, sorbitan tristearate, and sorbitan monooleate.

11. The transdermal delivery formulation of any of embodiments 1-10 wherein said nonionic emulsifier is a polysorbate selected from the group consisting of polyoxyethylene (20) sorbitan monolaurate (Polysorbate 20), polyoxyethylene (20) sorbitan monopalmitate (Polysorbate 40), polyoxyethylene (20) sorbitan monostearate (Polysorbate 60), and polyoxyethylene (20) sorbitan monooleate (Polysorbate 80).

12. The transdermal delivery formulation of any of embodiments 1-11 wherein said polysorbate is Polysorbate 80.

13. The transdermal delivery formulation of any of embodiments 1-12 wherein said water is distilled water.

14. The transdermal delivery formulation of any of embodiments 1-13 wherein said cis-unsaturated long-chain fatty acid comprises a chain of from 16 to 26 carbons.

15. The transdermal delivery formulation of any of embodiments 1-14 wherein said 16 to 26 carbon cis-unsaturated long-chain fatty acid comprises one or more double bond at position 4-9, or at position 5-8, or at position 6-7.

16. The transdermal delivery formulation of any of embodiments 1-15 wherein said 16 to 26 carbon unsaturated long-chain fatty acid is selected from the group consisting of Sapienic Acid, Palmitoleic Acid, Margoleic acid, Cis-Vaccenic Acid, Oleic Acid, Petroselinic Acid, Linoleic Acid, Eicosenoic Acid, Gadoleic Acid, Eicosadienoic acid, Erucic acid, Docosadienoic Acid, and Nervonic acid.

17. The transdermal delivery formulation of any of embodiments 1-16 wherein said 16 to 26 carbon unsaturated long-chain fatty acid is Oleic Acid or Linoleic Acid.

18. The transdermal delivery formulation of any of embodiments 1-17 wherein said 16 to 26 carbon unsaturated long-chain fatty acid comprises Oleic Acid and Linoleic Acid.

19. The transdermal delivery formulation of any of embodiments 1-18 wherein said unsaturated long-chain fatty acid is comprised within a plant oil. 20. The transdermal delivery formulation of any of embodiments 1-19 wherein said plant oil is selected from the group consisting of vegetable oil, nut oil and seed oil.

20. The transdermal delivery formulation of any of embodiments 1-19 wherein said plant oil is selected from the group consisting of Macadamia Oil, Maracuja (Passion Fruit) Oil, Safflower Oil, Sunflower Oil, Olive Oil, Avocado Oil, Canola Oil, Coconut Oil, Corn Oil, Cottonseed Oil, Flaxseed/Linseed Oil, Grape Seed Oil, Hemp Seed Oil, Palm Oil, Peanut Oil, Rice Bran Oil, Sesame Oil, Soybean Oil, Brazil Nut Oil, Almond Oil, Walnut Oil, and Pecan Oil.

21. The transdermal delivery formulation of any of embodiments 1-20 wherein said plant oil is selected from the group consisting of Macadamia Oil, Maracuja (Passion Fruit) Oil, Safflower Oil, Sunflower Oil, Olive Oil, and Almond Oil.

22. The transdermal delivery formulation of any one of embodiments 1-21 wherein said plant oil is Macadamia Oil or Maracuja (Passion Fruit) Oil.

23. The transdermal delivery formulation of any one of embodiments 1-22 wherein said plant oil comprises Macadamia Oil and Maracuja (Passion Fruit) Oil.

24. The transdermal delivery formulation of any one of embodiments 1-23 wherein said microemulsion is selected from the group consisting of a cream, an ointment, a liniment, a paste, a film, and a liquid.

25. The transdermal delivery formulation of any one of embodiments 1-24 wherein said transdermal accelerant comprises a nitrate source.

26. The transdermal delivery formulation of any one of embodiments 1-25 wherein said nitrate source is a plant-based nitrate source.

27. The transdermal delivery formulation of any one of embodiments 1-26 wherein said plant-based nitrate source is selected from the group consisting of arugula, spinach, and beetroot.

28. The transdermal delivery formulation of any one of embodiments 1-27 wherein said plant-based nitrate source is beetroot.

29. The transdermal delivery formulation of any one of embodiments 1-28, further comprising a (a) a viscosity enhancer, (b) a nutrient, (c) a plant powder or extract, (d) an amino acid, and a (e) vitamin.

30. The transdermal delivery formulation of any one of embodiments 1-29 wherein said formulation comprises a viscosity enhancer selected from the group consisting of lecithin, aloe vera, glycerin, a plant oil, an animal oil, and collagen.

31. The transdermal delivery formulation of any one of embodiments 1-30 wherein said formulation comprises a nutrient selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

32. The transdermal delivery formulation of any one of embodiments 1-31 wherein said formulation comprises a plant powder or extract selected from the group consisting of bacopa powder, bamboo extract powder, beet powder, blueberry extract, ginkgo biloba, ginger, grape seed extract, green tea, jojoba, nutmeg, olive leaf, pomegranate, and turmeric.

33. The transdermal delivery formulation of any one of embodiments 1-32 wherein said formulation comprises an amino acid selected from the group consisting of alanine, arginine, leucine, isoleucine, valine, glutamine, glycine, histidine, leucine, lysine, methionine, proline, serine, threonine, and valine.

34. The transdermal delivery formulation of any one of embodiments 1-33 wherein said formulation comprises a vitamin selected from the group consisting of vitamin A, vitamin B, vitamin B-3, vitamin B-7 & 8 inositol, vitamin B-9 (folic acid), vitamin B-12, vitamin C, vitamin D-3, and vitamin E.

35. The transdermal delivery formulation of any one of embodiments 1-34 wherein said transdermal formulation has a pH of from 2.0 to 6.0.

36. The transdermal delivery formulation of any one of embodiments 1-35 wherein said transdermal formulation has a pH of from 3.0 to 5.0.

37. The transdermal delivery formulation of any one of embodiments 1-36 wherein said transdermal formulation has a pH of from 3.5 to 4.5.

38. The transdermal delivery formulation of any one of embodiments 1-37 wherein said transdermal formulation has a viscosity at 20° C. of from 500 to 10,000 centipoise (cP) or from 1,000 to 5,000 cP, or from 1,500 to 4,000 cP, or from 2,000 to 3,000 cP.

39. The transdermal delivery formulation of any one of embodiments 1-38 wherein said transdermal formulation has a viscosity of about 2,500 cP.

40. A transdermal delivery formulation comprising: a homogenous mixture of (a) a transdermal accelerant comprising a weak organic acid and (b) a microemulsion comprising a nonionic emulsifier, water, and a plant oil comprising an unsaturated long-chain fatty acid.

41. The transdermal delivery formulation of any one of embodiment 40 wherein said weak organic acid has a median pKa of from 2.0 to 6.0.

42. The transdermal delivery formulation of any one of embodiments 40-41 wherein said weak organic acid has a median pKa of from 3.0 to 5.5.

43. The transdermal delivery formulation of any one of embodiments 40-42 wherein said weak organic acid has a median pKa of from 4.0 to 5.0.

44. The transdermal delivery formulation of any one of embodiments 40-43 wherein said weak organic acid has a median pKa of about 4.6.

45. The transdermal delivery formulation of any one of embodiments 40-44 wherein said weak organic acid is selected from the group consisting of lactic acid, acetic acid, formic acid, citric acid, oxalic acid, uric acid, malic acid, maleic acid, tartaric acid, malonic acid, succinic acid, and fumaric acid.

46. The transdermal delivery formulation of any one of embodiments 40-45 wherein said weak organic acid is citric acid or acetic acid.

47. The transdermal delivery formulation of any one of embodiments 40-46 wherein said transdermal accelerant comprises citric acid and acetic acid.

48. The transdermal delivery formulation of any one of embodiments 40-47 wherein said nonionic emulsifier is selected from the group consisting of lecithin, carboxylmethylcellulose, a sorbitan ester, and a polysorbate.

49. The transdermal delivery formulation of any one of embodiments 40-48 wherein said nonionic emulsifier is a sorbitan ester selected from the group consisting of sorbitan monolaurate, sorbitan monostearate, sorbitan tristearate, and sorbitan monooleate.

50. The transdermal delivery formulation of any one of embodiments 40-49 wherein said nonionic emulsifier is a polysorbate selected from the group consisting of polyoxyethylene (20) sorbitan monolaurate (Polysorbate 20), polyoxyethylene (20) sorbitan monopalmitate (Polysorbate 40), polyoxyethylene (20) sorbitan monostearate (Polysorbate 60), and polyoxyethylene (20) sorbitan monooleate (Polysorbate 80).

51. The transdermal delivery formulation of any one of embodiments 40-50 wherein said polysorbate is Polysorbate 80.

52. The transdermal delivery formulation of any one of embodiments 40-51 wherein said water is distilled water.

53. The transdermal delivery formulation of any one of embodiments 40-52 wherein said unsaturated long-chain fatty acid comprises a chain of from 16 to 26 carbons.

54. The transdermal delivery formulation of any one of embodiments 40-53 wherein said 16 to 26 carbon unsaturated long-chain fatty acid comprises one or more double bond in a cis configuration.

55. The transdermal delivery formulation of any one of embodiments 40-54 wherein said 16 to 26 carbon unsaturated long-chain fatty acid is selected from the group consisting of Sapienic Acid, Palmitoleic Acid, Margoleic acid, Cis-Vaccenic Acid, Oleic Acid, Petroselinic Acid, Linoleic Acid, Eicosenoic Acid, Gadoleic Acid, Eicosadienoic acid, Erucic acid, Docosadienoic Acid, and Nervonic acid.

56. The transdermal delivery formulation of any one of embodiments 40-55 wherein said 16 to 26 carbon unsaturated long-chain fatty acid is Oleic Acid or Linoleic Acid.

57. The transdermal delivery formulation of any one of embodiments 40-56 wherein said 16 to 26 carbon unsaturated long-chain fatty acid comprises Oleic Acid and Linoleic Acid.

58. The transdermal delivery formulation of any one of embodiments 40-57 wherein said plant oil is selected from the group consisting of vegetable oil, nut oil and seed oil.

59. The transdermal delivery formulation of any one of embodiments 40-58 wherein said plant oil is selected from the group consisting of Macadamia Oil, Maracuja (Passion Fruit) Oil, Safflower Oil, Sunflower Oil, Olive Oil, Avocado Oil, Canola Oil, Coconut Oil, Corn Oil, Cottonseed Oil, Flaxseed/Linseed Oil, Grape Seed Oil, Hemp Seed Oil, Palm Oil, Peanut Oil, Rice Bran Oil, Sesame Oil, Soybean Oil, Brazil Nut Oil, Almond Oil, Walnut Oil, and Pecan Oil.

60. The transdermal delivery formulation of any one of embodiments 40-59 wherein said plant oil is selected from the group consisting of Macadamia Oil, Maracuja (Passion Fruit) Oil, Safflower Oil, Sunflower Oil, Olive Oil, and Almond Oil.

61. The transdermal delivery formulation of any one of embodiments 40-60 wherein said plant oil is Macadamia Oil or Maracuja (Passion Fruit) Oil.

62. The transdermal delivery formulation of any one of embodiments 40-61 wherein said plant oil comprises Macadamia Oil and Maracuja (Passion Fruit) Oil.

63. The transdermal delivery formulation of any one of embodiments 40-62 wherein said microemulsion is selected from the group consisting of a cream, an ointment, a liniment, a paste, a film, and a liquid.

64. The transdermal delivery formulation of any one of embodiments 40-63 wherein said transdermal accelerant comprises a nitrate source.

65. The transdermal delivery formulation of any one of embodiments 40-64 wherein said nitrate source is a plant-based nitrate source.

66. The transdermal delivery formulation of any one of embodiments 40-65 wherein said plant-based nitrate source is selected from the group consisting of arugula, spinach, and beetroot.

67. The transdermal delivery formulation of any one of embodiments 40-66 wherein said plant-based nitrate source is beetroot.

68. The transdermal delivery formulation of any one of embodiments 40-67, further comprising a (a) a viscosity enhancer, (b) a nutrient, (c) a plant powder or extract, (d) an amino acid, I/or a (e) vitamin.

69. The transdermal delivery formulation of any one of embodiments 40-68 wherein said formulation comprises a viscosity enhancer selected from the group consisting of lecithin, aloe vera, glycerin, a plant oil, an animal oil, and collagen.

70. The transdermal delivery formulation of any one of embodiments 40-69 wherein said formulation comprises a nutrient selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

71. The transdermal delivery formulation of any one of embodiments 40-70 wherein said formulation comprises a plant powder or extract selected from the group consisting of bacopa powder, bamboo extract powder, beet powder, blueberry extract, ginkgo biloba, ginger, grape seed extract, green tea, jojoba, nutmeg, olive leaf, pomegranate, and turmeric.

72. The transdermal delivery formulation of any one of embodiments 40-71 wherein said formulation comprises an amino acid selected from the group consisting of alanine, arginine, leucine, isoleucine, valine, glutamine, glycine, histidine, leucine, lysine, methionine, proline, serine, threonine, and valine.

73. The transdermal delivery formulation of any one of embodiments 40-72 wherein said formulation comprises a vitamin selected from the group consisting of vitamin A, vitamin B, vitamin B-3, vitamin B-7 & 8 inositol, vitamin B-9 (folic acid), vitamin B-12, vitamin C, vitamin D-3, and vitamin E.

74. The transdermal delivery formulation of any one of embodiments 40-73 wherein said transdermal formulation has a pH of from 2.0 to 6.0.

75. The transdermal delivery formulation of any one of embodiments 40-74 wherein said transdermal formulation has a pH of from 3.0 to 5.0.

76. The transdermal delivery formulation of any one of embodiments 40-75 wherein said transdermal formulation has a pH of from 3.5 to 4.5.

77. The transdermal delivery formulation of any one of embodiments 40-76 wherein said transdermal formulation has a viscosity at 20° C. of from 500 to 10,000 centipoise (cP) or from 1,000 to 5,000 cP, or from 1,500 to 4,000 cP, or from 2,000 to 3,000 cP.

78. The transdermal delivery formulation of any one of embodiments 40-77 wherein said transdermal formulation has a viscosity at 20° C. of about 2,500 cP.

79. The transdermal delivery formulation of any of embodiments 1-78 for epicutaneous administration of a drug, nutrient, or other compound to a human subject or domestic, veterinary, or agricultural animal.

80. A transdermal delivery formulation of any of embodiments 1-79 further comprising one or more components for enhancing (1) exercise performance, (2) restful sleep, (3) nitric oxide metabolism, and/or (4) cognitive function.

81. The transdermal delivery formulation for enhancing exercise performance of embodiment 80 further comprising one or more of (1) a hydration electrolyte, (2) cinnamic acid, (3) phenylalanine, (4) resveratrol, and (5) carnitine.

82. The transdermal delivery formulation for enhancing restful sleep of embodiment 80 further comprising one or more of (1) gamma-aminobutyric acid (GABA), (2) melatonin, and (3) tryptophan.

83. The transdermal delivery formulations for enhancing nitric oxide metabolism of embodiment 80 further comprising one or more of (1) vitamin D, (2) vitamin C, (3) citrulline, (4) ribose ATP blood carrier, (5) beetroot, (6) curcumin (turmeric), (7) fish oil, and (8) threonine.

84. The transdermal delivery formulations for enhancing cognitive performance of claim 80 further comprising one or more of silicon, leucine, lysine, isoleucine, valine, threonine, phenylalanine, methionine, histidine, tryptophan, vitamin B-5 (pantothenic acid), vitamin B-7 (biotin), vitamin K, acetyl L carnitine, palmitoyl-ethanolamide (PEA), taurine, periwinkle, artichoke, bacopa, ginkgo biloba, nutmeg, alanine, and tyrosine.

### EXAMPLES

### Example 1: Manufacture of Transdermal Delivery Formulation

An emulsion, also referred to as an emulsified formulation, was generated by combining an acidic composition with oil, water and surfactant. A Polypeptide Transdermal Accelerant (PTA) was made by combining 340 ml (374 g) citric acid powder, 800 ml (640 g) apple cider vinegar, and 60 ml (44.4 g) beetroot powder. A Carrier Formulation was made by combining 700 ml polysorbate 80 (742 g), 700 ml (700 g) distilled water, and, 350 ml (308 g) macadamia oil, and 350 ml (308 g) maracuja oil. The PTA and Carrier Formulation were poured together and mixed for 60-180 minutes at 70-85° F. The pH was checked to confirm acidity of pH 4.5-5.5. Viscosity was confirmed at 2,000-3,000 centipoise (cP). The emulsion was poured in to a fine micro-mesh filter (mesh #325/44 microns) suspended over a 150 oz vat. Emulsion was incubated at room temperature without agitation for 24-36 hours. Surface impurities were skimmed of liquid surface using a 44 micron micro mesh filter.

The emulsion was drained from vat. The pH was measured to confirm pH 3.5-5.5. Viscosity was confirmed at 2,000-3,000 cP.

The Transdermal Delivery Formulation was placed in a refrigerated/cooling storage area, in a range between 37-45 degrees Fahrenheit, until and up to 60 days.

### Example 2: Application of Transdermal Delivery Formulation

As a non-limiting example, A 0.35 oz dose of transdermal delivery formulation as described in Example 1 is applied to an area of skin of a subject. The area of skin to which the formulation is applied is overlaid with ionized water, in liquid or gas form.

### Example 3: In Vitro Models for Testing Transdermal Delivery Formulations

This Example provides in vitro model systems that may be adapted and employed for the testing various aspects of the transdermal delivery formulations disclosed herein.

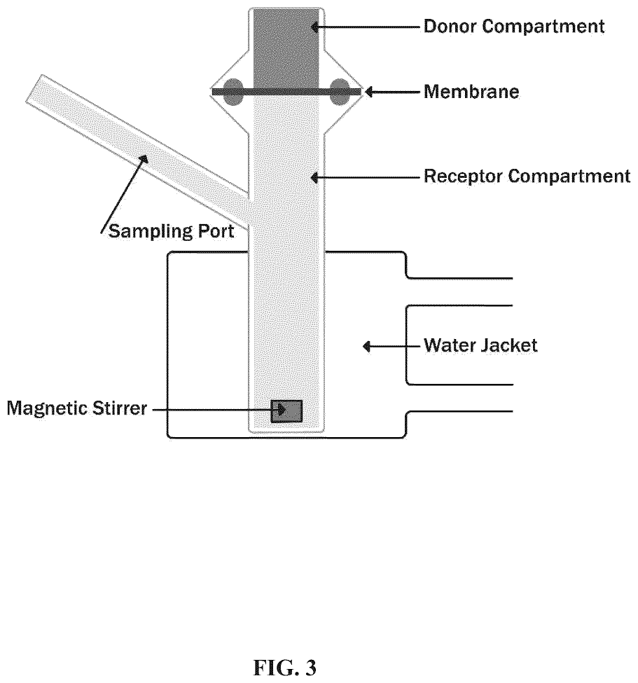

In vitro methods are designed to measure the penetration of compounds, including drugs and nutrients, into the skin and permeation through the stratum corneum and epidermis to the site of vascularization. The Franz diffusion cell (see, FIG. 3) can utilize non-viable skin to measure penetration and permeation only or fresh, metabolically active skin to simultaneously measure permeation and skin metabolism.

Such experiments offer a number of advantages over whole-animal or human volunteer experiments, including saving in time and costs, better reproducibility of results, and less restricted parameter variations. Additional advantages of the in vitro method over the in vivo method are that it can be used equally well with skin from humans and other species, several replicate measurements can be made from the same or number of different subjects, non-radio-labeled test substances which are extensively metabolized can be studied.

In vitro testing is carried out in accordance with “OECD Guideline for the Testing of Chemicals. Draft New Guideline 428: Skin Absorption in vitro method” and Scientific Committee on Consumer Products (SCCP) guidelines.

The in vitro measurement of skin penetration of transdermal delivery formulations includes the application of a test substance in an appropriate formulation (may be radiolabeled) to the surface of a skin sample, which is mounted as a barrier between the donor compartment and the receptor compartment of a diffusion cell. FIG. 3

The majority of skin absorption studies are conducted using horizontal cells, with the skin surface open to the air. The use of vertical (or side-by-side) cells is more common when evaluating drug delivery systems, such as sonophoresis, iontophoresis or electroporation and requires immersion of both surfaces of the skin preparation, which may result in excessive hydration and possibly skin damage.

Diffusion cells include an inert non-adsorbing material with receptor chamber volumes of about 0.5-10 ml and surface areas of exposed membranes of about 0.2-2 cm². Testing is performed with an appropriate number (i.e. minimum six) skin samples.

The receptor fluid, which must have an adequate capacity to solubilize the test substance, is maintained in contact with underside of the skin from the time of application of a transdermal delivery formulation until the end of the collection of the receptor fluid. Temperature control of the receptor fluid is maintained and monitored throughout the testing. The skin surface temperature in the diffusion cell should be kept at the in vivo skin temperature of 32±1° C. The receptor fluid in static cells is well-stirred throughout the study.

### Example 4: Observational Study with Metabolic Transdermal Delivery Formulation

This Example provides evidence for the therapeutic benefits of a metabolic transdermal delivery formulation through in vivo data presented in Tables 7 and 8 that demonstrate improvements in three key indicia of human health: weight, heart rate, and blood pressure following administration of a transdermal delivery formulation as disclosed herein. Five (5) participants applied to their umbilicus (navel area) approximately 0.35 oz of a Metabolic Transdermal Delivery Formulation twice daily for 21 consecutive days. Each participant's weight, heart rate, and blood pressure were measured on days 0, 7, 14, and 21 and Blood Pressure and Heart Rate were measured.

TABLE 7

Blood Pressure

| Timing | Part 1 | Part 2 | Part 3 | Part 4 |
|---|---|---|---|---|
| Day 0 | 175/86 | 107/72 | 126/76 | 145/75 |
| Day 7 | 153/98 | 94/69 | 113/74 | 128/78 |
| Day 14 | 153/87 | 113/79 | 102/66 | 145/79 |
| Day 21 | 153/98 | 117/69 | | 134/74 |

TABLE 8

Heart Rate

| Timing | Part 1 | Part 2 | Part 3 | Part 4 |
|---|---|---|---|---|
| Day 0 | 57 | 68 | 61 | 58 |
| Day 7 | 70 | 72 | 61 | 52 |
| Day 14 | 64 | 73 | 58 | 58 |
| Day 21 | 64 | 61 | | 57 |

### Example 5: Observational Study with a Transdermal Delivery Formulation

This Example provides evidence for the therapeutic benefits of a transdermal delivery formulation as disclosed herein though in vivo data that demonstrates cognitive improvements in five (5) participants from 60 to 79 years of age and with no previous diagnosis of dementia who applied a transdermal delivery formulation to the nape (nucha) of the neck and around the entirety of the neck area approximately 0.25 oz of a Focus™ Transdermal Delivery Formulation (or Placebo Control), as described in Table 6, once daily for 28 consecutive days.

Improved cognitive function and performance were demonstrated through studies performed by BrainCheck. Inc. (Austin, TX). Two protocols are used during the study. (1) TNS Studies' FOCUS Serum, (2) BrainCheck's online cognitive assessment.

Baseline testing is captured using BrainCheck's online cognitive assessment tool. BrainCheck performed an online cognitive assessment one each participant and provided testing results.

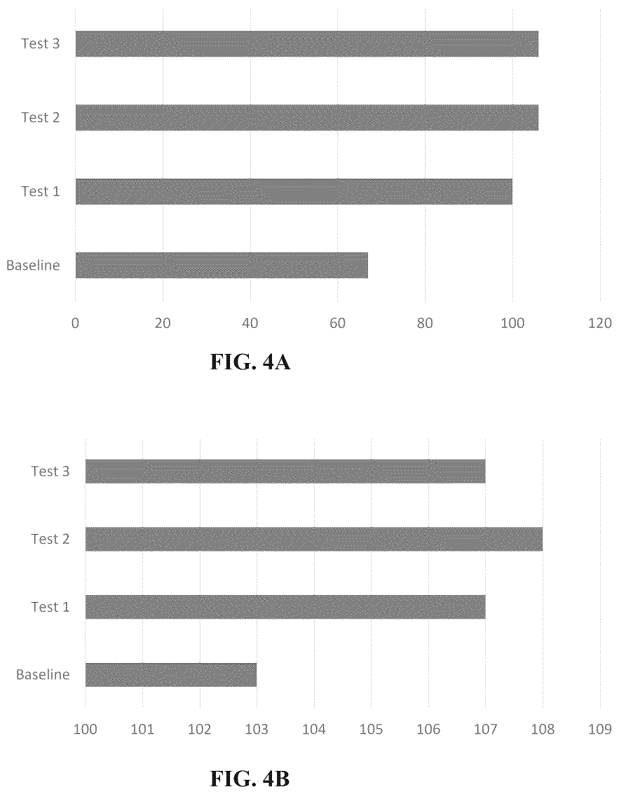

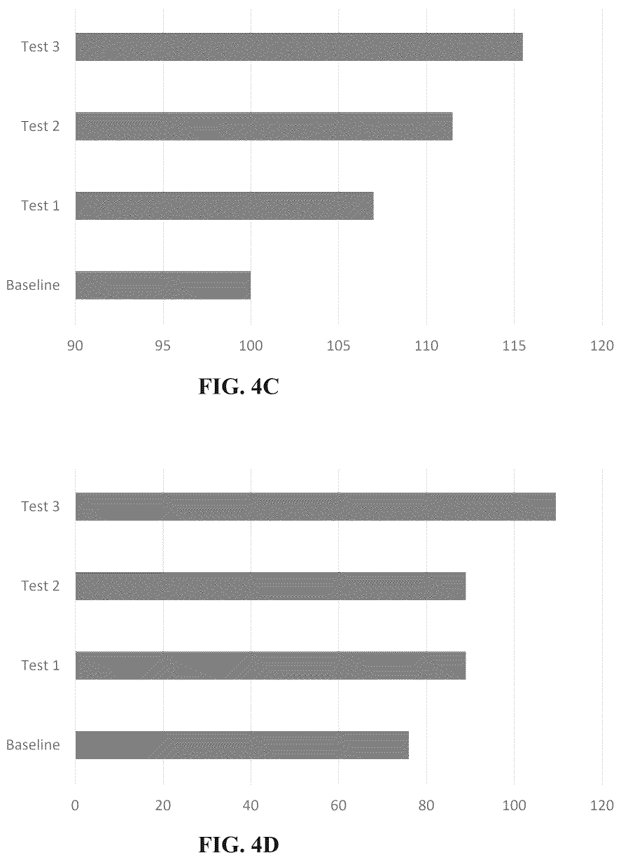

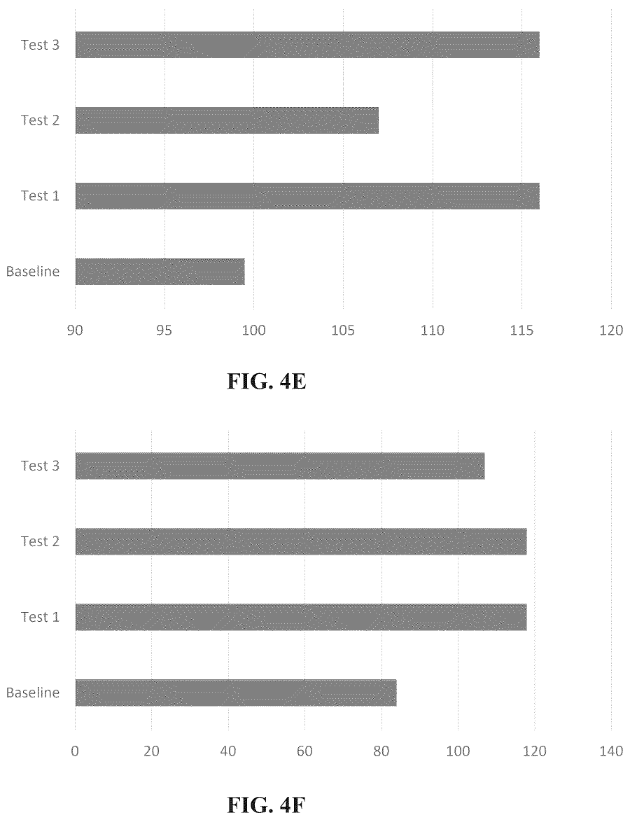

The results of this study are presented in Table 9 and FIGS. 4A-4F. The average change in Total Score from baseline to Test 3, as reported by BrainCheck, was 10% (data not shown). Improvements in executive function were demonstrated by a 27% improvement from baseline for Stroop Color Interference measurements of the ability of a human subject or domestic, veterinary, or agricultural animal to inhibit reactions and control impulses.

As depicted in FIGS. 4A-F, all test groups shows an increase in score over baseline. Test subjects in the 51-60 age range showed the greatest improvement in Total Score, 19.5%. Attention improved by 29%, Mental Flexibility improved by 4% (Ages 71-80), Executive Function (digital symbol substitution) improved by 15.5% (Ages 41-50), Executive Function (stroop) improved by 44% (Ages 41-50), Immediate Memory improved by 16.6% (Ages 51-60), and Delayed Memory improved by 27% (Ages 71-80).

TABLE 9
Attention
Trails A

| | |
|---|---|
| Baseline | 67 |
| Test 1 | 100 |
| Test 2 | 106 |
| Test 3 | 106 |

Mental Flexibility
Trails B

| | |
|---|---|
| Baseline | 103 |
| Test 1 | 107 |
| Test 2 | 108 |
| Test 3 | 107 |

Executive Function
Digital Symbol Substitution

| | |
|---|---|
| Baseline | 100 |
| Test 1 | 107 |
| Test 2 | 111.5 |
| Test 3 | 115.5 |

Executive Function
Stroop

| | |
|---|---|
| Baseline | 76 |
| Test 1 | 89 |
| Test 2 | 89 |
| Test 3 | 109.5 |

Memory
Immediate Recognition

| | |
|---|---|
| Baseline | 99.5 |
| Test 1 | 116 |
| Test 2 | 107 |
| Test 3 | 116 |

Memory
Delayed Recognition

| | |
|---|---|
| Baseline | 84 |
| Test 1 | 118 |
| Test 2 | 118 |
| Test 3 | 107 |

### Example 6: Observational Study with a Transdermal Delivery Formulation

This Example provides evidence for the therapeutic benefits of a transdermal delivery formulation as disclosed herein though in vivo data that demonstrates cognitive improvements in eight (8) participants from 34 to 72 years of age and with no previous diagnosis of dementia who applied to the nape of the neck area approximately 0.25 oz of a Focus™ Transdermal Delivery Formulation (or Placebo Control), as described in Table 6, once daily for 28 consecutive days.

Improved cognitive function and performance were demonstrated through studies performed using RC21X by Home Base Impairment Company, Inc. (Coraopolis, PA). Two protocols were used during the study: (1) TNS Studies' FOCUS Serum, and (2) RC21X's ROBERTO mobile application.

Baseline testing was captured using RC21X's ROBERTO mobile application. RC21X provided testing results on each participants' test.

with the Roberto App (RC21X, Coraopolis, PA). Testing scores were tracked and indicators of improvement were assessed in the following areas: (1) mental focus, (2) hand/eye coordination, (3) eye tracking, (4) auditory and visual recognition & recollection, and (5) reduction in stress and mental fatigue. 100% of Study Participants reported improved or less stress during the Study. 83% of Study Participants' sleep was improved. 66% of Study Participants reported feeling calmer while on the Serum.

The results of this study are presented in Table 10. Improvements in average scores across five (5) of the six (6) tests were demonstrated from baseline to the last test performed. Visual discrimination and impulse control (Base-Stealing Game) showed the greatest overall improvement as an average of all participant scores. The 61-70 age group demonstrated improvement in four (4) of the five (5) tests. Visual memory delayed recall (Video Recognition) demonstrated a 38% increase in the 41-50 age group. Audio memory delayed recall (Audio Recognition), Bilateral finger motor speed (Finger Tapping), Working memory and visual attention (Shape Memory) and Visual discrimination and impulse control (Base-Stealing Game) demonstrated 56%, 46%, 126% and 375% respectively, improvement from baseline to last test in the 61-70 age group. Working memory/visual attention (Number Memory), a 29% improvement, was demonstrated in the 71-80 age group.

TABLE 10
Results of ROBERTO test.

| | | | |
|---|---|---|---|
| Part # | Baseline | Last Test | % Change |

Visual Memory Delayed Recall
(Video Recognition)

| | | | |
|---|---|---|---|
| 4150-JM | 63 | 87 | 38.10% |

Audio Memory Delayed Recall
Audio Recognition

| | | | |
|---|---|---|---|
| 6170-LI | 23 | 42 | 82.61% |
| 6170-IC | 26 | 48 | 84.62% |
| 6170-LaI | 41 | 42 | 2.44% |

Bilateral Finger Motor Speed
Finger Tapping

| | | | |
|---|---|---|---|
| 6170-LI | 57 | 118 | 107.02% |
| 6170-IC | 57 | 62 | 8.77% |
| 6170-LaI | 59 | 72 | 22.03% |

Working Memory and Visual Attention
Shape Memory

| | | | |
|---|---|---|---|
| 6170-LI | 15 | 70 | 366.67% |
| 6170-IC | 49 | 52 | 6.12% |
| 6170-LaI | 45 | 48 | 6.67% |

Working Memory and Visual Attention
Number Memory

| | | | |
|---|---|---|---|
| 7180-JH | 45 | 70 | 55.56% |
| 7180-RH | 52 | 71 | 36.54% |
| 7180-SF | 71 | 67 | −5.63% |

Visual Discrimination and Impulse Control
Base-Stealing

| | | | |
|---|---|---|---|
| 6170-LI | 6 | 71 | 1083.33% |
| 6170-IC | 56 | 72 | 28.57% |
| 6170-LaI | 71 | 81 | 14.09% |

### Example 7: Characterization of Transdermal Delivery Formulation

Transdermal Delivery Formulations were characterized using emulsion droplet size and zeta sizer analysis. Three batches of emulsion (designated batch 1, 2, and 3) were generated as described in Example 1. Emulsion was held for 24 hours at 37-45 degrees C. Duplicate samples (designated A and B) were taken from each batch for testing.

### Zetasizer Analysis

Samples were diluted in HPLC-grade water at 1:200 and 1:400. Additionally, to compare results from samples diluted in local (Mobile, AL) tap water, a sample from Batch A was prepared by diluting 1:1 in tap water, then further diluted in tap water or HPLC-grade water to a final dilution of 1:200 or 1:400.

Sizing measurements were made using a Zetasizer Nano series instrument (Malvern Panalytical Inc., Westborough, MA). Three measurements were carried out for each sample. Eleven runs or cycles were completed for each replicate. Each run had a duration of 10 seconds. Zetasizer settings were as follows:
Temperature: 25° C.
Material: Nanoparticles
Material refractive index (RI): 1.00
Dispersant name: Water
Dispersant RI: 1.330
Viscosity (cP): 0.8888
Equilibration time: 1 min
Cell Description: Disposable sizing cuvette

### Zetapotential Analysis and Count Rate

Zeta potential and count rate were measured using a Zetaview® instrument (Particle Metrix, Ammersee, Germany). Samples were diluted 1:100,000 in water. Analysis was performed with the following settings:
Temperature: 25° C.
Electrolyte: Water
Laser: 488 nm
Filter Wavelength: Scatter
Cell S/N: ZNTA
Sensed Electric Field: 3.39 V/cm (pulse)
Measurement Mode: Stationary 3 cycles

Size, count rate, and zeta potential data are shown in Table 11.

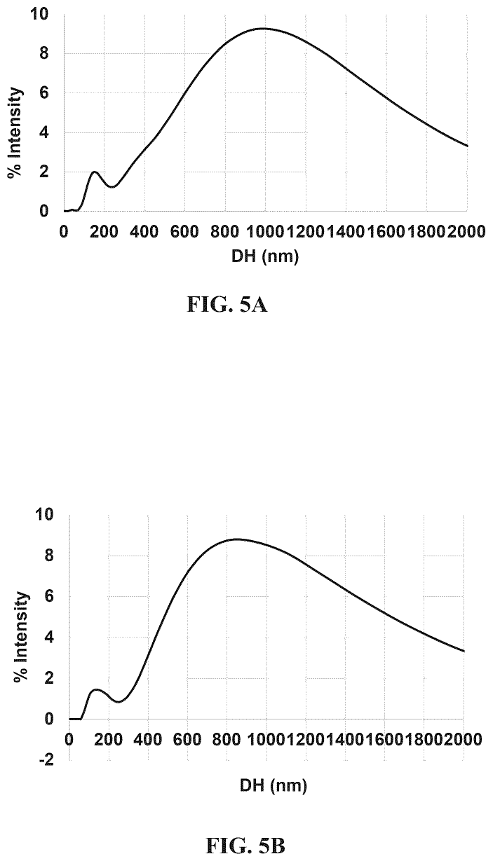

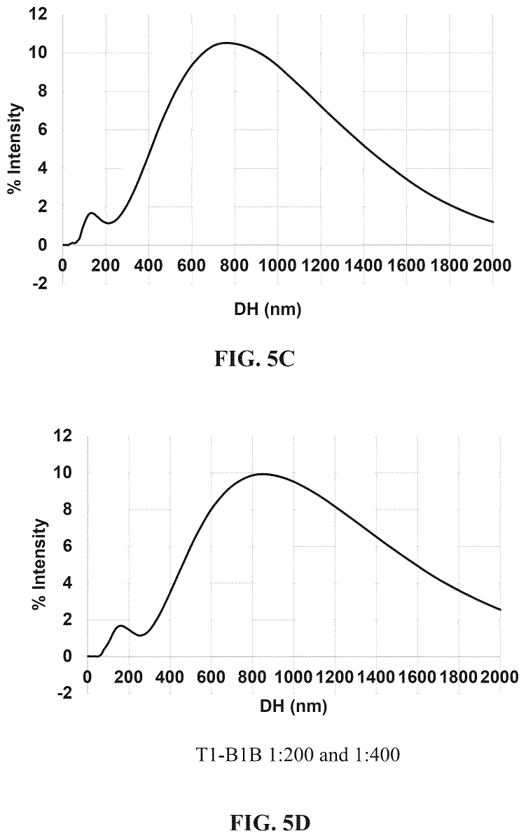

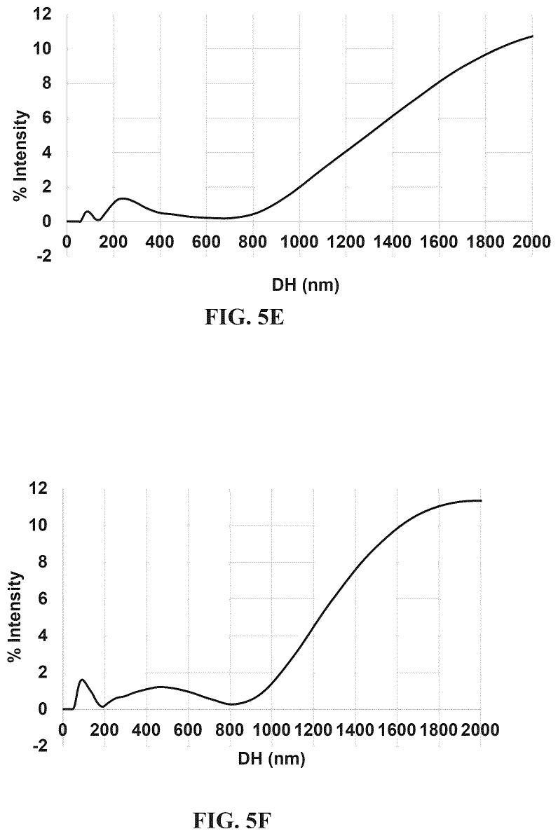

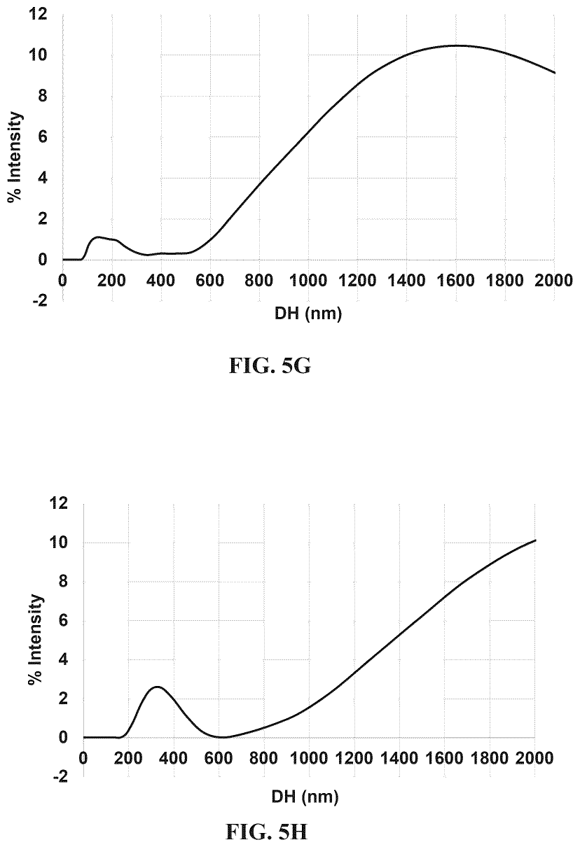

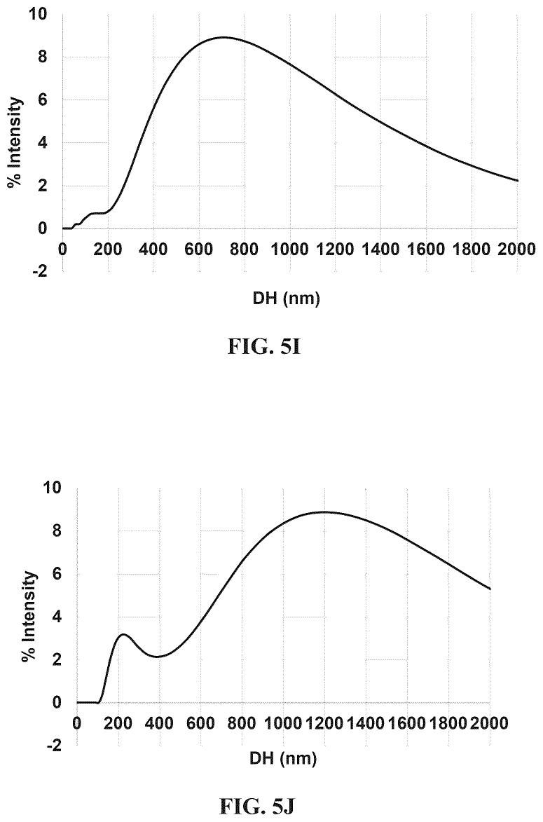

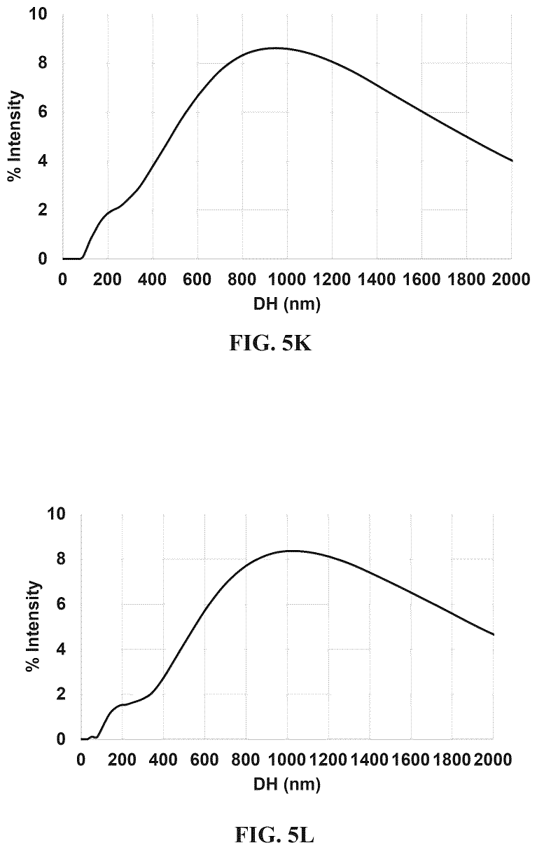

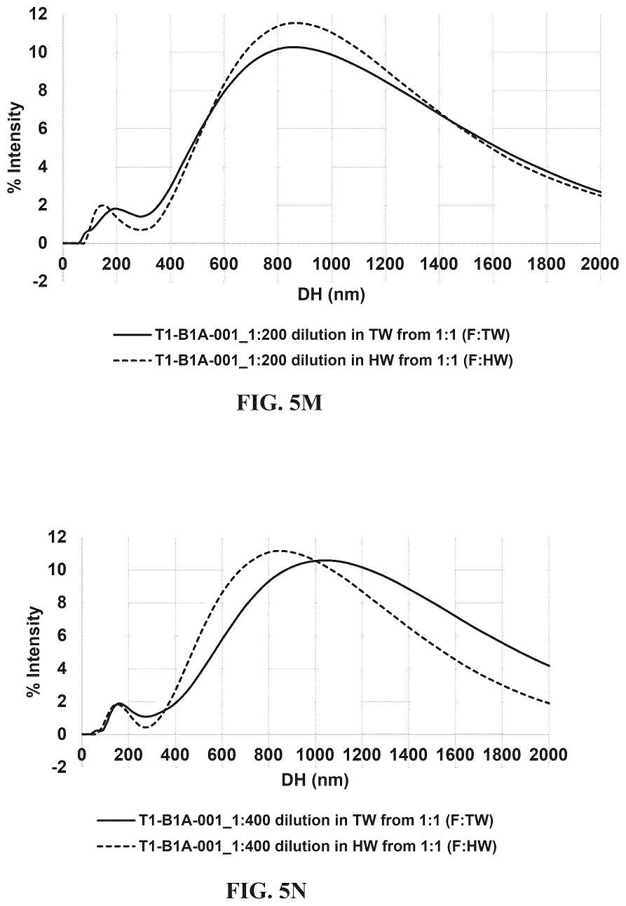

TABLE 11
Characterization data from batches of Transdermal Delivery Formulation

| | | | | | Count rate | Zeta potential |
|---|---|---|---|---|---|---|
| | Dilution ratios | Size | | | (kilo counts | (mV) |
| | (Formulation: | distributions | Mode | | per second - | 1:100,000 |
| Sample | HPLC H₂O) | (nm) | (nm) | FIG. | kcps) | dilution |
| Batch 1 - A | 1:200 | 68-255 | 164 | FIG. 5A | 280.83 | −16.6 |
| | | 255-5560 | 955 | | | |
| | 1:400 | 68-255 | 142 | FIG. 5B | 200.43 | |
| | | 255-3580 | 825 | | | |
| | | 3580-5560 | 5560 | | | |
| Batch 1 - B | 1:200 | 68-220 | 141 | FIG. 5C | 327.03 | −21.32 |
| | | 220-2669 | 825 | | | |
| | | 2669-4801 | 4801 | | | |
| | 1:400 | 68-295 | 164 | FIG. 5D | 357.43 | |
| | | 295-3580 | 825 | | | |
| | | 3580-5560 | 5560 | | | |
| Batch 2 - A | 1:200 | 68-122 | 91 | FIG. 5E | 440.73 | −17.19 |
| | | 141-615 | 255 | | | |
| | | 712-5560 | 2305 | | | |
| | 1:400 | 51-190 | 91 | FIG. 5F | 340.56 | |
| | | 190-825 | 459 | | | |
| | | 825-4145 | 1990 | | | |
| | | 4145-5560 | 5560 | | | |
| Batch 2 - B | 1:200 | 91-358 | 141 | FIG. 5G | 329.36 | −27.59 |
| | | 358-4145 | 1718 | | | |
| | | 4145-5560 | 5560 | | | |
| | 1:400 | 190-531 | 342 | FIG. 5H | 287.43 | |
| | | 712-5560 | 2305 | | | |
| Batch 3 - A | 1:200 | 79-190 | 122 | FIG. 5I | 265.93 | −21.45 |
| | | 190-3091 | 712 | | | |
| | | 3091-5560 | 5560 | | | |
| | 1:400 | 122-396 | 220 | FIG. 5J | 231.63 | |
| | | 396-5560 | 1281 | | | |
| Batch 3 - B | 1:200 | 122-3580 | 955 | FIG. 5K | 430.66 | −23.95 |
| | | 3580-5560 | 5560 | | | |
| | 1:400 | 79-5560 | 1106 | FIG. 5L | 275.53 | |
| Dilutions | | | | | | |
| in tap water | | | | | | |
| Batch 1- A | 1:200 dilution | 68-295 | 220 | FIG. 5M | 206.53 | |
| | in TW from 1:1 | 295-4145 | 825 | | | |
| | (F:TW) | 4145-5560 | 5560 | | | |
| | 1:400 dilution | 91-295 | 164 | FIG. 5N | 129.26 | |
| | in TW from 1:1 | 295-4145 | 1106 | | | |
| | (F:TW) | | | | | |
| | 1:200 dilution | 79-164 | 141 | FIG. 5M | 216.93 | |
| | in HW from | 164-4145 | 825 | | | |
| | 1:1 (F:TW) | 4145-5560 | 5560 | | | |
| | 1:400 dilution | 43-91 | 68 | FIG. 5N | 118.66 | |
| | in HW from | 91-5560 | 1281 | | | |
| | 1:1 (F:TW) | | | | | |
| | 1:200 dilution | 91-295 | 142 | N/S | 227.06 | |
| | in HW from | 295-3091 | 825 | | | |
| | 1:1 (F:HW) | | | | | |

Line graphs showing size distribution in the sub-2 micron range of each sample preparation are provided in FIGS. 5A-5N. As shown in the figures, all samples comprised a population of emulsion droplets with a diameter from about 10 nm to about 300 nm.

### Example 8: Manufacture of a Molecular Application Platform

A Molecular Application Platform (MAP) emulsion is generated by preparing separate aqueous and oil phases. The aqueous phase is made by combining 80 g citric acid (CAS 77-92-9, MilliporeSigma, Burlington, MA) with 80 g water (CAS 7732-18-5, MilliporeSigma, Burlington, MA) and 0.8 g glacial acetic acid (CAS 64-19-7, MilliporeSigma, Burlington, MA). The composition is heated to 40 degrees C. and stirred at 500 RPM for 5 min. 20 g citrulline and 20 g ascorbic acid are combined as dry powders and added to the water mixture at 40 degrees C. and stirred for 5 minutes at 500 RPM.

The oil phase is made by combining 20 g safflower oil (CAS 8001-23-8, MilliporeSigma, Burlington, MA) with 9 g oleic acid (CAS 112-80-1, MilliporeSigma, Burlington, MA), 3.17 g glycerin (CAS 56-81-5, MilliporeSigma, Burlington, MA), and 80 g polysorbate 80 (CAS 9005-65-6) at 25 degrees C. The oil-based composition is mixed for 5 min at 1000 RPM.

The oil phase is drizzled into the aqueous phase. After adding about one third of the oil phase, the mixing speed is increased to 1000 RPM. After two thirds of the oil phase are added, the mixing speed is increased to between 1500-2500 RPM. The emulsion is stirred at 40 degrees C. for an additional 5-30 minutes. pH and viscosity are measured after stirring the emulsion.

### Example 9: Systemic Delivery of Caffeine by Topical Application

Systemic delivery of active agents in an exemplary beetroot/caffeine emulsion according to the present disclosure was demonstrated, to achieve increased nitric oxide levels out to over 24 hours as a demonstration of a systemic NO-increasing emulsion. A test emulsion having the components of Table 12 was prepared. The formulation for this emulsion included beetroot powder as a nitric oxide precursor, macadamia oil as a source of oleic acid, maracuja oil as a source of linoleic acid, apple cider vinegar as a source of acetic acid, and polysorbate 80 as the emulsifier.

The formulation was prepared by adding the components in the following order: Distilled water, macadamia oil, maracuja oil, beetroot powder, apple cider vinegar and citric acid powder. Then these components were mixed for 20 minutes in a KitchenAid mixer at approximately 600 rpm, before adding polysorbate 80 to the formulation. After addition of polysorbate 80 to the formulation, an emulsion was prepared using a standard operating procedure as follows: The formulation was mixed using the KitchenAid mixer at approximately 600 rpm for 4 to 6 hours, with stoppages for cooling (approx. 2 stoppages) to form the test emulsion with the Table 12 components. The mixing was performed without temperature control.

A 13% w/w caffeine composition/emulsion/NO supplement was generated by adding 0.65 g caffeine powder to 4.35 g to the emulsion comprising the Table 12 components.

TABLE 12
Delivery Emulsion

| Ingredient | mL | g |
|---|---|---|
| Citric Acid Powder | 800 | 880 |
| Apple Cider Vinegar | 1200 | |
| Beetroot Powder | 120 | 88.8 |
| Polysorbate 80 | 800 | |
| Distilled Water | 700 | |
| Macadamia Nut Oil | 150 | |
| Maracuja Oil | 600 | |

2.47 g of the 13% caffeine emulsion, representing 321 mg caffeine was applied to the skin over the abdomen of a subject following 8 hours of fasting. Blood pressure, heart rate, blood oxygen, and salivary nitric oxide content were tracked for 12½ hours. Salivary nitric oxide was tested using nitric oxide indicator strips (Changchun Merydi Bio-Tech Co., Ltd., Changchun, Jilin, China). Nitric oxide indicator strips were compared to a color gauge representing the values as in Table 13.

TABLE 13
Detected NO values with NO Indicator Strips

| Color | Approximate mg/L | Range |
|---|---|---|
| White/light pink | 10 | Depleted |
| Pink | 20 | Low |
| Deep Pink | 110 | Threshold |
| Red | 220 | Target |
| Dark Red | 435 | High |
| Burgundy | 870 | Very high |

The measured levels for various physiological parameters are shown in Table 14.

TABLE 14
Physiologic assessments after application
of the emulsion with beetroot and caffeine

| Mins | BPS | BPD | HR | OX | NO test strip | Observations | Notes |
|---|---|---|---|---|---|---|---|
| 0 | 129 | 80 | 59 | 95 | Depleted | 8 Hours fasting | |
| 6 | 110 | 68 | 64 | 96 | Low | | |
| 14 | 99 | 70 | 57 | 95 | Threshold | | |
| 22 | 101 | 72 | 56 | 97 | High | | |
| 27 | 100 | 77 | 55 | 96 | High | | |
| 34 | 105 | 78 | 56 | 96 | High | | |
| 46 | 111 | 75 | 55 | 94 | High | | |
| 56 | 112 | 78 | 60 | 96 | High | | |
| 86 | 121 | 71 | 65 | 96 | High | HEAD ACHE, | |
| | | | | | | KIDNEYS | |
| 146 | 130 | 71 | 66 | 96 | High | | |
| 236 | 131 | 81 | 71 | 95 | High | BUZZ SLIGHT | Tylenol and B12 |
| | | | | | | HA | taken. |
| 296 | 138 | 78 | 69 | 95 | Target | BUZZ NO | |
| | | | | | | HEADACHE | |
| 356 | 131 | 79 | 65 | 95 | Target | BUZZ NO | Nitric Oxide |
| | | | | | | HEADACHE | remains elevated |
| 446 | 127 | 79 | 61 | 96 | Target | BUZZ NO | Nitric Oxide |
| | | | | | | HEADACHE | remains elevated |
| 566 | 125 | 81 | 60 | 95 | Target | Normal | Nitric Oxide |
| | | | | | | | remains elevated |
| 836 | 127 | 79 | 61 | 96 | Target | Normal | Nitric Oxide |
| | | | | | | | remains elevated |
| 1316 | 127 | 84 | 59 | 96 | Target | Normal | Nitric Oxide |
| | | | | | | | remains elevated |
| 1526 | 105 | 68 | 60 | 96 | Target | Normal | Nitric Oxide |
| | | | | | | | remains elevated |

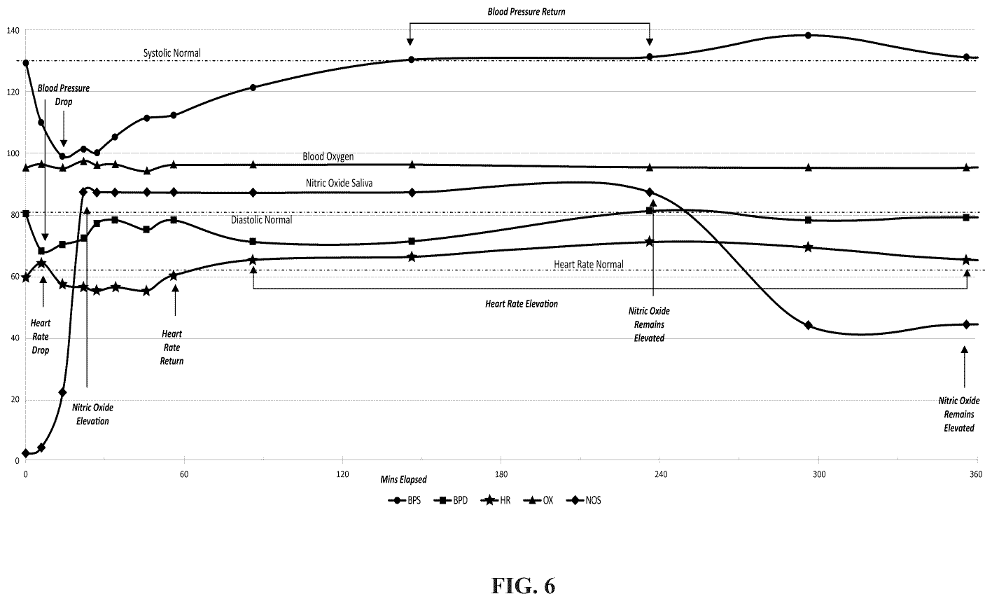

FIG. 6 is a line graph depiction of the results from Table 14 with more quantitative details re: NO levels and comments regarding results for various physiological parameters. As observed, and as a proof-of-concept demonstration of the systemic delivery capability of the emulsions provided herein, a number of physiological parameters were affected by administration of the beetroot/caffeine emulsion that included beetroot as a plant-based, nitrate source, nitric oxide-promoting agent and caffeine as an agent or additional compound, with known physiological effects.

Notably, the physical effects of caffeine delivery, “buzz” and headache, were observed and coincide with an elevated blood pressure and heartrate (Table 14), consistent with the emulsion delivering systemic caffeine to the subject. On the other hand, shortly after administration of the beetroot/caffeine emulsion, both systolic and diastolic blood pressure, as well as heart rate after an early, short-lived minor increase, decreased such that by 14 minutes after administration, the decrease was observed. Such blood pressure and heart rate decrease supports that active agent(s) in addition to caffeine are being delivered with topical administration of the emulsion. Similar reduced heart rate and blood pressure has been observed by the inventors during similar time periods after administration for multiple subjects who have topically administered various test emulsions that include one or more hydrophilic nitric oxide promoting agents such as beetroot powder, citrulline, or citrulline malate, a source of oleic acid, a source of linoleic acid, citric acid, and polysorbate 80 as the emulsifier (data not shown). It is also noteworthy that blood oxygen remained steady throughout the time periods tested.

As shown in the table and FIG. 6, NO levels increased to “High” within 22 minutes. This level was maintained for four hours. As shown in Table 14, NO levels were still elevated above baseline at 1526 minutes (25:26) after administration. This demonstrates systemic delivery of nitric oxide-increasing compounds in the beetroot agent (i.e. load components) in the emulsion. Elevated nitric oxide levels are maintained after 24 hours suggesting that daily, or 2 times, or 2 to 4 times a day dosing would be effective to maintain elevated nitric oxide levels over multiple days.

### Example 10: Nitric Oxide Generation with Daily Supplementation

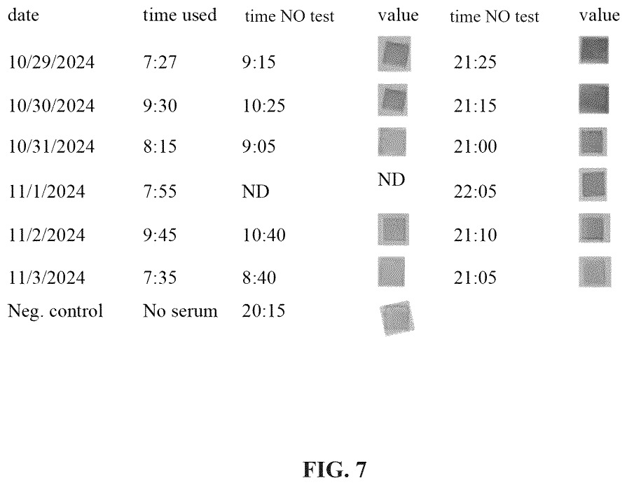

An emulsion was applied at the time points in “time used” column in FIG. 7. Nitric oxide was measured using Nitric Oxide Indicator Strips (Changchun Merydi Bio-Tech Co., Ltd., Changchun, Jilin, China). Resulting NO levels are shown in “value” columns of FIG. 7, with timepoints noted as “time NO test” taken 1-2 hours and 12-14 hours after application of emulsion. Darker pink strips indicate higher levels of NO relative to a baseline value shown. Results show elevated levels of NO 12-14 hours after application of the emulsion with NO supplements. This shows the emulsion provides for maintained elevated levels of nitric oxide for at least 12 hours.

### Example 11: Manufacture of a Molecular Application Platform with Additional Compounds

An MAP emulsion with additional compounds was generated by preparing separate aqueous and oil phases. The aqueous phase was made by combining 80 g citric acid (CAS 77-92-9, MilliporeSigma, Burlington, MA) with 80 g water (CAS 7732-18-5, MilliporeSigma, Burlington, MA) and 0.8 g glacial acetic acid (CAS 64-19-7, MilliporeSigma, Burlington, MA). The composition is heated to 38 degrees C. and stirred at 500 RPM for 20 min.

A composition was generated with 8 g citrulline malate (CAS 77-92-9), 8 g beetroot powder (CAS 89957-89-1), 4 g arginine (CAS 74-79-3), 4 g creatine (CAS 57-00-1), 4 g serine (CAS 56-45-1), 8 g glutathione (CAS 70-18-8), 4 g kale (CAS 89958-13-4), 4 g EEA (CAS X002937WET), and 4 g lecithin (SF) (CAS 8002-43-5) at 25 degrees C. and stirred for 5 minutes. The composition was added to the aqueous phase with stirring at 1000 RPM and 38 degrees C. for 5 minutes. The aqueous composition was further stirred at 1000 RPM and 38 degrees C. for 5 minutes.

The oil phase was made by combining 20 g safflower oil (CAS 8001-23-8, MilliporeSigma, Burlington, MA), 8 g almond oil (CAS 8007-69-0), 16 g avocado oil (CAS 8024-32-6), 8 g glycerin (CAS 56-81-5, MilliporeSigma, Burlington, MA), 9 g oleic acid (CAS 112-80-1, MilliporeSigma, Burlington, MA), and 120 g polysorbate 80 (CAS 9005-65-6) at 25 degrees C. The oil-based composition was stirred for 5 min at 25 degrees C.

The oil phase was drizzled in to the aqueous phase at 38 degrees C. while stirring at 1000 RPM for 5 minutes, generating an emulsion. The emulsion was further stirred at 38 degrees C. for 5 minutes at 1500 RPM.

After cooling the emulsion to 25 degrees C., the pH was measured to be 2.58.

### Example 12: Manufacture of a Base Molecular Application Platform

A base MAP emulsion was generated by preparing separate aqueous and oil phases. The aqueous phase was made by combining 40 g citric acid (CAS 77-92-9, MilliporeSigma, Burlington, MA) with 40 g water (CAS 7732-18-5, MilliporeSigma, Burlington, MA) and 0.4 g glacial acetic acid (CAS 64-19-7, MilliporeSigma, Burlington, MA). The composition is heated to 38 degrees C. and stirred at 1000 RPM for 5 min.

The oil phase was made by combining 10 g safflower oil (CAS 8001-23-8, MilliporeSigma, Burlington, MA), 4.5 g oleic acid (CAS 112-80-1, MilliporeSigma, Burlington, MA), 1.58 g glycerin (CAS 56-81-5, MilliporeSigma, Burlington, MA), and 40 g polysorbate 80 (CAS 9005-65-6) at 25 degrees C. The oil-based composition was stirred for 5 min at 25 degrees C.

The oil phase was drizzled in to the aqueous phase at 38 degrees C. while stirring at 1000 RPM for 5 minutes, generating an emulsion. The emulsion was further stirred at 38 degrees C. for 5 minutes at 2500 RPM.

After cooling the emulsion to 21.5 degrees C., the pH was measured to be 1.89.

### Example 13: Manufacture of a Molecular Application Platform with Additional Caffeine and Kale

An MAP emulsion with additional compounds including caffeine and kale for GLP-1 was generated by preparing separate aqueous and oil phases. The aqueous phase was made by combining 80 g citric acid (CAS 77-92-9, MilliporeSigma, Burlington, MA) with 80 g water (CAS 7732-18-5, MilliporeSigma, Burlington, MA) and 0.8 g glacial acetic acid (CAS 64-19-7, MilliporeSigma, Burlington, MA). The composition is heated to 38 degrees C. and stirred at 500 RPM for 20 min.

A composition was generated with 8 g citrulline malate (CAS 77-92-9), 8 g ascorbic acid (CAS 50-81-7), 8 g beetroot powder (CAS 89957-89-1), 4 g arginine (CAS 74-79-3), 4 g lecithin (SF) (CAS 8002-43-5), 4 g EEA (CAS X002937WET), and 4 g caffeine (CAS 58-08-2) at 25 degrees C. and stirred for 5 minutes. The composition was added to the aqueous phase with stirring at 1000 RPM and 38 degrees C. for 5 minutes. The aqueous composition was further stirred at 1000 RPM and 38 degrees C. for 5 minutes.

The oil phase was made by combining 20 g safflower oil (CAS 8001-23-8, MilliporeSigma, Burlington, MA), 9 g oleic acid (CAS 112-80-1, MilliporeSigma, Burlington, MA), 3.17 g glycerin (CAS 56-81-5, MilliporeSigma, Burlington, MA), and 80 g polysorbate 80 (CAS 9005-65-6) at 25 degrees C. The oil-based composition was stirred for 5 min at 25 degrees C.

The oil phase was drizzled in to the aqueous phase at 38 degrees C. while stirring at 1000 RPM for 5 minutes, generating an emulsion. The emulsion was further stirred at 38 degrees C. for 5 minutes at 2000 RPM.

### Example 14: Manufacture of a Molecular Application Platform with Additional Compounds

An MAP emulsion with additional compounds was generated by preparing separate aqueous and oil phases. The aqueous phase was made by combining 400 g citric acid (CAS 77-92-9, MilliporeSigma, Burlington, MA) with 400 g water (CAS 7732-18-5, MilliporeSigma, Burlington, MA). The composition is heated to 38 degrees C. and stirred at 500 RPM for 20 min.

A composition was generated with 40 g citrulline (CAS 372-75-8), 40 g ascorbic acid (CAS 50-81-7), 20 g beetroot powder (CAS 89957-89-1), 12 g creatine (CAS 57-00-1), 12 g glutathione (CAS 70-18-8), 12 g arginine (CAS 74-79-3), 12 g EEA (CAS X002937WET), 12 g leucine (CAS 61-90-5), 4 g calcium beta-hydroxy-beta-methylbutyrate (HMB) (CAS 135236-72-5), 12 g caffeine (CAS 58-08-20) and 5 g xanthan gum (CAS 11138-66-2) at 25 degrees C. and stirred for 5 minutes. The composition was added to the aqueous phase with stirring at 1000 RPM and 38 degrees C. for 5 minutes. The aqueous composition was further stirred at 1000 RPM and 38 degrees C. for 5 minutes.

The oil phase was made by combining 100 g safflower oil (CAS 8001-23-8, MilliporeSigma, Burlington, MA), 45 g oleic acid (CAS 112-80-1, MilliporeSigma, Burlington, MA), 10 g glycerin (CAS 56-81-5, MilliporeSigma, Burlington, MA), and 400 g polysorbate 80 (CAS 9005-65-6) at 25 degrees C. The oil-based composition was stirred for 5 min at 25 degrees C.

The oil phase was drizzled in to the aqueous phase at 38 degrees C. while stirring at 1000 RPM for 5 minutes, generating an emulsion. The emulsion was further stirred for 29 minutes at 2500 RPM.

### Example 15: Clinical Trial to Evaluate Pharmacokinetics and Nitric Oxide Generation of a Corplex™ Donepezil 10 mg MAP Emulsion

A randomized, open-label, 3-way crossover study with up to 66 healthy, adult male and female subjects are enrolled. All subjects receive LA001, Corplex Donepezil in an MAP emulsion as described in Example 8, applied to the abdomen.

During treatment period, all subjects receive a once-weekly 10 mg LA001, target dose 10 mg donepezil/day, applied for 7 days (1 week) on the abdomen.

Blood samples for donepezil PK are collected pre-dose until the end of each treatment period.

Nitric oxide levels as detected by salivary nitric oxide are collected pre-dose until the end of each treatment period and up to one week after cessation of treatment.

The PK sample and salivary nitric oxide collection time points are as follows:

Week 1: Pre-LA001 application prior to 0 hour and post-LA001 application at 2, 6, 12, 24, 36, 48, 60, 72, 84, 96, 108, 120, 132, 144, and 156 hours.

Week 2: 168 hours, and at up to 528 hours.

Skin irritation will be monitored throughout LA001 treatments. Safety will be monitored throughout the study by adverse event reporting, repeated clinical and laboratory evaluations.

### Inclusion Criteria:

### Healthy, Adult, Male or Female

Body mass index ≥18.0 and ≤32.0 kg/m2 at screening

Medically healthy with no clinically significant medical history, physical examination, laboratory profiles, vital signs or electrocardiograms (ECGs), as deemed by the Investigator.

Have a Fitzpatrick skin type of I, II or III or have skin colorimeter scores equivalent to the allowed Fitzpatrick skin type.

TABLE 15
Primary Outcome Measures

| Outcome Measure | Measure Description | Time Frame |
|---|---|---|
| Pharmacokinetics, | Area under the plasma concentration | Blood samples for donepezil PK will be |
| AUC | versus time curve (AUC) of once- | collected pre-dose until the end of each |
| | weekly LA001. | treatment period, approximately 18 weeks |
| | | total |
| Pharmacokinetics, | Peak Plasma Concentration (Cmax) of | Blood samples for donepezil PK will be |
| CMAX | once-weekly LA001. | collected pre-dose until the end of each |
| | | treatment period, approximately 18 weeks |
| | | total |
| Nitric Oxide | Salivary nitric oxide | |

TABLE 16
Secondary Outcome Measures

| Outcome Measure | Measure Description | Time Frame |
|---|---|---|
| Number of participants with | General Safety (adverse events and | Daily during 1 week |
| treatment-related adverse | serious adverse events as reported by | treatment period throughout |
| events as assessed by | subject following guidance CTCAE v4.0) | the 5 week period |
| CTCAE v4.0 | | |
| Summary Listing of Skin | Skin irritation score is determined by the | 0.5 hr, 24 hr, 48 hr and 72 |
| Irritation Score of LA001 by | sum of Dermal Response score (8-point | hr after end of treatment. |
| end of treatment course | categorical scale; where 0 = no evidence of | (3 days) |
| | irritation to 7 = strong reaction) using | |
| | numeric values) and the Other Effects | |
| | score (6-point categorical scale where | |
| | 0 = none observed to H = scabs/erosion | |
| | using alphabet letters equivalent to | |
| | numeric values) | |

### Example 16: A Molecular Application Platform (MAP) Emulsion

A Molecular Application Platform (MAP) emulsion was prepared using the following components: citric acid, ascorbic acid, citrulline malate, safflower oil, oleic acid, Tween 80, and water. The amounts of each component in preparing the emulsion were 4.8 g citric acid, 3.8 g ascorbic acid, 5.8 g citrulline malate, 26.1 g safflower oil, 2.6 g oleic acid, 13.0 g Tween 80, and 191 g water. Following the preparation, the emulsion was administered topically to a subject. The topical administration caused nitric oxide levels to increase in the subject.

### Example 17: Effect of Different Weight Percentages of a Non-Ionic Emulsifier (Polysorbate 80) on the Emulsion

A series of dermatological emulsions were prepared to evaluate the effect of a non-ionic emulsifier-polysorbate 80 on the physical stability of the emulsions.

Each emulsion was prepared by the same procedure as disclosed herein. Water was heated to a temperature of around 85° C. After heating the water was placed in a beaker with constant stirring, and the aqueous phase components—citric acid, citrulline malate (1:2), and ascorbic acid were added slowly ensuring constant mixing. After the complete mixing the aqueous phase mixture was allowed to cool to around 66° C. In a separate vessel, the contents of oil phase—safflower oil, oleic acid, and polysorbate 80 were mixed at room temperature (25° C.). The contents of the oil phase were mixed at around 800 rpm for 2-15 minutes at a temperature of about 50° C. After both the aqueous phase and the oil phase were prepared, the entire volume of the oil phase was added to the water phase while maintaining vigorous stirring throughout. The stirring continued at 800 RPM for 10-15 minutes at 50° C. to allow the emulsion to stabilize and mature.

Polysorbate 80 was incorporated at varying w % levels. The emulsion LAB2P104.4 comprises 20 g of polysorbate 80, and 10 g of citric acid, i.e., in a 2:1 w/w ratio. The emulsions LAB2P104.3, LAB2P104.2, and LAB2P104.1 comprise 15 g, 10 g, and 5 g, of polysorbate 80 respectively, whereas the amount of citric acid remained 10 g in all the emulsions. Therefore, the w/w ratio of polysorbate 80 to citric acid in the formulations LAB2P104.4, LAB2P104.3, LAB2P104.2, and LAB2P104.1 were 2:1, 1.5:1, 1:1, and 0.5:1, respectively.

Following table shows the components of the emulsions that were prepared according to the present example.

TABLE 17

| Components | LAB2P104.4 | | LAB2P104.3 | | LAB2P104.2 | | LAB2P104.1 | |
|---|---|---|---|---|---|---|---|---|
| Citric acid | 10 | g | 10 | g | 10 | g | 10 | g |
| Citrulline | 5.50 | g | 5.50 | g | 5.50 | g | 5.50 | g |
| malate (1:2) | | | | | | | | |
| Ascorbic acid | 4.40 | g | 4.40 | g | 4.40 | g | 4.40 | g |
| Water | 100 | g | 100 | g | 100 | g | 100 | g |
| Safflower oil | 8 | g | 8 | g | 8 | g | 8 | g |
| Oleic acid | 4 | g | 4 | g | 4 | g | 4 | g |
| Polysorbate 80 | 20 | g | 15 | g | 10 | g | 5 | g |

| | | | | |
|---|---|---|---|---|
| Ratio of | 2:1 | 1.5:1 | 1:1 | 0.5:1 |
| polysorbate | | | | |
| 80:citric acid | | | | |

Each emulsion was stored on ice, and the stability was assessed by visual inspection. The results are shown on the table below.

TABLE 18

| Polysorbate 80:citric | |
|---|---|
| acid w/w ratio | Visual Observation when stored on ice |
| 2:1 | No breakage observed at 24 hours |
| 1.5:1 | Around 25% breakage within 4 hours |
| 1:1 | Around 75% breakage within 4 hours |
| 0.5:1 | Around 100% breakage within 4 hours |

This data indicates that the non-ionic emulsifier, such as polysorbate 80 plays a role in maintaining the structural integrity of the emulsion. The emulsion that had 2:1 polysorbate 80 and citric acid did not display any breakage, whereas in the emulsions having lower ratios of polysorbate 80: citric acid, such as 1.5:1, 1:1, and 0.5:1 breakage up to different levels of the emulsions was observed within 4 hours when stored on ice. The emulsion with the ratio of polysorbate 80: citric acid of 0.5:1 exhibited 100% breakage within 4 hours when stored on ice.

### Example 18: Effect of the Inclusion of Different Weight Percentages of a Crosslinker on the Emulsion

A series of dermatological emulsions in the form of hydrogels were prepared to evaluate the effect of a crosslinker-hydroxypropyl methylcellulose (HPMC) on the physical stability of the emulsions in the form of hydrogels.

Each emulsion was prepared by the same procedure as disclosed herein. Water was heated to a temperature of around 85° C. After heating, the water was placed in a beaker with constant stirring, and the contents of aqueous phase—citric acid, citrulline malate (1:2), ascorbic acid, and HPMC (if required, as per the emulsion that was prepared) were added slowly ensuring constant mixing. After the complete mixing the combination of water, citric acid, and HPMC was allowed to cool to around 66° C. In a separate vessel, the contents of oil phase—safflower oil, oleic acid, and polysorbate 80 were mixed at room temperature (25° C.). The contents of the oil phase were mixed at around 800 rpm for 2-15 minutes at a temperature of about 50° C. After both the aqueous phase and the oil phase were prepared, the entire volume of the oil phase was added to the water phase while maintaining vigorous stirring throughout. The stirring continued at 800 RPM for 10-15 minutes at 50° C. to allow the emulsion to stabilize and mature.

The cross-linker—HPMC was incorporated at varying w % levels. The emulsion LAB2P096.1, LAB2P096.2, LAB2P096.3, and LAB2P096.4 comprises 1 g, 2 g, 4 g, and 0 g of HPMC, respectively, whereas the amount of citric acid remained 10 g in all the emulsions. Therefore, the w/w ratio of crosslinker (HPMC) to citric acid in the formulations LAB2P096.1, LAB2P096.2, LAB2P096.3 were 0.1:1, 0.2:1, and 0.4:1, respectively.

Following table shows the components of the emulsions that were prepared according to the present example.

TABLE 19

| Components | LAB2P096.1 | | LAB2P096.2 | | LAB2P096.3 | | LAB2P096.4 | |
|---|---|---|---|---|---|---|---|---|
| Citric acid | 10 | g | 10 | g | 10 | g | 10 | g |
| Citrulline | 6 | g | 6 | g | 6 | g | 6 | g |
| malate (1:2) | | | | | | | | |
| Ascorbic acid | 4 | g | 4 | g | 4 | g | 4 | g |
| Water | 100 | g | 100 | g | 100 | g | 100 | g |
| Safflower oil | 8.7 | g | 8.7 | g | 8.7 | g | 8.7 | g |
| Oleic acid | 3.92 | g | 3.92 | g | 3.92 | g | 3.92 | g |
| Polysorbate 80 | 17.40 | g | 17.40 | g | 17.40 | g | 17.40 | g |
| HPMC | 1 | g | 2 | g | 4 | g | 10 | g |

| | | | | |
|---|---|---|---|---|
| Ratio of | 0.1:1 | 0.2:1 | 0.4:1 | — |
| HPMC:citric | | | | |
| acid | | | | |

Each emulsion was stored at room temperature, and the stability was assessed by visual inspection. The results are shown on the table below.

TABLE 20

| HPMC:citric | Visual Observation when stored |
|---|---|
| acid w/w ratio | at room temperature |
| (No HPMC) | No breakage observed at 4 hours |
| 0.1:1 | Around 25% breakage within 4 hours |
| 0.2:1 | Around 75% breakage within 4 hours |
| 0.4:1 | Around 100% breakage within 4 hours |

This data indicates a correlation between the concentration of HPMC and the rate of hydrogel breakage. At 0% HPMC, the hydrogels exhibited stability with no observed breakage. However, when HPMC was added, the hydrogel integrity decreased, leading to higher rates of breakage. And in the emulsion where HPMC was added at a 40% weight vs citric acid (0.4:1 w/w ratio), complete (100%) breakage within the 4-hour period was observed.

### Example 19: Preparation of a Transdermal Composition: LAB2P1

### Preparation of Combination A

All materials for Combination A were assembled at room temperature (25° C.). In a 2000 ml dry mixing beaker, the following powdered ingredients were carefully weighed and combined in the order listed: citric acid 300 g (1.56 mol), citrulline 1:2 malate 165 g (0.53 mol), ascorbic acid 132 g (0.75 mol), EEA 60 g (0.43 mol), magnesium citrate 15 g (0.01 mol), calcium-HMB 7.5 g (0.03 mol), potassium citrate 7.5 g (0.02 mol), ferrous gluconate 0.9 g, creatine 30 g, leucine 30 g (0.23 mol), and xanthan gum 30 g. Once all components had been added to the vessel, the dry blend was mixed at a low speed of 10 RPM for 5 minutes to ensure uniform distribution of all ingredients. This combination was then set aside for later use.

### Water Preparation and Initial Mixing

While Combination A was resting, the water phase was prepared by heating 1800 g of water to 85° C. in an external heating container. When the temperature had been reached, the heated water was added to a 5000 ml beaker equipped with an overhead stirrer. Once the water had been added, stirring began at 800 RPM for 1 minute to establish a stable vortex and ensure uniform temperature throughout the liquid.

### Incorporation of Combination A

The prepared Combination A was gradually added to the heated water with stirring over the course of 1 minute. The powder was added slowly and steadily to prevent clumping and to ensure proper hydration of all components, particularly the xanthan gum and other hydrophilic ingredients. After the complete addition of Combination A, stirring continued at 800 RPM for 5 minutes while the mixture cooled from 85° C. down to 60° C. During this time, the mixture was observed for complete dissolution, uniform consistency, and an increase in viscosity (approximately 10-14 kPa).

### Preparation of Combination B

While the main mixture was stirring and cooling, Combination B was prepared in a separate 20000 ml beaker. At room temperature (25° C.), the following components of oil phase were combined: polysorbate 80 (PS80) 1050 g (1.74 mol), safflower oil 375 g (1.34 mol), oleic acid 199.8 g (0.71 mol), tea tree oil 9 g (0.04 mol), and jojoba oil 9 g (0.01 mol). Once all the oil phase components had been combined, the oil phase was heated to 50° C. while mixing at 800 RPM for 2 minutes to ensure complete homogenization and uniform temperature distribution throughout the oil blend.

### Integration of Oil Phase

After both the aqueous phase (containing Combination A) and the oil phase (Combination B) had been adequately prepared, the entire volume of Combination B was slowly added to the main mixture over 1 minute, maintaining vigorous stirring throughout to form a stable emulsion. Once Combination B had been completely incorporated, mixing continued at 800 RPM for 10 minutes at 50° C. to allow the emulsion to stabilize and mature.

### Final Component Addition

After the emulsion had been thoroughly mixed, the temperature was reduced to 25° C. and 45 g (0.29 mol) of menthol was carefully added to the mixture. Menthol was added at room temperature to prevent excessive volatilization. Once menthol had been added, the final formulation was mixed at the previous stirring rate for 5 minutes, maintaining a temperature of 35° C. to ensure even distribution of the menthol throughout the product.

The final product had a total batch weight of approximately 4265.7 g. Once the final mixing step had been completed, the preparation was ready for transfer to appropriate containers or for further processing as required by the specific application.

TABLE 21

| | Amount | Ratio Wt/Wt | | Weight/ |
|---|---|---|---|---|
| Components | Calc (g) | (w.r.t citric acid) | M | weight % |
| Citric Acid | 300.00 | 1.000 | 0.366 | 7.03% |
| Citrulline 1:2 | 165.00 | 0.550 | 0.125 | 3.87% |
| Malate | | | | |
| Ascorbic Acid | 132.00 | 0.440 | 0.176 | 3.09% |
| EEA | 60.00 | 0.200 | 0.102 | 1.41% |
| Magnesium | 15.00 | 0.0500 | 0.002 | 0.35% |
| Citrate | | | | |
| ca-HMB | 7.50 | 0.025 | 0.006 | 0.18% |
| Potassium Citrate | 7.50 | 0.025 | 0.006 | 0.18% |
| Ferrous Gluconate | 0.90 | 0.003 | 0.000 | 0.02% |
| Creatine | 30.00 | 0.100 | 0.000 | 0.70% |
| Leucine | 30.00 | 0.100 | 0.054 | 0.70% |
| Xanthan gum | 30.00 | 0.100 | 0.000 | 0.70% |
| Water | 1,800.00 | 6.000 | 23.423 | 42.20% |
| PS80 | 1,050.00 | 3.500 | 0.407 | 24.61% |
| Safflower Oil | 375.00 | 1.250 | 0.313 | 8.79% |
| Oleic Acid | 199.80 | 0.666 | 0.166 | 4.68% |
| Tea Tree | 9.00 | 0.030 | 0.010 | 0.21% |
| Jojoba Oil | 9.00 | 0.030 | 0.003 | 0.21% |
| Menthol | 45.00 | 0.150 | 0.068 | 1.05% |

### Example 20: Preparation of a Transdermal Composition: LAB32P2

### Preparation of Combination A

In a 2000 ml dry mixing beaker, 300 g of citric acid was added. Further, Primary active ingredients were added: 1. Citrulline 1:2 Malate—165 g; 2. Ascorbic Acid—132 g; 3. EEA (Essential Amino Acids)—60 g; 4. Collagen—60 g; 5. Creatine—60 g; 6. Biotin—60 g; 7. Theanine—60 g; 8. Glycine—90 g; 9. GABA—60 g; and 10. Taurine—60 g. Further, secondary active ingredients were added to the above mixture: 11. Armica Extract—45 g (2 g material+43 g water); 12. Saw Palmetto—45 g; 13. Vitamin E—45 g; 14. B12 Complex—22.5 g; 15. D-Ribose—22.5 g; 16. Beta Carotene—10.5 g; 17. Folic Acid—10.5 g; 18. Hyaluronic Acid—5.25 g; and 19. Vitamin D—5.25 g. In the next step, the following mineral ingredients were added: 20. Manganese Gluconate—3 g; 21. Zinc Gluconate—3 g; 22. Copper Gluconate—0.6 g; 23. Chromium GTF—0.06 g; 24. Magnesium Citrate—15 g (2 g material+13 g water); 25. Calcium HMB—7.5 g; 26. Potassium Citrate—7.5 g; and 27. Ferrous Gluconate—0.9 g. In the next step, additional active ingredients were added: 28. Creatine (additional)—30 g; 29. Leucine—30 g; and 30. Xanthan Gum (thickener)—30 g. Once all components had been added to the vessel, the dry blend was mixed at room temperature at a low speed of 10 RPM for 5 minutes to ensure uniform distribution of all ingredients. This combination was then set aside for later use.

### Water Preparation and Initial Mixing

While Combination A was resting, the water phase was prepared by heating 1800 g of water to 85° C. in an external heating container. When the temperature had been reached, the heated water was added to a 5000 ml beaker equipped with an overhead stirrer. Once the water had been added, stirring began at 800 RPM for 1 minute to establish a stable vortex and ensure uniform temperature throughout the liquid.

### Incorporation of Combination A

The prepared Combination A was gradually added to the heated water with stirring over the course of 1 minute. The powder was added slowly and steadily to prevent clumping and to ensure proper hydration of all components, particularly the xanthan gum and other hydrophilic ingredients. After the complete addition of Combination A, stirring continued at 800 RPM for 5 minutes while the mixture cooled from 85° C. down to 60° C. During this time, the mixture was observed for complete dissolution, and uniform consistency.

### Preparation of Combination B

While the main mixture was stirring and cooling, Combination B was prepared in a separate 20000 ml beaker. At room temperature (25° C.), the following components of oil phase were combined: polysorbate 80 (PS80) 1050 g (1.74 mol), safflower oil 375 g (1.34 mol), oleic acid 199.8 g (0.71 mol), tea tree oil 9 g (0.04 mol), and jojoba oil 9 g (0.01 mol). Once all the oil phase components had been combined, the oil phase was heated to 50° C. while mixing at 800 RPM for 2 minutes to ensure complete homogenization and uniform temperature distribution throughout the oil blend.

### Integration of Oil Phase

After both the aqueous phase (containing Combination A) and the oil phase (Combination B) had been adequately prepared, the entire volume of Combination B was slowly added to the main mixture over 1 minute, maintaining vigorous stirring throughout to form a stable emulsion. Once Combination B had been completely incorporated, mixing continued at 800 RPM for 10 minutes at 50° C. to allow the emulsion to stabilize and mature.

### Final Component Addition

After the emulsion had been thoroughly mixed, the temperature was reduced to 25° C. and 45 g (0.29 mol) of menthol was carefully added to the mixture. Menthol was added at room temperature to prevent excessive volatilization. Once menthol had been added, the final formulation was mixed at the previous stirring rate for 5 minutes, maintaining a temperature of 35° C. to ensure even distribution of the menthol throughout the product.

The final product had a total batch weight of approximately 4933.86 g. Once the final mixing step had been completed, the preparation was ready for transfer to appropriate containers or for further processing as required by the specific application.

TABLE 22

| | Amount | Ratio Wt/Wt | | Weight/ |
|---|---|---|---|---|
| Components | Calc (g) | (w.r.t citric acid) | M | weight % |
| Citric Acid | 300.00 | 1.000 | 0.366 | 7.03% |
| Citrulline 1:2 | 165.00 | 0.550 | 0.125 | 3.87% |
| Malate | | | | |
| Ascorbic Acid | 132.00 | 0.440 | 0.176 | 3.09% |
| Essential Amino | 60.00 | 0.200 | 0.102 | 1.41% |
| Acids (EEA) | | | | |
| Collagen | 60.00 | 0.200 | 0.010 | 1.22% |
| Creatine | 60.00 | 0.200 | 0.000 | 1.22% |
| Biotin | 60.00 | 0.200 | 0.246 | 1.22% |
| Theanine | 60.00 | 0.200 | 0.344 | 1.22% |
| Glycine | 90.00 | 0.300 | 1.199 | 1.82% |
| Gaba | 60.00 | 0.200 | 0.582 | 1.22% |
| Taurine | 60.00 | 0.200 | 0.479 | 1.22% |
| arnica | 45.00 | 0.1500 | 0.010 | 0.91% |
| Saw Pametto | 45.00 | 0.1500 | 0.156 | 0.91% |
| Vitamin E | 45.00 | 0.1500 | 0.104 | 0.91% |
| B12 Complex | 22.50 | 0.0750 | 0.017 | 0.46% |
| D-Ribose | 22.50 | 0.0750 | 0.000 | 0.46% |
| Beta Carotene | 10.50 | 0.0350 | 0.020 | 0.21% |
| Folic Acid | 10.50 | 0.0350 | 0.024 | 0.21% |
| Hyaluronic acid | 5.25 | 0.0175 | 0.010 | 0.11% |
| Vitamin D | 5.25 | 0.0175 | 0.014 | 0.11% |
| MnGluconate | 3.00 | 0.010 | 0.000 | 0.06% |
| Zn Gluconate | 3.00 | 0.010 | 0.007 | 0.06% |
| Cu Gluconate | 0.60 | 0.002 | 0.000 | 0.01% |
| Chromium GTF | 0.06 | 0.000 | 0.000 | 0.00% |
| Magnesium | 15.00 | 0.0500 | 0.002 | 0.35% |
| Citrate | | | | |
| ca-HMB | 7.50 | 0.025 | 0.006 | 0.18% |
| Potassium Citrate | 7.50 | 0.025 | 0.006 | 0.18% |
| Ferrous Gluconate | 0.90 | 0.003 | 0.000 | 0.02% |
| Creatine | 30.00 | 0.100 | 0.000 | 0.70% |
| Leucine | 30.00 | 0.100 | 0.054 | 0.70% |
| Xanthan gum | 30.00 | 0.100 | 0.000 | 0.70% |
| Water | 1,800.00 | 6.000 | 23.423 | 42.20% |
| PS80 | 1,050.00 | 3.500 | 0.407 | 24.61% |
| Safflower Oil | 375.00 | 1.250 | 0.313 | 8.79% |
| Oleic Acid | 199.80 | 0.666 | 0.166 | 4.68% |
| Tea Tree | 9.00 | 0.030 | 0.010 | 0.21% |
| Jojoba Oil | 9.00 | 0.030 | 0.003 | 0.21% |
| Menthol | 45.00 | 0.150 | 0.068 | 1.05% |

### Example 21: Long-Term Blood Pressure-Lowering Effects of Systemic NO-Increasing Emulsions

This example demonstrates the long term decreases in blood pressure, increases in NO, and increase in Sp02 that result after daily administration of a systemic NO-increasing emulsion provided herein.

### Materials and Methods

A series of systemic NO-increasing emulsions were prepared over a period of around 4 years. Components of these NO-promoting emulsions included NO-increasing agents (either beetroot, arginine, and/or citrulline as nitric oxide-promoting agents), linoleic acid, or a source thereof, oleic acid, or a source thereof, citric acid, and polysorbate 80 as the emulsifier. The emulsions had pH between 3.2 and 4.5 during approximately years 1 and 2, and pH between 4.5 and 5.5 for approximately the last 2 years.

More specifically, in addition to the above characteristics and components, the systemic nitric oxide-increasing emulsions that were administered for the last year of the trial met these characteristics:
citric acid, optionally with a source of acetic acid (weak organic acid(s)), combined in total in an amount from 0.2% to 45.0% w/w of the emulsion;
the total systemic-nitric oxide-increasing agents were present in a weight ratio from 0.1:1.0 to 3.0:1.0 relative to the weak organic acid(s) in total;
water, present in a weight ratio from 0.5:1 to 45:1 relative to the weak organic acid(s) in total;
the linoleic acid and/or oleic acid, or a source thereof, were present in a weight ratio from 0.2:1.0 to 4.0:1.0 relative to the weak organic acid(s) in total; and
polysorbate 80 in total present in a weight ratio from 1.0:1.0 to 5.0:1.0 relative to the weak organic acid(s) in total.

Furthermore, the total fatty acids, and/or C10 or longer non-fatty acid hydrocarbons if present in the emulsion, comprised less than or equal to a total of 15.0% w/w of saturated fatty acids and/or saturated C10 or longer non-fatty acid hydrocarbons,

LAB1_TDP (see Table LAB1_TDP) is a representative NO-increasing emulsion that was one of the systemic nitric acid-increasing emulsions prepared and administered by the human subject during the 2nd 2 years of the study. It is noteworthy that LAB1_TDP includes the direct NO precursors citrulline malate and arginine. Furthermore LAB1_TDP includes beetroot as a plant nitrate source as well as the NO-supporting agent, creatine. Also note that the gram weights are provided for relative comparison purposes only and not the batch size actually prepared.

The LAB1_TDP emulsion was made as follows: The aqueous phase liquid components were combined and mixed at 38 C for 20 minutes at 500 RPM. The dry components of the LAB1_TDP formulation were combined and mixed for 5 minutes at 25 C. Then the mixed dry components were added to the 38 C mixed aqueous components while mixing at 1,000 RPM and incubated at 38 C for an additional 5 minutes while mixing at 1,000 RPM to form the complete aqueous phase.

The organic phase components were combined together and mixed at 25 C for 5 minutes to form the mixed organic phase liquid. Then the mixed organic phase liquid was added to the complete aqueous phase liquid at 38 C while mixing at 1,500 RPM to form the complete LAB1_TDP formulation. Then the complete LAB1_TDP formulation was mixed at 38 C for 30 minutes at 1,500 RPM to form the LAB1_TDP emulsion.

The subject topically administered 0.5 to 1.5 ounces of the systemic NO-increasing emulsions as discussed above daily for a period of around 4 years.

### Results

Upon daily topical administration of 0.25 to 1.5 ounces of the systemic NO-increasing emulsions, the subject experienced a long-term drop in blood pressure from around 130/70 to around 100/65 mmHg that remained at/near the lower levels for the final 4 years of the trial. Furthermore, the subject's SpO2 increased from around 900 to around 950 w readings were taking a year after the initial administration, and then remained at the higher level for the remaining 3 years of the trial. Furthermore, heart rate dropped from about 85 bpm to about 70 bpm. Finally, elevated levels of NO were observed for the final year of the trial, which was the entire period they were measured for this trial.

Thus, daily administration of emulsions herein, were demonstrated to be systemic NO-increasing emulsions, and such increased systemic NO levels were sustained for the entire year it was measured. Furthermore, the emulsions caused a multi-year decrease in blood pressure and heart rate, and a multi-year increase in SpO2.

TABLE
LAB1P_TDP: Components of LAB1P_TDP emulsion

| | Ratio | Ratio | | Amount | |
|---|---|---|---|---|---|
| Component | Wt/Wt | Mol/Mol | Mol | Calc (g) | % (wt) |
| Aqueous phase | | | | | |
| liquid | | | | | |
| Citric Acid | 1.00 | 1.00 | 0.21 | 40.00 | 17.5% |
| Water* | 1.50 | 16.00 | 3.33 | 60.00 | 26.3% |
| Acetic Acid | 0.75 | 2.40 | 0.50 | 30.00 | 13.2% |
| Powder | — | — | — | — | |
| Citrulline malate | 0.10 | 0.06 | 0.01 | 4.00 | 1.8% |
| Arginine | 0.05 | 0.06 | 0.01 | 2.00 | 0.9% |
| Beetroot | 0.10 | 0.03 | 0.01 | 4.00 | 1.8% |
| CREATINE | 0.05 | 0.07 | 0.02 | 2.00 | 0.9% |
| SERINE | 0.05 | 0.09 | 0.02 | 2.00 | 0.9% |
| Kale | 0.05 | 0.05 | 0.01 | 2.00 | 0.9% |
| EEA | 0.05 | 0.02 | 0.01 | 2.00 | 0.9% |
| Lecithin (SunF) | 0.05 | 0.01 | 0.00 | 2.00 | 0.9% |
| Oil Phase | | | | | |
| Macadamia Nut Oil | 0.10 | | | 4.00 | 1.8% |
| Maracuja Oil | 0.25 | | | 10.00 | 4.4% |
| Glycerin | 0.10 | 0.21 | 0.04 | 4.00 | 1.8% |
| PS80 | 1.50 | 0.48 | 0.10 | 60.00 | 26.3% |

*Note that PS80 contains 4.00 g of water. Thus, water content is 28.1% (26.3% + 1.8%)

One of ordinary skill in the art will appreciate that starting materials, biological materials, reagents, synthetic methods, purification methods, analytical methods, assay methods, and biological methods other than those specifically exemplified can be employed in the practice of the invention without undue experimentation. All art-known functional equivalents of any such materials and methods are intended to be included in this invention.

The foregoing description and accompanying drawings set forth a number of representative embodiments at the present time. Various modifications, additions, and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A transdermal delivery formulation, comprising:
at least one weak organic acid present in total in an amount from 2.0% to 45.0% w/w of the formulation;
at least one active agent;
water, present in a weight percentage in the range of 15% to 70% of the formulation;
one or more oils comprising at least one unsaturated long-chain fatty acid selected from oleic acid and linoleic acid, wherein the one or more oils in total have a weight percentage in the range of 5% to 35% of the formulation; and
polysorbate 80 present in a weight percentage in the range of 15% to 40% of the formulation,
wherein the transdermal delivery formulation has a pH in the range of 2.0 to 6.0,
wherein the polysorbate 80, the one or more oils in total, and the water comprises a weight ratio in the range of X:Y:Z, wherein X is =1-5, Y is =1-6, and Z is =1-6, and
wherein the transdermal delivery formulation is capable of delivering the at least one agent to the blood stream of a subject when applied to the skin of the subject.

2. The transdermal delivery formulation of claim 1, wherein the at least one active agent comprises arginine, citrulline, citrulline malate, beetroot, creatine, glutamine, leucine, norvaline, ornithine, histidine, beta-alanine, phenylalanine, agmatine, betaine, L-theanine, glutathione, or any nitrate or any nitrite, any nitro, or any nitroso or salt thereof, or any combination thereof.

3. The transdermal delivery formulation of claim 1, wherein the at least one active agent further comprises ascorbic acid or its derivative.

4. The transdermal delivery formulation of claim 1, wherein at least one weak organic acid is present in total in an amount from 4.5% to 45.0% w/w.

5. The transdermal delivery formulation of claim 1, wherein the at least one weak organic acid is present in total in an amount from 10.0% to 45.0% w/w.

6. The transdermal delivery formulation of any one of claim, wherein the transdermal delivery formulation comprises about 20%, about 25%, about 30%, about 35%, about 40%, about 45%, about 50%, about 55%, about 60%, about 65%, about 70%, about 75%, or about 80% (w/w) of the at least one weak organic acid.

7. The transdermal delivery formulation of any one of claim, wherein the transdermal delivery formulation comprises about 60%, about 65%, about 70%, about 75%, or about 80% w/w of the total of the polysorbate 80, the one or more oils in total, and water.

8. The transdermal delivery formulation of any one of claim, wherein the at least one weak organic acid comprises 2 weak organic acids at a ratio by weight of about 1:1, 1:2, 1:3, 1:4, 1:5, 1:6, 1:7, 1::8, 1:9, 1:10, 1:100 or 1:1000 relative to each other.

9. The transdermal delivery formulation of any one of claim, wherein the at least one weak organic acid comprises about 80%, about 85%, about 90%, about 95%, about 97%, about 99% (w/w) of one or more weak organic acids.

10. The transdermal delivery formulation of any one of claim, wherein the at least one weak organic acid has a median pKa of from 3.0 to 5.5.

11. The transdermal delivery formulation of any one of claim, wherein the at least one weak organic acid has a median pKa of from 4.0 to 5.0.

12. The transdermal delivery formulation of any one of claim, wherein the at least one weak organic acid has a median pKa of about 4.6.

13. The transdermal delivery formulation of any one of claim, wherein the at least one weak organic acid comprises a mono, di or tri carbonic acid of chain length (R) between 1-16.

14. The transdermal delivery formulation of any one of claim, wherein the at least one weak organic acid comprises a mono or poly hydroxy moiety of 0-14.

15. The transdermal delivery formulation of any one of claim, wherein the at least one weak organic acid comprises lactic acid, acetic acid, formic acid, citric acid, oxalic acid, gallic acid, malic acid, maleic acid, malonic acid, succinic acid, tartaric acid, fumaric acid, or any combination thereof.

16. The transdermal delivery formulation of any one of claim, wherein said weak organic acid is citric acid or acetic acid.

17. The transdermal delivery formulation of any one of claim, wherein the at least one weak organic acid comprises citric acid and acetic acid.

18. The transdermal delivery formulation of claim 17, wherein the acetic acid is apple cider vinegar.

19. The transdermal delivery formulation of any one of claim, wherein the formulation comprises two oils comprising an unsaturated long-chain fatty acid, wherein the two oils are in a ratio by weight of about 1:1, 1:2, 1:3, 1:4, 1:5, 1:6, 1:7, 1:8, 1:9, 1:10, 1:100 or 1:1000 relative to each other.

20. The transdermal delivery formulation of any one of claim, wherein said at least one unsaturated long-chain fatty acid comprises a chain of from 12 to 26 carbons.

21. The transdermal delivery formulation of claim 20, wherein said at least one unsaturated long-chain fatty acid comprises one or more double bonds in a cis configuration.

22. The transdermal delivery formulation of claim 20, wherein said at least one unsaturated long-chain fatty acid comprises adrenic acid, arachidonic acid, arachidic acid, behenic acid, brassidic acid, cervonic acid, cis-vaccenic acid, dihomo-γ-linolenic acid, docosadienoic acid, eicosadienoic acid, eicosapentaenoic acid, eicosatetraenoic acid, eicosenoic acid, elaidic acid, erucic acid, gadoleic acid, gondoic acid, herring acid, lauric acid, lignoceric acid, linoleic acid, linolelaidic acid, margaric acid, margoleic acid, mead acid, myristoleic acid, nervonic acid, oleic acid, ozubondo acid, palmitic acid, palmitoleic acid, trans-palmitoleic acid, paullinic acid, petroselinic acid, pinolenic acid, sapienic acid, sardine acid, stearic acid, stearidonic acid, tetracosapentaenoic acid, α-linolenic acid, γ-linolenic acid, or any combination thereof.

23. The transdermal delivery formulation of claim 22, wherein said at least one unsaturated long-chain fatty acid is oleic acid or linoleic acid.

24. The transdermal delivery formulation of claim 22, wherein said at least one long-chain fatty acid comprises oleic acid and linoleic acid.

25. The transdermal delivery formulation of any one of claim, wherein the one or more oils comprising an unsaturated long-chain fatty acid is one or more plant or animal oils.

26. The transdermal delivery formulation of claim 25, wherein said one or more plant oils is selected from the group consisting of vegetable oil, nut oil and seed oil.

27. The transdermal delivery formulation of claim 25, wherein said one or more plant or animal oils comprises macadamia oil, maracuja (passion fruit) oil, safflower oil, sunflower oil, olive oil, avocado oil, canola oil, coconut oil, corn oil, cottonseed oil, flaxseed/linseed oil, grape seed oil, hemp seed oil, palm oil, peanut oil, rice bran oil, sesame oil, soybean oil, brazil nut oil, almond oil, walnut oil, pecan oil, jojoba oil, chia seed oil, wallflower seed, mustard oil, borage oil, black currant oil, evening primrose oil, chicken fat, cartilage oil, cod liver oil, herring oil, mackerel oil, salmon oil, menhaden oil, sardine oil, or any combination thereof.

28. The transdermal delivery formulation of claim 27, wherein said one or more plant or animal oils is macadamia oil or maracuja (passion fruit) oil, or a combination thereof.

29. The transdermal delivery formulation of claim 27, wherein said one or more plant or animal oils comprises macadamia oil and maracuja (passion fruit) oil.

30. The transdermal delivery formulation of any one of claim, wherein the transdermal delivery formulation comprises a lotion, a gel, a cream, an ointment, a liniment, a paste, a film, an encapsulation, or a liquid.

31. The transdermal delivery formulation of any one of claim, wherein the formulation comprises a nitrate source.

32. The transdermal delivery formulation of claim 31, wherein said nitrate source is a plant-based nitrate source.

33. The transdermal delivery formulation of claim 32, wherein said plant-based nitrate source comprises from the group consisting of arugula, spinach, leafy green vegetables, beetroot, or any combination thereof.

34. The transdermal delivery formulation of claim 33, wherein said plant-based nitrate source is beetroot, and wherein the beetroot is a dried beetroot powder.

35. The transdermal delivery formulation of claim 34, wherein the formulation comprises about 2.0%, about 2.5%, about 3.0%, about 3.5%, about 4.0%, about 4.5%, about 5.0%, about 5.5%, about 6.0%, about 6.5%, about 7.0%, about 7.5%, or about 8.0% w/w dried beetroot powder.

36. The transdermal delivery formulation of any one of claims, further comprising a viscosity enhancer, a nutrient, a plant powder or extract, an amino acid, a vitamin, or any combination thereof.

37. The transdermal delivery formulation of claim 36, wherein said formulation comprises a viscosity enhancer selected from the group consisting of lecithin, aloe vera, glycerin, a plant oil, an animal oil, and collagen.

38. The transdermal delivery formulation of claim 36, wherein said formulation comprises a nutrient selected from the group consisting of acetyl-L-carnitine, alpha lipoic acid, arginine, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

39. The transdermal delivery formulation of claim 36, wherein said formulation comprises a plant powder or extract selected from the group consisting of bacopa powder, bamboo extract powder, beet powder, blueberry extract, ginkgo biloba, ginger, grape seed extract, green tea, jojoba, nutmeg, olive leaf, pomegranate, and turmeric.

40. The transdermal delivery formulation of claim 36, wherein said formulation comprises an amino acid selected from the group consisting of alanine, arginine, leucine, isoleucine, valine, glutamine, glycine, histidine, leucine, lysine, methionine, proline, serine, threonine, and valine.

41. The transdermal delivery formulation of claim 36, wherein said formulation comprises a vitamin selected from the group consisting of vitamin A, vitamin B-1, vitamin B-2, vitamin B-3, vitamin B-7 & 8 inositol, vitamin B-9 (folic acid), vitamin B-12, vitamin C, vitamin D-3, and vitamin E.

42. The transdermal delivery formulation of any one of claim, wherein said transdermal formulation has a pH of from 3.0 to 5.0.

43. The transdermal delivery formulation of any one of claim, wherein said transdermal formulation has a pH of from 3.5 to 4.5.

44. The transdermal delivery formulation of any one of claim, wherein said transdermal formulation has a viscosity at 20° C. of from 500 to 10,000 centipoise (cP) or from 1,000 to 5,000 cP, or from 1,500 to 4,000 cP, or from 2,000 to 3,000 cP.

45. The transdermal delivery formulation of any one of claim, wherein the transdermal delivery formulation forms an emulsion comprising a population of droplets from about 10 nm to about 300 nm in diameter.

46. The transdermal delivery formulation of any one of claims, further comprising one or more components for enhancing nutrition and optimizing health, immune support, restful sleep, exercise performance and nitric oxide metabolism, cognitive function, or any combination thereof.

47. The transdermal delivery formulation of claim 46, further comprising one or more of a hydration electrolyte, cinnamic acid, phenylalanine, resveratrol, carnitine.

48. The transdermal delivery formulation of claim 46, further comprising one or more of gamma-aminobutyric acid (GABA), melatonin, and tryptophan.

49. The transdermal delivery formulation of claim 46, further comprising one or more of vitamin D, vitamin C, citrulline, ribose ATP blood carrier, beetroot, curcumin (turmeric), fish oil, and threonine.

50. The transdermal delivery formulation of claim 46, further comprising one or more of silicon, leucine, lysine, isoleucine, valine, threonine, phenylalanine, methionine, histidine, tryptophan, vitamin B-5 (pantothenic acid), vitamin B-7 (biotin), vitamin K, acetyl L carnitine, palmitoyl-ethanolamide (PEA), taurine, periwinkle, artichoke, bacopa, ginkgo biloba, nutmeg, alanine, and tyrosine.

51. The transdermal delivery formulation of claim 1, wherein the formulation comprises:
about 12.0% (w/w) citric acid powder;
about 20.5% (w/w) apple cider vinegar;
about 1.4% (w/w) beetroot powder;
about 23.8% (w/w) polysorbate 80;
about 22.5% (w/w) distilled water;
about 9.9% (w/w) macadamia oil; and
about 9.8% (w/w) maracuja oil.

52. The transdermal delivery formulation of claim 1, wherein the formulation comprises:
about 374 g citric acid powder;
about 640 g apple cider vinegar;
about 44.4 g beetroot powder;
about 742 g polysorbate 80;
about 700 g distilled water;
about 308 g macadamia oil; and
about 308 g maracuja oil.

53. The transdermal delivery formulation of any one of claims, further comprising one or more additional ingredients, wherein the additional ingredients comprise: acetyl-l-carnitine (alc), alanine, alpha lipoic acid, arginine, artichoke extract, ashwagandha, bacopa powder, bamboo extract powder, basil powder, beet powder, calcium carbonate, blueberry extract, aloe vera, choline bitartrate, collagen protein peptides, collagen powder, citrulline malate, curcumin powder, creatine, CoQ10, kale powder, folic acid, gingko biloba, glutamine, glycerin, ginger, glycine, grape seed extract, green tea, inositol (vitamins B7/B8), histidine, jojoba, pomegranate powder, magnesium glycinate, leucine, lysine, lecithin (sun flower), niacinamide (vitamin B3), nutmeg powder, niacin, methionine, NALT (acetyl tyrosine), NAC, lutein, olive leaf, pea powder, periwinkle, phenylalanine, potassium, proline, ribose, DAA (d-aspartic acid), serine, hyaluronic acid, taurine, threonine, tryptophan, turmeric, theanine, valine (amino acid), valerian root powder, zinc oxide, vitamin A, vitamin B complex, vitamin B-1, vitamin B-2, vitamin B-5, vitamin B-6 (pyridoxine), vitamin B-7&8, vitamin B-9 (folic acid), vitamin B-12, vitamin C (ascorbic acid), vitamin D-3 (cholecalciferol), vitamin E, vitamin K-2, ginseng, isoleucine, almond oil, broccoli seed oil, collagen liquid, avocado oil, chamomile liquid, vitamin E oil, glycerin (conditioning), grapefruit seed, grape seed oil, gotu kola oil, kava liquid, virgin algae oil (fish oil), jojoba extract oil (organic), lavender oil, macadamia nut oil, meadowfoam seed oil, peppermint oil, maracuja oil (passionfruit), primrose oil, pomegranate oil, BCAA, GABA, collagen oil, virgin algae oil, ashwagandha powder, collagen peptides, guarana powder, and huperzine.

54. The transdermal delivery formulation of claim 53, wherein the transdermal delivery formulation comprises more than one, more than 2, more than 3, more than 4, more than 5, more than 6, more than 7, more than 8, more than 9, more than 10, more than 11, more than 12, more than 13, more than 14, more than 15, more than 16, more than 17, more than 18, more than 19, more than 20 of the additional ingredients.

55. The transdermal delivery formulation of any one of claim, wherein the one or more active agents comprises a therapeutic agent, a nutraceutical, or a combination thereof.

56. The transdermal delivery formulation of claim 55, wherein the therapeutic agent comprises a biologic, a protein, a peptide, a small molecule, a macromolecule, a nucleic acid, another pharmaceutically or physiologically active ingredient, or any combination thereof.

57. A method of transdermal delivery to a subject, comprising epicutaneous application of the transdermal delivery formulation of any one of claims 1-56 to the subject.

58. The method of claim 57, further comprising overlaying an ionized water on to the epicutaneously-applied transdermal delivery formulation.

59. A method of treatment of a disease or condition in a subject, comprising epicutaneous application of the transdermal delivery formulation of any one of claims 1-56 and one or more active agents to the subject.

60. The method of claim 59, wherein the one or more active agents comprises a therapeutic agent, a nutraceutical, or a combination thereof.

61. The method of claim 59, wherein the therapeutic agent comprises a biologic, a protein, a peptide, a small molecule, a macromolecule, a nucleic acid, another pharmaceutically or physiologically active ingredient, or any combination thereof.

62. A method of enhancing a feature or condition in a subject, comprising epicutaneous application of the transdermal delivery formulation of any one of claims 1-56 to the subject.

63. The method of claim 62, wherein the feature is a cosmetic feature.

64. The method of claim 62, wherein the condition is nutrition, general health, quality of sleep, exercise performance, nitric oxide metabolism, cognitive function, or any combination thereof.

65. A method of systemic delivery of an active agent to a subject, comprising epicutaneous application of the transdermal delivery formulation of any one of claims 1-56 and one or more active agents to the subject.

66. The method of systemic delivery of claim 65, further comprising overlaying an ionized water on to the epicutaneously-applied transdermal delivery formulation.

67. The method of claim 65, wherein the one or more active agents comprises a therapeutic agent, a nutraceutical, or a combination thereof.

68. The method of claim 65, wherein the therapeutic agent comprises a biologic, a protein, a peptide, a small molecule, a macromolecule, a nucleic acid, another pharmaceutically or physiologically active ingredient, or any combination thereof.
